(19) United States
(12) Patent Application Publication
(10) Pub. No.: US 2005/0050591 A1
(43) Pub. Date: Mar. 3, 2005

(54) Novel plant cyclin

(76) Inventors: Pal Miskolczi, Keoskernet (HU); Aladar Pettko-Szandtner, Szeged (HU); Gabor Horvath, Szeged (HU); Denes Dudits, Szeged (HU); Attila Feher, Szeged (HU); Janos Gyobgyey, Deszk (HU)

Correspondence Address: Ann R Pokalsky;Dilworth & Barrese, 333 Earle Ovington Boulevard, Uniondale, NY, 11553, US

(21) Appl. No.: 10/311,275

(22) PCT Filed: Jun. 15, 2001
(86) PCT No.: PCT/EP01/06771

Related U.S. Application Data
(60) Provisional application No. 60/217,603, filed on Jul. 11, 2000.

(30) Foreign Application Priority Data
Jun. 16, 2000 (EP) 00870133.6

(51) Int. Cl. C12N 15/82; A01H 5/00; C12N 15/29; C12Q 1/68

(52) U.S. Cl. 800/290; 536/23.6; 435/320.1; 435/252.3; 530/370; 530/387.1; 800/278; 800/298; 800/279; 435/6

(57) ABSTRACT

The present invention relates to novel plant type D cyclins, nucleic acid sequences encoding novel plant type D cyclins as well as to vectors, host cells, transonic cells and plants comprising said sequences. The invention also relates to methods for modifying cell fate and/or plant development and/or plant morphology and/or plant biochemistry and/or plant physiology comprising modifying the expression of plant type D cyclins or comprising the use of nucleic acid sequences encoding novel plant type D cyclins. The inventions also relates to methods for obtaining enhanced growth, and/or increased yield and/or delayed senescence of a plant cell, tissue and/or organ and/or increased frequence of formation of lateral organs in a plant, comprising the ectopic expression of a plant type D-cyclin. The invention also relates to methods for identifying and obtaining compounds interacting with plant type D cyclins. The invention also relates to the use of said compounds as a plant growth regulator or herbicide.

### FIELD OF THE INVENTION

The present invention relates to the field of new plant cyclin genes, proteins encoded thereby, derivatives thereof, transonic plants comprising said genes, as well as methods for modifying for instance plant growth and/or development.

### BACKGROUND TO THE INVENTION

Introduction to the Cell Cycle.

When eukaryotic cells, and thus also plant cells, divide they go through a highly ordered sequence of events collectively termed as the ‘cell cycle’. Briefly, DNA replication or synthesis (S) and mitotic segregation of the chromosomes (M) occur with intervening gap phases (G1 and G2) and the phases follow the sequence G1-S-G2-M. Cell division is completed after cytokinesis, the last step of the M-phase. Cells that have exited the cell cycle and have become quiescent are said to be in the G0 phase. Cells at the G0 stage can be stimulated to reenter the cell cycle at the G1 phase. The transition between the different phases of the cell cycle are basically driven by the sequential activation/inactivation of a kinase, denominated by ‘cyclin-dependent kinase) or Cdk, by different agonists. At least required for kinase activation are proteins called cyclins which also are important for targeting the kinase activity to a given (subset of) substrate(s). Other factors regulating Cdk activity include Cdk inhibitors (CKIs or ICKs, Kips, Cips, Inks), Cdk activating kinase (CAK), Cdk phosphatase (Cdc25) and Cdk subunit (CKS) (Mironov et al. 1999, Reed 1996 for reviews).

The Cell Cycle in Plants and the Roles of D-type Cycling Therein

In plants, and compared to animals, much less information on cell cycle mechanisms is available. Alongside unclassified Cdks, a number of plant Cdks has been classified in two types. A-type plant Cdks complement yeast Cdc28 mutants and are expressed in cyclins cells as well as in cells showing competence for division and are characterized by a PSTAIRE cyclin-binding motif. The closest mammalian homologues are Cdk1/Cdc2 and Cdk2. B-type plant Cdks have characteristic PPTALRE or PPTTLRE cyclin binding motifs and do not complement yeast Cdc28 mutants. No mammalian homologue of plant B-type Cdks is presently known. Arabidopsis A-type Cdks are constitutively expressed during the cell cycle and their kinase activity only markedly drops during G1. Expression of A-type Cdks is linked with competence for cell division. B-type Cdk genes are preferentially expressed during S and G2 and their activation starts at mid-G2, peaks at M and declines during G1 of the subsequent cell division (Ferreira et al. 1991, Hemerly et al. 1993, Hirayama et al. 1991, Mironov et al. 1999, Segers et al. 1996). A similar pattern is seen for the alfalfa (Medicago sativa) Cdks (Bögre et al. 1997, Magyar et al. 1997). Two snapdragon (A. majus) A-type Cdks are constitutively expressed in actively dividing cells whereas transcripts of the two B-type Cdks reside in isolated cells of dividing regions of the inflorescence (Fobert et al. 1996).

The plant D-type cyclin cDNAs from A. thaliana have been isolated by their ability to rescue cell cycle progression in G1 cyclin-deficient yeast (Soni et al. 1995). They were originally denominated as δ-cyclins but later renamed as cyclins D1, D2 and D3 (Renaudin et al. 1996). D-type cyclins have also been characterized in a number of other plants including Antirrhinum majus, Helianthus tuberosus, Medicago sativa and Zea mays (Dahl et al. 1995, Lowe et al. 2000—WO0017364, Murray et al. 1998—WO9842851). Cyclin D1 expression in Arabidopsis is most abundant in flowers and leaves but is virtually undetectable in liquid cultured calli or cell suspensions. Cyclin D2 expression is predominant in leaves and roots and can be induced by addition of sucrose, but not of phytohormones or nitrate, to starved cell suspensions. Cyclin D2 is subsequently constitutively expressed during the cell cycle. Constitutive expression of the Arabidopsis cyclin D2 in tobacco results in an increased growth rate (higher stems, larger leaves, larger flowers and flower number, larger number of seed pots, enhanced root development) whereas antisense expression of the same cyclin results in a decreased growth rate (Cockcroft et al. 2000, Murray et al. 1998—WO9842851). The highest expression of cyclin D3 is found in Arabidopsis roots and can in starved cell suspensions be triggered at G1/S by addition of sucrose, nitrate, as well as by cytokinin. Levels of cyclin D3 transcripts rise during S-phase and remain constant thereafter. Constitutive expression of cyclin D3 allows cell division to occur in the absence of exogenous cytokinin. Tobacco plants constitutively expressing Arabidopsis cyclin D3 have larger flowers which, however, develop later than in wild-type tobacco plants (Murray et al. 1998). Arabidopsis cyclin D4 is expressed during vascular tissue development, embyrogenesis and during formation of lateral roots primordia. Sucrose alone or in combination with phytohormones induces cyclin D4 expression in starved cell suspensions (Dahl et al. 1995, De Veyider et al. 1999, Ferreira et al. 1994, Fuerst et al. 1996, Inzé et al. 1999—WO9922002, Murray et al. 1998—WO9842851, Renaudin et al. 1996, Riou-Khamlichl et al. 1999, Soni et al. 1995).

Expression of a cyclin D3;2 gene is induced at G1 in tobacco cells re-entering the cell cycle from stationary phase cultures and remains at a constant level during the cell cycle. Two other cyclin D genes, cyclin D3;1 and cyclin D2;1 are surprisingly expressed during mitosis of synchronously dividing tobacco cells (Sorrell et al. 1999).

Snapdragon seedlings show an enhanced cyclin D1 gene expression when growing on media containing either sucrose, cytokinin or auxin or, surprisingly, also on media supplemented with eiter abscisic acid or brassinosteroids. Cyclin D1 transcripts are found throughout whole snapdragon plants. D3-type cyclin expression is localized to specific regions of all snapdragon shoot meristems. Expression of the genes encoding the two cyclin D3 isoforms is differentially induced in snapdragon seedlings by cytokinin or sucrose (cyclin D3a) and by sucrose (cyclin D3b) and repressed by abscisic acid or brassinosteroids (Gaudin et al. 2000).

All D-type plant cyclins contain, similar to their animal counterparts, an amino-terminal LxCxE (x=any amino acid) motif implied in the binding of the Rb pocket protein. Other characteristics common to plant and animal D-type cyclins are the cyclin box and the PEST sequences (De Veylder et al. 1999, Gaudin et al. 2000, Huntley et al. 1998, Inzé et al. 1999—WO9922002, Murray et al. 1998—WO9842851, Sorrell et al. 1999). PEST regions are rich in proline (P), aspartate or glutamate (D/E), serine (S) and threonine (T) and are usually flanked by positively charged amino acids. Proteins containing PEST sequences are rapidly degraded (Rogers et al. 1986).

In animals the principal target of cyclin D-Cdk4/6 or cyclin E-Cdc2 complexes is the retinoblastoma protein (Rb) which in its hypophosphorylated state inhibits the E2F-DP family of heterodimeric transcription factors. Activation of E2F-DPs is necessary to boost expression of genes of which the products are required to initiate S-phase (Müller and Helin 2000). To date, no E-type cyclins have been reported in plants. Plant Rb-homologues interacting with plant D-cyclins and/or with the wheat dwarf geminiviral RepA protein have been isolated from maize (Ach et al. 1997, Grafi et al. 1996, Huntley et al. 1998, Xie et al. 1995, 1996) and tobacco (Nakagami et al. 1999). The tobacco Rb protein is moreover phosphoraated by a Cdc2cyclin D kinase complex (Nakagami et al. 1999). A tobbaco E2F protein most similar to mammalian E2Fs 1 to 3 but lacking the cyclin A-binding domain has recently been characterized and been shown to interact with the tobacco Rb-homologue (Sekine et al. 1999). A carrot E2F protein is able to associate with a mammalian DP subunit and this complex acts as a transcriptional activator in both plant and mammalian cells (Albani et al. 2000).

The promoter of the Arabidopsis cyclin A2;1 gene drives cell cycle regulated reporter gene expression in cultured tobacco cells: expression is very low during G1, increases during S-phase with a peak at G2 and G2/M transition and decreases during M-phase. In the same experimental setup the Arabidopsis cyclin B1;1 promoter is activated upon S-phase exit with a peak at G2/M and during mitosis. The promoter is switched of after M-phase exit (Mironov et al. 1999, Shaul et al. 1996). Expression of the cyclin B1;1 in Arabidopsis plants is confined to dividing cells in root and shoot apical meristems and during embryogenesis (Ferreira et al. 1994). In rice roots cyclin A1;1 is expressed from early G2 till early M-phase while cyclin B2;1 and cyclin B2;2 transcripts accumulate from mid-G2 till the end of mitosis (Umeda et al. 1999). A- and B-type cyclins have been isolated from numerous other plants including alfalfa, maize, soybean and tobacco (Renaudin et al. 1998, Mironov et al. 1999).

As in animals, expression of most cyclin D genes in plants is induced upon perception of mitogenic signals such as sucrose and cytokinin, and plant D cyclins are required for initiation of the S-phase. Unlike in animals, however, expression of said D-cyclins in plants continues during the remainder of the cell cycle whereas expression of other cyclin D genes peaks during M-phase. The significance of cyclin D gene expression during M-phase remains elusive. Cycling D are therefor considered as G1-specific cyclins (Sorrell et al. 1999).

Surprisingly, transonic tobacco plants constitutively expressing either Arabidopsis cyclin D2 or cyclin D3 display different phenotypes, indicative of different functions of each type of cyclin D in the plant cell cycle. The different expression patterns of cyclin D genes, even within a given type, also points at specific functions of individual plant D-cyclins in the plant cell cycle. Any novel cyclin D-type thus provides unique ways of modifying plant growth and/or development.

Thus the technical problem underlying the present invention is to provide new cyclin D type molecules that are particularly useful in agriculture and plant cell and tissue culture. The solution to the technical problem is achieved by providing the embodiments characterized in the claims.

### SUMMARY OF THE INVENTION

In the present invention, a gene was isolated from Medicago truncatula (barrel medic) which encodes a novel type of D-cyclin. This cyclin is furtheron denominated MtCycDm.

Unexpectedly, the inventors discovered that the MtCycDm protein associates most strongly with Cdc2MsF, a B-type mitosis-specific cyclin-dependent kinase of Medicago sativa. Said MtCycDm furthermore interacts with the B-type Cdk Cdc2MsD active during G2-M and with the A-type Cdk Cdc2MsA active during both G1/S and G2/M transition in cyclins alfalfa cells. Both MtCycDm and Cdc2MsF, Cdc2MsD and Cdc2MsA are furthermore co-expressed during the cell cycle. Furthermore, out of a number of different Cdc2-cyclin complexes, the kinase activity of the Cdc2MsF-MtCycDm complex is most strongly inhibited by the barrel medic cyclin-dependent kinase inhibitor (CKIMt). The surprising interactions and/or co-expression of a D-cyclin with both G2-M-phase specific Cdks, G1/S specific Cdks and CKIs opens unprecedented avenues to modify plant growth and/or development and is clearly advantageous over the limited functions during the cell cycle of the D-type cyclins presently known in the art.

Accordingly the invention embodies an isolated DNA sequence with nucleotide sequence as given in SEQ ID NO 1, encoding a cell cycle control protein with amino acid sequence as given in SEQ ID NO 2, which is capable of interacting with other cell cycle control proteins comprising cyclin-dependent protein kinases. More specifically, said isolated DNA sequence encodes a plant cyclin D of a novel type which is unexpectedly able to interact with an M-phase B-type PPTTLRE Cdc2 kinase comprised within the group of cyclin-dependent kinase cell cycle control proteins. Said novel plant cyclin D is furthermore capable of interaction, though less pronounced, with a B-type PPTALRE Cdc2 kinase comprised within the group of cyclin-dependent kinase cell cycle control proteins and involved in at least the G2- to M-phase transition. The interaction of said novel plant cyclin D with A-type PSTAIRE Cdc2 kinases comprised within the group of cyclin-dependent kinase cell cycle control proteins is unexpectedly weak or insignificant. Said novel plant cyclin D furthermore interacts with the barrel medic CKI and this CKI most strongly inhibits kinase activity of the complex formed by a PPTTLRE Cdc2 and the cyclin D of the invention.

A preferred embodiment of the current invention comprises an isolated nucleic acid encoding a novel plant type D-cyclin or encoding an immunologically active and/or functional fragment of such a protein selected from the group consisting of:
(a) nucleic acids comprising at least part, preferably at least 1500, 1250, 1000 or 750 nucleotides, most preferably at least 500, 450, 400, 350, 300, 250, 200, 150,100, 75 or 50 nucleotides, most preferably at least 45, 41, 40, 35, 30, 25 or 20 nucleotides of the DNA sequence as given in SEQ ID NO 1, or the complement thereof,
(b) nucleic acids comprising the RNA sequences corresponding to at least part of SEQ ID NO 1, or the complement thereof,
(c) nucleic acids specifically hybridising with the nucleotide sequence as defined in any one of SEQ ID NOs 1 or 16 to 21,
(d) nucleic acid sequences encoding a protein with an amino acid sequence which is at least 55% identical, preferably 60%, 65%, 70% or 75% identical, more preferable 80%, 85% or 90% identical, most preferable 95% identical to the amino acid sequence as given in SEQ ID NO 2,
(e) nucleic acids encoding a protein comprising the amino acid sequence as given in any of SEQ ID NOs 2 to 6 or 28 to 36,
(f) nucleic acids which are degenerated as a result of the genetic code to a nucleic acid encoding a protein as given in SEQ ID NO 2 or as defined in (a) to (e),
(g) nucleic acids which are diverging due to the differences in codon usage between the organisms to a nucleic acid encoding a protein as given in SEQ ID NO 2 or to a nucleic acid as defined in (a) to (e),
(h) nucleic acids which are diverging due to the differences between alleles encoding a protein as given in SEQ ID NO 2 or as defined in (a) to (e),
(i) nucleic acids encoding a fragment, preferably of at least 300, 250, 200, 150 or 100 amino acids of a protein as given in SEQ ID NO 2, more preferably encoding a fragment of at least 90, 80 or 70 amino acids of a protein as given in SEQ ID NO 2 or 28, more preferably encoding a fragment of at least 60, 50, 40 or 30 amino acids of a protein as given in any of SEQ ID NOs 2, 28, 30 or 34, most preferably ecoding a fragment of at least 15 amino acids of a protein as given in any of SEQ ID NOs 2, or 28 to 36,
(j) nucleic acids specifically hybridizing to a nuclelc acid encoding a peptide as given in any of SEQ ID NOs 3 to 6 or 28 to 36, and
(k) nucleic acid sequences encoding a protein as defined in SEQ ID NO 2 or as defined in any one of (a) to (j) interrupted by intervening DNA sequences.

Another related embodiment includes DNA sequences encoding functional plant D-cyclins comprising one or more protein regions distinguishing said plant D-cyclin from those plant D-cyclins known in the art identified during the work leading to the present invention to be most closely related to the cyclin D of the invention. Said protein regions include the plurality of amino acid sequence features specifically distinguishing the amino acid sequence defined in SEQ ID NO 2 from the amino acid sequences of plant D1- and D2-type cyclins and selected from the group consisting of:
(a) unique regions in the cyclin box of the cyclin D as defined in SEQ ID NO 2 including SEQ ID NO 3 and SEQ ID NO 4; and
(b) additional unique regions present in the cyclin D as defined in SEQ ID NO 2 including SEQ ID NO 5; and
(c) unique longer carboxy-terminal peptide of the cyclin D as defined in SEQ ID NO 2 represented by SEQ ID NO 6; and
(d) peptides at least 70% identical, preferably 75% or 80% identical, more preferable 85% or 90% identical, most preferable 95% to the amino acid sequences as defined in any of (a) to (d).

A further embodiment of the invention comprises homologues, derivatives and/or immunologically active fragments of D-type cyclins according to the invention, fragments thereof and proteins comprising said homologues, derivatives and/or immunologically active fragments of said D-type cyclins or fragments thereof.

The current invention furthermore encompasses antisera and/or antibodies specifically recognizing the D-type cyclin according to the invention or immunologically active parts thereof.

In another embodiment of the invention, a method is provided for modifying cell fate and/or plant development and/or plant morphology and/or biochemistry and/or physiology comprising the modification of expression in particular cells, tissues or organs of a plant, of a nucleic acid sequence as defined above operably in connection with a plant-operable promoter sequence.

Provided in the current invention are methods to effect expression a cyclin D protein or a homologue or derivative thereof as defined in the current invention in a plant cell,. tissue or organ.The present invention clearly extends to any plant produced by the inventive method described herein.

The present invention further relates to a method for identifying and obtaining agonists of a cyclin D protein or a homologue or derivative thereof as defined in the current invention and/or of the interaction of said cyclin D with Cdc2-type or Cdc2-related kinases.

### DETAILED DESCRIPTION OF THE INVENTION

Aspects and Preferred Embodiments of the Invention

A first aspect of the invention comprises the nucleotide sequence of a novel D-type cyclin gene isolated from M. truncatula. Said gene, termed MTCYCDM furtheron, is listed in the current specification as SEQ ID NO 1. The isolation of MTCYCDM is described in Example 1.

A second aspect of the invention comprises the amino acid sequence of the novel D-type cyclin encoded by said MTCYCDM gene. Said sequence of the MtCycDm protein is listed in the current specification as SEQ ID NO 2. The classification of MtCycDm as a novel type of D-cyclin is extensively documented in FIGS. 1 through 5 and in Tables 1 and 2 which are decrypted in the discussion following infra.

An initial comparative amino acid sequence homology search using the BLASTP 2.0.5 software (Altschul et al., 1997) revealed that the most significant alignments of MtCycDm are produced with D-type plant cyclins and, more specifically, with D1- and D2-type plant cyclins (Table 1).

TABLE 1
Sequences producing significant alignment with MtCycDm
as identified using the BLASTP 2.0.5 software (Altschul et al. 1997).
The five sequences most homologous to MtCycDm are listed.

| Sequence | | | |
|---|---|---|---|
| accession | | Score | |
| number | Sequence description | (bits) | E-value |
| AC002062 | Strong similarity to Arabidopsis | 258 | 6e−68 |
| | thaliana cyclin D1 | | |
| P42751 | Arabidopsis thaliana cyclin D1 | 257 | 1e−67 |
| AJ250396 | Anthirrhinum majus cyclin D1 | 244 | 1e−63 |
| S51650 | Arabidopsis thaliana cyclin D1 | 238 | 7e−62 |
| S51651 | Arabidopsis thaliana cyclin D2 | 131 | 1e−29 |

The “E-value” (Expect value) herein mentioned is a parameter that describes the number of hits one can “expect” to see just by chance when searching a database of a particular size. The more complex the database, the greater the chance that it decreases exponentially with the Score (S) that is assigned to a match between two sequences. Essentially, the E value describes the random background noise that exists for matches between sequences. The Expect value is used as a convenient way to create a significance threshold for reporting results. When the Expect value is increased from the default value of 10, a larger list with more low-scoring hits can be reported. In BLAST 2.0, the Expect value is also used instead of the P value (probability) to report the significance of matches. For example, an E value of 1 assigned to a hit can be interpreted as meaning that in a database of the current size one might expect to see 1 match with a similar score simply by chance.

An amino acid sequence alignment of plant D1 and D2 cyclins from A. thaliana, A. majus, Chenopodium rubrum, Nicotiana tabacum, Zea mays and MtCycDm is represented in FIG. 1. Careful analysis of this alignment learns that MtCycDm has a plurality of amino acid sequence features clearly distinguishing it from D1- and D2-type plant cyclins. Said features include:
(i) unique regions in cyclin box: residues 170-185 according to the amino acid numbering in FIG. 1 (WMATATFICCMFVFSS; SEQ ID NO 3, amino acid residues 133-148 of MtCycDm) and residues 187-190 according to the amino acid numbering in FIG. 1 (DGGP; SEQ ID NO 4, amino acid residues 150-153 of MtCycDm); and
(ii) additional unique regions: residues 212-214 according to the amino acid numbering in FIG. 1 (MLI, amino acid residues 175-177 of MtCycDm), residues 232-246 according to the amino acid numbering in FIG. 1 (HLVSSVSLRATRF; SEQ ID NO 5, amino acid residues 195-207 of MtCycDm); and
(iii) unique longer carboxy-terminal peptide: residues 404-412 according to the amino acid numbering in FIG. 1 (GREKKQTR; SEQ ID NO 6, amino acid residues 351-359 of MtCycDm).

The novelty of the MtCycDm protein sequence relative to known plant D1- and D2-type cyclins is further substantiated in phylogenetic tree analyses. Such an analysis using the PROTDIST software (Felsenstein 1993; http://evolution.genetics.washington.edu/phylip.html) and perfomed on the full-length protein sequences reveals that MtCycDm localizes on the D1-type plant cyclin branch (FIG. 2) which is consistent with the BLASTP results (Table 1). It should, however, be noted that MtCycDm branches far out from the A. thaliana, A. majus and H.tuberosus D1 cyclins, i.e. MtCycDm is significantly different from said other plant D1 cyclins. The significance of the difference between MtCycDm and plant D1-cyclins is largely increased when a phylogenetic tree is drawn for part of the plant cyclin protein sequences, more specifically the cyclin box region. The cyclin box is a region of ˜100 amino acids which is present at the amino-terminus of all known cyclin and which confers to cyclins the capacity to bind cyclin-dependent kinases, a prerequisite for Cdk activation. Differences between cyclin boxes of cyclins are expected to confer specificity in cyclin-Cdk interactions and to affect the strength of cyclin-Cdk association.

The cyclin box is indicated in FIG. 1 and the cyclin box of the MtCycDm protein of the invention is defined in SEQ ID NO 28. Part of said cyclin box corresponds to the PROSITE (Hofmann et al. 1999) Cyclins Consensus Pattern (PS00292) which is defined by “R-x(2)-[LIVMSA]-x(2)-[FYWS]-[LIVM]-x(8)-[LIVMFC]-x(4)-[LIVMFYA]-x(2)-[STAGC]-[LIVMFYQ]-x-[LIVMFYC]-[LIVMFY]-D-[RKH]-[LIVMFYW]” wherein x is any amino acid, x(N) is a stretch of N contiguous amino acids and wherein e.g. [LIVM] means that at the given position either of the listed amino acid residues, i.e. Leu, lle, Val and Met, is preferentially occurring. The part of the MtCycDm cyclin box corresponding to the PROSITE PS00292 Cyclin Consensus Pattern is defined in SEQ ID NO 29.

FIG. 3 depicts the cyclin box-based phylogenetic tree of different plant cyclins. Again MtCycDm localizes to the D1-type plant cyclin branch but the distance between MtCycDm and D1-cyclins of A. thaliana, A. majus and H. tuberosus is even far more pronounced as in the case of the whole protein sequences (compare FIG. 2 and FIG. 3).

Although MtCycDm is situated on the D1-type plant cyclin branch, its molecular weight is significantly higher than that of D1-type plant cyclins and falls in the range of the molecular weights of D2-type plant cyclins (Table 2). This observation further adds to the distinction between D1-type plant cyclins and MtCycDm.

TABLE 2
List of plant cyclins D with their molecular weights and
number of amino acid sequences. Am: Anthirrhinum majus;
At: Arabidopsis thaliana; Cr: Chenopodium rubrum;
Ht: Helianthus tuberosus; Le: Lycopersicon esculentum;
Ms: Medicago sativa; Mt: Medicago truncatula;
Nt: Nicotiana tabacum; Zm: Zea mays.

| | | Number of |
|---|---|---|
| Name | MW | amino acids |
| NtcycD3; 2 | 41611.74 | 367 |
| NtcycD3; 1 | 42977.84 | 373 |
| LecycD3; 3 | 38685.10 | 336 |
| LecycD3; 2 | 41788.80 | 364 |
| LecycD3; 1 | 40968.89 | 359 |
| MscycD3; 1 | 44054.50 | 386 |
| AtcycD3; 1 | 42824.79 | 368 |
| AmcycD1 | 36492.04 | 330 |
| AtcycD1; 1 | 37866.01 | 335 |
| HtcycD1; 1 | 35649.66 | 315 |
| MtcycDm | 40424.63 | 359 |
| AtcycD4; 1 | 34685.91 | 308 |
| AtcycD2; 1 | 40577.50 | 361 |
| NtcycD2; 1 | 39712.52 | 354 |
| CrcycD2; 1 | 41766.51 | 372 |
| ZmcycD2 | 38834.23 | 358 |

Calculation of the percentage of identical amino acid residues between MtCycDm and plant D1- and D2-type cyclins indicates that MtCycDm is only 29 to 55% identical to D1- or D2-type plant cyclins (Table 3). Within the cyclin box region, D1-type and D2-type plant cyclins are 73 to 75% and 64 to 75% identical to each other, respectively. The cyclin box of MtCycDm is, however, only 35 to 54% identical to the cyclin boxes of D1- or D2-type plant cyclins (Table 4). The percentage data were obtained by pairwise alignments of the sequences using the GAP program of the GCG program package (Wisconsin Package, Version 8, September 1994, Genetics Computer Group, 575 Science Drive, Madison, Wis., USA 53711) with default parameter settings (gap penalty=3.0; gap length penalty=0.1).

TABLE 3
Percentage Identity between plant cyclin D
amino acid sequences. Am: Anthirrhinum
majus; At: Arabidopsis thaliana;
Cr: Chenopodium rubrum;
Ht: Helianthus tuberosus; Mt: Medicago
truncatula; Nt: Nicotiana tabacum; Zm:
Zea mays.

| | AtcycD1; 1 | AmcycD1 | HtcycD1; 1 | MtcycDm | AtcycD4; 1 | AtcycD2; 1 | NtcycD2; 1 | CrcycD2; 1 |
|---|---|---|---|---|---|---|---|---|
| AtcycD1; 1 | * | | | | | | | |
| AmcycD1 | 36 | * | | | | | | |
| HtcycD1; 1 | 60.7 | 63.9 | * | | | | | |
| MtcycDm | 52.7 | 46.5 | 54.7 | * | | | | |
| AtcycD4; 1 | 40.3 | 34.9 | 36.4 | 30 | * | | | |
| AtcycD2; 1 | 37.5 | 34.4 | 37 | 35 | 53.7 | * | | |
| NtcycD2; 1 | 36 | 36.9 | 38.3 | 28.8 | 53.9 | 52.7 | * | |
| CrcycD2; 1 | 44.3 | 37.3 | 45.5 | 31.7 | 50.8 | 46.2 | 55.9 | * |
| ZmcycD2 | 36.7 | 36.2 | 39.1 | 31.8 | 43.6 | 44.4 | 48.8 | 48.1 |

TABLE 4
Percentage identity between the cyclin
box regions of plant cyclin D
amino acid sequences. Am:
Anthirrhinum majus; At:
Arabidopsis thaliana; Cr:
Chenopodium rubrum;
Ht: Helianthus tuberosus; Mt:
Medicago truncatula; Nt:
Nicotiana tabacum; Zm: Zea mays.

| | AtcycD1; 1 | AmcycD1 | HtcycD1; 1 | MtcycDm | AtcycD4; 1 | AtcycD2; 1 | NtcycD2; 1 | CrcycD2; 1 |
|---|---|---|---|---|---|---|---|---|
| AtcycD1; 1 | * | | | | | | | |
| AmcycD1 | 72.7 | * | | | | | | |
| HtcycD1; 1 | 73.7 | 74.7 | * | | | | | |
| MtcycDm | 53.5 | 53.5 | 53.5 | * | | | | |
| AtcycD4; 1 | 55.5 | 54.5 | 50.5 | 35.4 | * | | | |
| AtcycD2; 1 | 56.5 | 52.2 | 54.5 | 42.4 | 62.6 | * | | |
| NtcycD2; 1 | 57.6 | 55.5 | 56.6 | 40.4 | 66.7 | 70.7 | * | |
| CrcycD2; 1 | 62.2 | 59.2 | 60.2 | 44.8 | 62.2 | 68.4 | 69.3 | * |
| ZmcycD2 | 61.6 | 56.6 | 59.6 | 46.5 | 60.6 | 63.6 | 67.7 | 74.5 |

Further distinction between MtCycDm and plant D1- and D2-type cyclins is clearly apparent when analyzing the amino acid sequences for the presence of PEST-sequences using the PESTFIND software (downloadable from http://www.ebi.ac.uk/biocat/index.html). Such an analysis reveals the presence of an extensive PEST-region in the amino-terminus of MtCycDm and plant D1- and D2-type cyclins except in N. tabacum CycD2;1. An additional carboxy-terminal PEST region is comprised in all other D1- and D2-type plant cyclins with the exception of MtCycDm and N. tabacum CycD2;1. (FIG. 4). The unique PEST region of the MtCycDm protein of the invention is defined by SEQ ID NO 30. Common to D-type cyclins in general and to plant D-type cyclins more specifically, MtCycDm contains the LxCxE (x=any amino acid; LLCGE; SEQ ID NO 7, amino acid residues 18-22 of MtCycDm) enabling interaction with pocket proteins such as Rb. No consensus Cdk-phosphorylation sites {TPX1-2[R/K] or SPX[R/K] or SPX(Hu) or SP(Hu)X, with X=any amino acid and Hu=hydrophobic uncharged amino acid [M/I/L/V]; based on Tamrakar et al 2000} are apparent in the MtCycDm amino acid. In the MtCycDm protein are furthermore comprised two repeats of a region consisting of 5 consecutive serine residues (amino acid residues 41-45 and 321-325 of MtCycDm).

The presented data thus clearly distinguish MtCycDm from other plant D-type cyclins and, more specifically, from plant D1- and D2-type cyclins which represent the closest homologs of MtCycDm.

A further aspect of the current invention includes the unexpected interaction patterns of MtCycDm with cyclin-dependent kinases. In vivo interaction between proteins can be easily studied using the yeast two-hybrid assay. When applying the yeast two-hybrid assay to different Medicago D-type cyclins and Cdks, the specific association of MtCycDm with Cdks active during G2-M was established as described in Example 2. The results of the conducted experiments are depicted in FIG. 5. The strongest interaction occurs between MtCycDm and Cdc2MsF followed by the MtCycDm-Cdc2MsD and MtCycDm-Cdc2MsA associations. The strength of the MtCycDm-Cdc2MsB interaction is not convincingly high. Significantly, cyclin D3;1 does not interact with any of the G2-M-active alfalfa Cdks. The observed binding of MtCycDm to both B-type Cdks Cdc2MsD and Cdc2MsF and, less pronounced, to A-type Cdk Cdc2MsA is unprecedented, sustains the importance of the cyclin box in determining specificity in cyclin-Cdk interactions and raises the question of the biological relevance of this interaction. The novelty of these observations is further sustained by the recent documentation of the A. thaliana cyclin D2 binding with the tobacco A-type, but not B-type, Cdc2-type or Cdc2-related kinase (Cockcroft et al., 2000)

Therefore, in another aspect of the invention, experiments were conducted to establish whether or not MtCycDm and Cdc2MsA/D/F are co-expressed during the cell cycle. Using Northern blot analysis and RT-PCR analysis (see Examples 3 and 4, respectively) it was obviated that the MtCycDm and Cdc2MsF genes are indeed co-expressed during the cell cycle (FIGS. 6 and 7). Levels of MtCycDm mRNA peak at late S-phase, subsequently decline, peak again during M-phase, stay constant and start to increase again at late G1. Cdc2MsF mRNA levels rise at the onset of G2 and start to decrease at late M-phase. An overview of Cdc2MsA/B/D/F kinase activities (see Example 5) is given in FIG. 8, clarifying that Cdc2MsA/B, Cdc2MsD and Cdc2MsF are successively active during G2-M. It should be noted that previous experiments have shown that Cdc2MsA/B activity additionally peaks at the G1-S transition and that Cdc2MsF is exclusively active during G2-M (Magyar et al. 1997). The activity of Cdc2MsA/B in both S-phase and during G2-M is also illustrated in FIG. 15.

A further unexpected aspect of the current invention includes the observation that the cyclin-dependent kinase inhibitor of Medicago (hereinafter referred to as CKIMt) is capable of differentially modulating protein kinase activities of different MtCycDm-Medicago Cdc2 complexes. First, said CKIMt is in a yeast two-hybrid system, interacting strongly with Cdc2MsA and MtCycDm (FIGS. 12 and 13, respectively). CKIMt further interacts with Cdc2MsB and Cdc2MsC, but not with Cdc2MsF (FIG. 12); and with a cyclin D3 and a cyclin D5 (FIG. 13). Surprisingly, protein kinase activity is inhibited by CKIMt in protein complexes containing Cdc2MsF and Cdc2MsA/B (FIGS. 14 and 15, respectively) but activated by CKIMt in protein complexes containing Cdc2MSD (FIG. 14). As CKIMt is only very weakly interacting with Cdc2MsF and Cdc2MsD, the protein kinase inactivation and activation, respectively, by CKIMt is likely to be conveyed via MtCycDm which interacts with both Cdc2MsF and Cdc2MsD. CKIMt moreover most strongly inhibits histone H1 kinase activity of the Cdc2MsF-MtCycDm complex.

Combining the protein interaction and gene expression data indicates that the novel plant D-type cyclin of the invention, MtCycDm, is very likely to exert a previously unrecognized function during the G2 and M phases of the plant cell cycle via interaction with Cdc2MsA, Cdc2MsD and Cdc2MsF. MtCycDm might furthermore exert its ‘expected cyclin D-function’ via interaction with Cdc2MsA during G1-S transition. In addition, the function of MtCycDm during G2-M phase of the plant cell cycle can be modulated via interaction of MtCycDm with the barrel medic CKIMt.

In a further aspect of the invention the distribution of the Cdc2MsF kinase protein was analyzed by Immunolocalization (Example 6). Typical results of this experiment are shown in FIG. 9 and situate the Cdc2MsF protein to the preprophase band, prophase spindle, metaphase spindle and phragmoplast. Said Cdc2MsF localization pattern is indicative of a pivotal role for said kinase in mitotic plant cells and more specifically in determining cell plate formation, karyokinesis and cytokinesis.

Said aspects of the invention lead to the formulation of the following preferred embodiments of the current invention.

Accordingly the invention embodies an isolated DNA sequence with nucleotide sequence as given in SEQ ID NO 1, encoding a cell cycle control protein with amino acid sequence as given in SEQ ID NO 2, which is capable of interacting with other cell cycle control proteins comprising cyclin-dependent protein kinases. More specifically, said isolated DNA sequence encodes a plant cyclin D of a novel type which is unexpectedly able to interact with an M-phase B-type PPTTLRE Cdc2 kinase comprised within the group of cyclin-dependent kinase cell cycle control proteins. Said novel plant cyclin D is furthermore capable of interaction, though less pronounced, with a B-type PPTALRE Cdc2 kinase comprised within the group of cyclin-dependent kinase cell cycle control proteins and involved in at least the G2- to M-phase transition. The interaction of said novel plant cyclin D with A-type PSTAIRE Cdc2 kinases comprised within the group of cyclin-dependent kinase cell cycle control proteins is unexpectedly weak or insignificant. Said novel plant cyclin D furthermore interacts with the barrel medic CKI and this CKI most strongly inhibits kinase activity of the complex formed by a PPTTLRE Cdc2 and the cyclin D of the invention.

A related preferred embodiment of the current invention comprises an isolated nucleic acid encoding a novel plant type D-cyclin or encoding an immunologically active and/or a functional fragment of such a protein selected from the group consisting of:
(a) nucleic acids comprising at least part, preferably at least 1500, 1250, 1000 or 750 nucleotides, most preferably at least 500, 450, 400, 350, 300, 250, 200, 150, 100, 75 or 50 nucleotides, most preferably at least 45, 41,40, 35, 30, 25 or 20 nucleotides of the DNA sequence as given in SEQ ID NO 1, or the complement thereof,
(b) nucleic acids comprising the RNA sequences corresponding to at least part of SEQ ID NO 1, or the complement thereof,
(c) nucleic acids specifically hybridising with the nucleotide sequence as defined in any one of SEQ ID NOs 1 or 16 to 21,
(d) nucleic acid sequences encoding a protein with an amino acid sequence which is at least 55% identical, preferably 60%, 65%, 70% or 75% identical, more preferable 80%, 85% or 90% identical, most preferable 95% identical to the amino acid sequence as given in SEQ ID NO 2,
(e) nucleic acids encoding a protein comprising the amino acid sequence as given in any of SEQ ID NOs 2 to 6 or 28 to 36,
(f) nucleic acids which are degenerated as a result of the genetic code to a nucleic acid encoding a protein as given in SEQ ID NO 2 or as defined in (a) to (e),
(g) nucleic acids which are diverging due to the differences in codon usage between the organisms to a nucleic acid encoding a protein as given in SEQ ID NO 2 or to a nucleic acid as defined in (a) to (e),
(h) nucleic acids which are diverging due to the differences between alleles encoding a protein as given in SEQ ID NO 2 or as defined in (a) to (e),
(i) nucleic acids encoding a fragment, preferably of at least 300, 250, 200, 150 or 100 amino acids of a protein as given in SEQ ID NO 2, more preferably encoding a fragment of at least 90, 80 or 70 amino acids of a protein as given in SEQ ID NO 2 or 28, more preferably encoding a fragment of at least 60, 50, 40 or 30 amino acids of a protein as given in any of SEQ ID NOs 2, 28, 30 or 34, most preferably ecoding a fragment of at least 15 amino acids of a protein as given in any of SEQ ID NOs 2, or 28 to 36,
(j) nucleic acids specifically hybridizing to a nucleic acid encoding a peptide as given in any of SEQ ID NOs 3, 5, 6 or 28 to 36, and
(k) nucleic acid sequences encoding a protein as defined in SEQ ID NO 2 or as defined in any one of (a) to (j) interrupted by intervening DNA sequences.

The present invention also relates to isolated nucleic acids as mentioned above under (a) to (k) which are DNA, cDNA, genomic DNA or synthetic DNA, or RNA wherein T is replaced by U.

Functional fragments of the MtCycDm protein of the invention include its cyclin box, its PEST region, its LxCxE motif and its unique regions. Said functional fragments thus include the MtCycDm protein fragments as defined by SEQ ID NOs 3 to 7 and 28 to 30. Functional fragments of the MtCycDm protein also include fragments having the biological activity of a plant D-cyclin.

Immunologically active fragments of the MtCycDm protein of the invention comprise fragments as defined by SEQ ID NOs 31 to 110.

A functional fragment according to the invention can at the same time be an immunologically active fragment or not.

Another related embodiment includes DNA sequences encoding functional plant D-cyclins comprising one or more protein regions, distinguishing said plant D-cyclin from those plant D-cyclins known in the art, identified during the work leading to the present invention to be most closely related to the cyclin D of the invention. Said protein regions include the plurally of amino acid sequence features specifically distinguishing the amino acid sequence defined in SEQ ID NO 2 from the amino acid sequences of plant D1- and D2-type cyclins and selected from the group consisting of:
(a) unique regions in the cyclin box of the cyclin D as defined in SEQ ID NO 2 including SEQ ID NO 3 and SEQ ID NO 4; and
(b) additional unique regions present in the cyclin D as defined in SEQ ID NO 2 including SEQ ID NO 5; and
(c) unique longer carboxy-terminal peptide of the cyclin D as defined in SEQ ID NO 2 represented by SEQ ID NO 6; and
(d) peptides at least 70% identical, preferably 75% or 80% identical, more preferable 85% or 90% identical, most preferable 95% identical to the amino acid sequences as defined in any of (a) to (d).

Plant D1- and D2-type cyclins most closely related to the cyclin D of the invention identified by SEQ ID NO 2 include A. majus cyclin D1 (Gaudin et al 2000), A. thaliana cyclin D1;1 and D2;1 (GenBank accession numbers X83369 and X83370, respectively), C. rubrum (GenBank accesion number Y10162), Helianthus tuberosus (Murray et al. 1998—WO9842581), N. tabacum cyclin D2;1 (GenBank accession number AJ011892) and Zea mays cyclin D2 (Murray et al. 1998—WO9842581). Also part of the invention are nucleic acid molecules of at least 15 nucleotides in length hybridizing specifically with at least one of the nucleic acid molecules of the invention as defined above or specifically amplifying the above defined nucleic acid molecules. According to another embodiment, the invention relates to a vector comprising a nucleic acid sequence of the invention. This vector can be an expression vector wherein the nucleic acid sequence is operably linked to one or more control sequences allowing the expression in prokaryotic and/or eukaryotic host cells.

Also the host cell containing a nucleic acid molecule of the invention are part of the present invention. Preferred host cells according to the invention are bacterial, insect, fungal, plant or animal cells.

A further embodiment of the invention comprises homologues, derivatives and/or immunologically active fragments of D-type cyclins according to the invention, fragments thereof and proteins comprising said homologues, derivatives and/or immunologically active fragments of said D-type cyclins or fragments thereof.

As such, the present invention also relates to an isolated polypeptide encodable by a nucleic acid molecule of the invention as defined above, or a homologue or a derivative of said polypeptide, or an immunologically active and/or functional fragment thereof. In a more preferred embodiment, the invention relates to a polypeptide, encodable by a nucleic acid molecule of the invention and which has an amino acid sequence as given in SEQ ID NO 2, or a homologue or a derivative thereof, or an immunologically active and/or functional fragment thereof, preferably said immunologically active or functional fragment has an amino acid sequence as presented in any of SEQ ID NOs 3 to 6 or 28 to 110.

The invention further relates to the polypeptides as defined above which have the ability to form a complex with a cell-cycle dependent kinase or an inhibitor thereof (CKI). Preferrebly said cell-cycle dependent kinase is chosen from the group of plant B-type PPTTLRE and PPTALRE Cdc2 kinases and said cell-cycle dependent kinase Inhibitor (CKI) is the barrel medic CKI or a functional homologue thereof.

Therefore, according to another embodiment, the invention also relates to a method for producing a polypeptide of the invention comprising culturing a host cell further specified above under conditions allowing the expression of the polypeptide and recovering the produced polypeptide from the culture.

Alternatively, any of said proteins can be produced in a biological system, e.g. a cell culture. Alternatively any of said proteins is chemically manufactured e.g. by solid phase peptide synthesis. Said proteins or fragments thereof can be part of a fusion protein as is the case in e.g. a two-hybrid assay which enables e.g. the identification of proteins interacting with the cyclin D according to the invention other than the Cdc2-type or Cdc2-related kinases. An example of such other interacting protein is the barrel medic CKI as identified in the current invention.

The proteins or fragments thereof obtained by a method of the invention, e.g. the barrel medic CKI, are furthermore useful e.g. to modulate the interaction between a cyclin D according to the invention and Cdc2-type or Cdc2-related kinases and/or other identified interacting protein partners. Chemically synthesized peptides are particularly useful e.g. as a source of antigens for the production of antisera and/or antibodies.

The current invention thus furthermore encompasses antisera and/or antibodies specifically recognizing the D-type cyclin according to the invention or immunologically active parts or epitopes thereof. Said antisera and/or antibodies are useful in many areas related to the invention including: (i) identification in any organism, preferably plants, of other D-type cyclins and their genes according to the invention; (ii) quantification of synthesis in organisms and/or recombinant organisms of the cyclin D according to the invention; (iii) purification of the cyclin D according to the invention; (iv) immunoprecipitation of the cyclin D according to the invention e.g. as a way to identify other protein partners complexing with said cyclin D; (v) immunolocalization of the cyclin D according to the invention which is expressed in an organism or a recombinant organism.

As mentioned supra, the combined protein interaction and gene expression data indicates that the novel plant D-type cyclin of the invention, MtCycDm (SEQ ID NO 2), is very likely to exert a previously unrecognized function during the G2 and M phases of the plant cell cycle via interaction with Cdc2MsA, Cdc2MsD and Cdc2MsF. MtCycDm might furthermore exert its expected cyclin D-function via interaction with Cdc2MsA during G1-S transition. The unexpected pleiotropy of MtCycDm functions during the cell cycle opens unprecedented avenues to modify plant growth and/or development and is clearly advantageous over the limited functions during the cell cycle of the D-type cyclins presently known in the art. Said unexpected pleiotropic MtCycDm functions can furthermore be fine-tuned by the barrel medic CKI or a functional homologue thereof.

Therefore in yet other embodiments of the invention, methods are provided for modifying cell fate and/or plant development and/or plant morphology and/or biochemistry and/or physiology comprising the modification of expression in particular cells, tissues or organs of a plant, of a genetic sequence encoding a cell cycle control protein, and, preferrably a cyclin protein encoded by any nucleic acid of the invention operably linked to a plant-operable promoter sequence.

In a particularly preferred embodiment of the invention, the cyclin protein is a cyclin D protein according to the invention, preferably a plant cyclin D, and, more particularly, the barrel medic MtCycDm protein, or a biologically-active homologue or derivative thereof. The present invention clearly contemplates the use of functional homologues of D-type cyclins according to the present invention. Accordingly, the present invention is not limited in application to the use of nucleotide sequences encoding the barrel medic MtCycDm protein. It can be expected that genes and proteins similar to the one here defined from barrel medic are present in other plant species and can be isolated by means of techniques known in the art. These similar genes are also within the scope of the present invention.

Modulation of the expression in a plant of a cyclin D protein or a homologue or derivative thereof as defined in the current invention can produce a range of desirable phenotypes in plants, such as, for example, by modifying one or more morphological, biochemical, or physiological characteristics including: (i) modifying the length of the G1 and/or the S and/or the G2 and/or the M phase of the cell cycle of a plant; (ii) modifying the G1/S and/or S/G2 and/or G2/M and/or M/G1 phase transition of a plant cell; (iii) modification of the initiation, promotion, stimulation or enhancement of cell division; (iv) modification of the initiation, promotion, stimulation or enhancement of DNA replication; (v) modification of the initiation, promotion, stimulation or enhancement of seed set and/or seed size and/or seed development; (vi) modification of the initiation, promotion, stimulation or enhancement of tuber formation; (vii) modification of the initiation, promotion, stimulation or enhancement of fruit formation; (viii) modification of the initiation, promotion, stimulation or enhancement of leaf formation; (ix) modification of the initiation, promotion, stimulation or enhancement of shoot initiation and/or development; (x) modification of the initiation, promotion, stimulation or enhancement of root initiation and/or development; (xi) modification of the initiation, promotion, stimulation or enhancement of lateral root initiation and/or development; (xii) modification of the initiation, promotion, stimulation or enhancement of nodule formation and/or nodule function; (xiii) modification of the initiation, promotion, stimulation or enhancement of bushiness of the plant; (xiv) modification of the initiation, promotion, stimulation or enhancement of dwarfism in the plant; (xv) modification of the initiation, promotion, stimulation or enhancement of senescence; (xvi) modification of stem thickness and/or strength characteristics and/or wind-resistance of the stem and/or stem length; (xvii) modification of tolerance and/or resistance to biotic stresses such as pathogen infection; and (xviii) modification of tolerance and/or resistance to abiotic stresses such as drought stress or salt stress.

The present invention also relates to a method for the production of transonic plants plant cells or plant tissues comprising the introduction of a nucleic acid molecule of the invention in an expressible format or a vector as defined above in said plant, plant cell or plant tissue.

Methods to effect expression a cyclin D protein or a homologue or derivative thereof as defined in the current invention in a plant cell, tissue or organ, include either the introduction of the protein directly to said cell, tissue or organ such as by microinjection of ballistic means or, alternatively, introduction of an isolated nucleic acid molecule encoding said protein in an expressible format, stably into the genome of a plant cell. Methods to effect expression of a cyclin D protein or a homologue or derivative thereof as defined in the current invention in whole plants include regeneration of whole plants from said transformed cells in which an isolated nucleic acid molecule encoding said protein was introduced in an expressible format.

The present invention clearly extends to any plant cell or plant produced by the inventive method described herein, and any and all plant parts and propagules thereof. The present invention extends further to encompass the progeny derived from a primary transformed or transfected cell, tissue, organ or whole plant that has been produced by the inventive method, the only requirement being that said progeny exhibits the same genotypic and/or phenotypic characteristic(s) as that (those) characteristic(s) that has (have) been produced in the parent by the performance of the inventive method. The invention also extents to harvestable parts of a plant such as but not limited to seeds, leaves, fruits, stem cultures, rhizomes and bulbs

In a preferred embodiment of the invention the cell cycle progression rate is significantly modified by ectopic expression, preferably constitutive expression of a cyclin D protein or a homologue or derivative thereof as defined in the current invention. As said cyclins D are indispensable for full activation of Cdc2-type or Cdc2-related protein kinases and as said cyclins D interact with Cdc2-type or Cdc2-related protein kinases required for at least G1-S transition and G2-M progresion, it is easily conceivable that elevated levels of said cyclin D result in a significant acceleration of the cell cycle progression. Alternatively, but not mutually exclusive, overexpression of said cyclin D might titrate out CKI inhibitor molecules and hence contribute to full activation of Cdc2-type or Cdc2-related protein kinases. It is also clear to the skilled artisan that ectopic expression, preferably constitutive expression of said cyclin D can promote and extend cell division activity in cells that normally become quiescent during the course of development and/or as a consequence of adverse growth conditions and/or as a consequence of stress conditions Ectopic overexpression, preferably constitutive expression of said cyclin D can thus be expected to increase the frequency of the formation of lateral organs including leaves (resulting in increased bushiness), flowers (resulting in increased numbers of seeds or seed pods) and roots (resulting in increased numbers of lateral roots). The timing of lateral organ formation can also be altered, e.g. resulting in earlier flowering. Another expected effect of delaying cells to become quiescent is the delayed occurrence of senescence. Ectopic expression, preferably constitutive expression of said cyclin D is furthermore expected to enhance growth under conditions of e.g. salt or drought stress.

It will be clear to the skilled artisan that said effects obtained by ectopic expression of a cyclin D protein or a homologue or derivative thereof according to the present invention may as well be obtained by down-regulation of expression of the barrel medic CKIMt or a functional homologue thereof.

When ectopic expression of said cyclin D is occurring at the whole plant level, an overall growth enhancing effect is to be expected, i.e. recombinant plants will grow faster and/or will reach a larger size. This is particularly useful to increase yield of e.g. fodder plants, forage plants, leguminous plants and wood-producing plants. When ectopic expression of said cyclin D is confined to single cells, tissues or organs of a plant, the growth enhancing effect will be confined to said single cells, tissues or organs of said plant. Particularly useful are restrictions of ectopic overexpression of said cyclin D to tissues or organs including seeds, fruits, tubers, roots, shoots, stems and nodules to increase yield and/or size of said tissues or organs.

Thus, in the current invention is included a most preferred embodiment comprising ectopic expression of a cyclin D protein or a homologue or derivative thereof as defined in the current invention in a plant cell and/or tissue and/or organ to obtain enhanced growth and/or delayed senescence of said plant cell and/or tissue and/or organ or to obtain enhanced formation of lateral organs from said plant tissue and/or organ.

In another preferred embodiment ectopic expression, preferably constitutive expression of a cyclin D protein or a homologue or derivative thereof as defined in the current invention in a whole plant results in enhanced growth and/or in increased frequency of lateral organ formation and/or delayed senescence of said plant.

Futher embodied in the invention is significant modification of the cell cycle progression rate by enhancing cell cycle phase-specific expression of a cyclin D protein or a homologue or derivative thereof as defined in the current invention. Preferred is the enhanced expression of said cyclin D during M-phase. The resulting effect will be the ability to fully activate Cdc2-type or Cdc2-related protein kinase specifically active during M-phase such as B-type PPTLLRE Cdc2 and B-type PPTALRE Cdc2. Alternatively, but not mutually exclusive, overexpression of said cyclin D might titrate out CKI inhibitor molecules and hence contribute to full activation of Cdc2-type or Cdc2-related protein kinases. Under such conditions M-phase can be completed faster which will recapitulate those obtained with constitutive expression of said cyclin D as described supra.

Also considered as an embodiment of the invention is the ectopic expression, including constitutive expression, of a cyclin D protein or a homologue or derivative thereof as defined in the current invention together with modulating expression levels of a target protein of said cyclin D. Preferred targets of said cyclin D include Cdc2-type or Cdc2-related kinases, more specifically B-type PPTTLRE Cdc2, B-type PPTALRE Cdc2 and A-type PSTAIRE Cdc2. Other targets include the barrel medic CKIMt or functional homologues thereof. Co-overexpression of said cyclin D protein and said Cdc2-type or Cdc2-related kinase protein is expected to enhance the effects obtainable with ectopic expression of said cyclin D alone. Alternatively, ectopic expression of said cyclin D protein combined with downregulation of expression of the barrel medic CKIMt or a functional homologue thereof is also expected to enhance the effects obtainable with ectopic expression of said cyclin D alone.

Expression of said preferred targets of cyclin D is preferentially modulated during the phases of natural expression of said targets and can be achieved e.g. by gene duplication as a result of the introduction of a recombinant gene copy.

Yet another preferred embodiment of the invention comprises significant modification of the cell cycle progression rate by downregulation of expression of a cyclin D protein or a homologue or derivative thereof as defined in the current invention. As said cyclins D are indispensable for full activation of Cdc2-type or Cdc2-related protein kinases and as said cyclins D interact with Cdc2-type or Cdc2-related protein kinases required for at least G1-S transition and G2-M progresion, it is easily conceivable that decreased levels of said cyclin D result in a significant inhibition of the cell cycle progression. Thus, effects opposite to those obtainable as described for ectopic expression of said cyclin D can be expected. Said opposite effects have useful applications as described infra. As will be clear to the skilled artisan, said opposite effects can also be obtained via ectopic expression of the barrel medic CKIMt or a functional homologue thereof, thus decreasing the availability of said cyclin D to interact with Cdc2-type or Cdc2-related kinases.

Downregulation of expression of said cyclin D or upregulation of expression of said CKI at the whole plant level can e.g. create dwarfism. Downregulation of expression of said cyclin D or upregulation of expression of said CKI in specific cells, tissues or organs can find applications such as the inhibition of side shoot formation in crops such as tomato.

Downregulation of expression of said cyclin D or upregulation of expression of said CKI as a result of pathogen infection might confer enhanced resistance to pathogens causing neoplastic plant growth such as plant pathogenic bacteria including. Agrobacterium tumefaciens, plant pathogenic fungi including Plasmodiophora brassicae, Crinipellis pemiciosa, Pucciniastrum geoppertianum, Taphrina wiesneri, Ustilaga maydis, Exobasidium vaccinii, E. camelliae, Entorrhiza casparyana and Apiosporina morbosum.

Yet another preferred embodiment of the invention comprises significant modification of the cell cycle progression rate by cell cycle phase-specific downregulatlon of expression of a cyclin D protein or a homologue or derivative thereof as defined in the current invention or upregulation of expression of the barrel medic CKIMt or a functional homologue thereof. Preferred is the downregulation of expression of said cyclin D or upregulation of said CKI during M-phase. The resulting effect will be the inability to fully activate Cdc2-type or Cdc2-related protein kinase specifically active during M-phase such as B-type PPTLLRE Cdc2 and B-type PPTALRE Cdc2. Under such conditions M-phase can not be completed and cells are stimulated to undergo endoreduplication cycles, i.e. passage through consecutive cell cycles including DNA replication but without intervening cytokinesis. Cells undergoing endoreduplication thus become polyploid. Downregulation of expression of said cyclin D has potential applications including increasing seed yield and/or seed size. Those skilled in the art will be aware that grain yield in crop plants is largely a function of the amount of starch produced in the endosperm of the seed. The amount of protein produced in the endosperm is also a contributing factor to grain yield. In contrast, the embryo and aleurone layers contribute little in terms of the total weight of the mature grain. By virtue of being linked to cell expansion and metabolic activity, endoreduplication is generally considered as an important factor for increasing yield (Traas et al 1998). As grain endosperm development initially includes extensive endoreduplication (Olsen et al 1999), enhancing, promoting or stimulating this process is likely to result in increased grain yield. Enhancing, promoting or stimulating cell division during seed development as described supra is an alternative way to increase grain yield.

Methods for the production of silica or SiO₂ from peels or husks of larger rice seeds obtained according to the described inventive methods are also subject of the current invention.

Further embodied by the invention is the ectopic expression of a cyclin D protein or a homologue or derivative thereof as defined in the current invention as a result of pathogen infection which might confer enhanced resistance to pathogens relying on endoreduplication events in the infected host cells to survive. The ectopic expression of said cyclin D induced by infection is expected to promote completion of the cell cycle including cytokinesis and thus to inhibit endoreduplication events. Pathogens relying on host cell endoreduplication, e.g. to establish a feeding structure, include nematodes such as Heterodera species and Meloidogyne species.

The present invention is applicable to any plant, in particular a monocotyledonous plants and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba fadnosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incarnata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesil, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp. Vitis vinifera, Watsonia pyramidata, Zantedeschla aethiopica, Zea mays, amaranth, artichoke, asparagus, broccoli, brussel sprout, cabbage, canola, carrot, cauliflower, celery, collard greens, flax, kale, lentil, oilseed rape, okra, onion, potato, rice, soybean, straw, sugarbeet, sugar cane, sunflower, tomato, squash, and tea, amongst others, or the seeds of any plant specifically named above or a bissue, cell or organ culture of any of the above species.

The present invention further relates to a method for identifying and obtaining agonists of a cyclin D protein or a homologue or derivative thereof as defined in the current invention and/or of the interaction of said cyclin D with Cdc2-type or Cdc2-related kinases, e.g. the barrel medic CKIMt as exemplified in the current invention or a functional homologue thereof.

In one embodiment, said agonists target domains of said cyclin D including domains important for interaction with Cdc2-type or Cdc2-related kinases and other domains of said cyclin D expected to be required for interaction of said cyclin D with other targets of said cyclin D or of said cyclin D complexed with a Cdc2-type or Cdc2-related kinase. Preferably said agonists are chemical compounds which can find uses as e.g. plant growth regulators or herbicides. Methods to identify such compounds include addition of said compounds to a yeast two-hybrid system wherein said cyclin D and and interacting Cdc2-type or Cdc2-related kinase are expressed. Another such method comprises real-time measurement of interaction of any of said compounds with said cyclin D or with said cyclin D complexed with a Cdc2-type or Cdc2-related kinase using the BIACore apparatus (Pharmacia).

In another embodiment, said agonists are protein partners capable of interacting with said cyclin D. As described supra, methods to identify such proteins include for instance a two-hybrid system and immunoprecipitation.

Therefore, the present invention relates to a method for identifying and obtaining proteins interacting with plant type D-cyclin comprising a two-hybrid screening assay wherein a polypeptide of according to the invention as a bait and a cDNA library as prey are used.

Furthermore, the present invention relates to a method for modulating the interaction between and/or the activity of complexes comprising type D-cyclin and Cdc2-type or Cdc2-related kinases and/or other D-cyclin interacting protein partners obtainable by a method as defined above comprising the use of a polypeptide of the invention. Said other D-cyclin interacting protein partner can e.g. be the barrel medic cyclin dependent kinase inhibitor or a functional homologue thereof. Alternatively, the barrel medic cyclin dependent kinase inhibitor or a functional homologue thereof can be used to modulate the interaction between and/or the activity of complexes comprising type D-cyclin and Cdc2-type or Cdc2-related kinases.

In a preferred embodiment, the method for identifying and obtaining compounds interacting with plant type D-cyclin comprises the following steps:
a) providing a yeast two-hybrid system wherein a polypeptide of the invention and a Cdc2-type or Cdc2-related kinase or other D-cyclin interacting protein partners obtainable by the above described method are expressed,
b) interacting said compound with the complex formed by the expressed polypeptides as defined in a).

In said method, the other D-cyclin interacting protein partner can be the barrel medic cyclin dependent kinase inhibitor or a functional homologue thereof.

In another embodiment, the present invention relates to a method for identifying compounds or mixtures of compounds which specifically bind to a polypeptide of the invention as defined earlier, comprising:
a) combining a polypeptide of the invention with said compound or mixtures of compounds under conditions suitable to allow complex formation, and,
b) detecting complex formation, wherein the presence of a complex identifies a molecule which specifically binds said polypeptide.

As such, the invention also relates to the use of a molecule identified by means of a method as described above as a plant growth regulator or herbicide.

According to another embodiment, the invention also relates to a method for production of a plant growth regulator or herbicide composition comprising the steps of the methods described above and formulating the compounds obtained from said steps in a suitable form for the application in agriculture or plant cell or tissue culture.

The invention also extends to the use of any of the nucleic acid molecules, the vectors, the host cells, the polypeptides and the antibodies of the invention for modifying cell fate, for modifying plant development and/or for modifying plant morphology and/or for modifying plant physiology and/or for modifying plant biochemistry.

The invention also extends to a diagnostic composition comprising at least a nucleic acid molecule, a vector, a host cell, a polypeptide or an antibody of the invention.

Definitions and Elaborations to the Embodiments

The terms “protein(s)”, “peptide(s)” or “oligopeptide(s)”, when used herein refer to amino acids in a polymeric form of any length. Said terms also include known amino acid modifications such as disulphide bond formation, cysteinylation, oxidation, glutathionylation, methylation, acetylation, farnesylation, biotinylation, stearoylation, formylation, lipoic acid addition, phosphorylation, sulphation, ubiquitination, myrlstoylation, palmitoylation, geranylgeranylation, cyclization (e.g. pyroglutamic acid formation), oxidation, deamidation, dehydration, glycosylation (e.g. pentoses, hexosamines, N-acetylhexosamines, deoxyhexoses, hexoses, sialic acid etc.) and acylation as well as non-naturally occurring amino acid residues, L-amino acid residues and D-amino acid residues.

“Homologues” of a protein of the invention are those peptides, oligopeptides, polypeptides, proteins and enzymes which contain amino acid substitutions, deletions and/or additions relative to the said protein with respect to which they are a homologue, without altering one or more of its functional properties, in particular without reducing the activity of the resulting. For example, a homologue of said protein will consist of a bioactive amino acid sequence variant of said protein. To produce such homologues, amino acids present in the said protein can be replaced by other amino acids having similar properties, for example hydrophobicity, hydrophilicity, hydrophobic moment, antigenicity, propensity to form or break α-helical structures or β-sheet structures; and so on. An overview of physical and chemical properties of amino acids is given in Table 5.

TABLE 5
Properties of naturally occurring amino acids.

| Charge properties/ | | |
|---|---|---|
| hydrophobicity | Side group | Amino Acid |
| nonpolar | Aliphatic | ala, ile, leu, |
| hydrophobic | aliphatic, S-containing | val |
| | aromatic | met |
| | imino | phe, trp |
| | | pro |
| polar uncharged | Aliphatic | gly |
| | amide | asn, gln |
| | aromatic | tyr |
| | hydroxyl | ser, thr |
| | sulfhydryl | cys |
| positively charged | Basic | arg, his, lys |
| negatively charged | Acidic | asp, gly |

Substitutional variants of a protein of the invention are those in which at least one residue in said protein amino acid sequence has been removed and a different residue inserted in its place. Amino acid substitutions are typically of single residues, but may be clustered depending upon functional constraints placed upon the polypeptide; insertions will usually be of the order of about 1-10 amino acid residues and deletions will range from about 1-20 residues. Preferably, amino acid substitutions will comprise conservative amino acid substitutions, such as those described supra.

Insertional amino acid sequence variants of a protein of the invention are those in which one or more amino acid residues are introduced into a predetermined site in said protein. Insertions can comprise amino-terminal and/or carboxy-terminal fusions as well as intra-sequence insertions of single or multiple amino acids. Generally, insertions within the amino acid sequence will be smaller than amino or carboxyl terminal fusions, of the order of about 1 to 10 residues. Examples of amino- or carboxy-terminal fusion proteins or peptides include the binding domain or activation domain of a transcriptional activator as used in a two-hybrid system, phage coat proteins, (histidine)₆-tag, glutathione S-transferase, protein A, maltose-binding protein, dihydrofolate reductase, Tag•100 epitope (EETARFQPGYRS; SEQ ID NO 22), c-myc epitope (EQKLISEEDL; SEQ ID NO 23), FLAG®-epitope (DYKDDDK; SEQ ID NO 24), lacZ, CMP (calmodulin-binding peptide), HA epitope (YPYDVPDYA; SEQ ID NO 25), protein C epitope (EDQVDPRLIDGK; SEQ ID NO 26) and VSV epitope (YTDIEMNRLGK; SEQ ID NO 27).

Deletional variants of a protein of the invention are characterised by the removal of one or more amino acids from the amino acid sequence of said protein.

Amino acid variants of a protein of the invention may readily be made using peptide synthetic techniques well known in the art, such as solid phase peptide synthesis and the like, or by recombinant DNA manipulations. The manipulation of DNA sequences to produce variant proteins which manifest as substitutional, insertional or deletional variants are well known in the art For example, techniques for making substitution mutations at predetermined sites in DNA having known sequence are well known to those skilled in the art, such as by M13 mutagenesis, T7-Gen in vitro mutagenesis kit (USB, Cleveland, Ohio), QuickChange Site Directed mutagenesis kit (Stratagene, San Diego, Calif.), PCR-mediated site-directed mutagenesis or other site-directed mutagenesis protocols.

“Derivatives” of a protein of the invention are those peptides, oligopeptides, polypepudes, proteins and enzymes which comprise at least about five contiguous amino acid residues of said polypeptide but which retain the biological activity of said protein. A “derivative” may further comprise additional naturally-occurring, altered glycosylated, acylated or non-naturally occurring amino acid residues compared to the amino acid sequence of a naturally-occurring form of said polypeptide. Alternatively or in addition, a derivative may comprise one or more non-amino acid substituents compared to the amino acid sequence of a naturally-occurring form of said polypeptide, for example a reporter molecule or other ligand, covalently or non-covalently bound to the amino acid sequence such as, for example, a reporter molecule which is bound thereto to facilitate its detection.

With “immunologically active” is meant that a molecule or specific fragments thereof such as epitopes or haptens are recognized by, i.e. bind to antibodies.

In the context of the current invention are embodied homologues, derivatives and/or immunologically active fragments of an inventive cyclin D as defined supra. Particularly preferred homologues, derivatives and/or immunologically active fragments of a cyclin D protein which are contemplated for use in the current invention are derived from plants, more specifically from barrel medic, even more specifically said cyclin D is the barrel medic MtCycDm, or are capable of being expressed therein. The present invention clearly contemplates the use of functional homologues or derivatives and/or immunologically active fragments of the MtCycDm protein and is not to be limited in application to the use of a nucleotide sequence encoding said MtCycDm protein.

“Antibodies” include monoclonal, polyclonal, synthetic or heavy chain camel antibodies as well as fragments of antibodies such as Fab, Fv or scFv fragments. Monoclonal antibodies can be prepared by the techniques as described in e.g. Liddle and Cryer (1991) which comprise the fusion of mouse myeloma cells to spleen cells derived from immunized animals. Furthermore, antibodies or fragments thereof to a molecule or fragments thereof can be obtained by using methods as described in e.g. Harlow and Lane (1988). In the case of antibodies directed against small peptides such as fragments of a protein of the invention, said peptides are generally coupled to a carrier protein before immunization of animals. Such protein carriers include keyhole limpet hemocyanin (KLH), bovine serum albumin (BSA), ovalbumin and Tetanus toxoid. The carrier protein enhances the immune response of the animal and provides epitopes for T-cell receptor binding sites. The term “antibodies” furthermore includes derivatives thereof such as labelled antibodies. Antibody labels include alkaline phosphatase, PKH2, PKH26, PKH67, fluorescein (FITC), Hoechst 33258, R-phycoerythrin (PE), rhodamine (TRITC), Quantum Red, Texas Red, Cy3, biotin, agarose, peroxidase and gold spheres. Tools in molecular biology relying on antibodies against a protein include protein gel blot analysis, screening of expression libraries allowing gene identification, protein quantitative methods including ELISA and RIA, immunoaffinity purification of proteins, immunoprecipitation of proteins (see e.g. Example 5) and immunolocalization (see e.g. Example 6). Other uses of antibodies and especially of peptide antibodies include the study of proteolytic processing (Loffier et al. 1994, Woulfe et al. 1994), determination of protein active sites (Lerner 1982), the study of precursor and post-translational processing (Baron and Baltimore 1982, Lerner et al. 1981, Semler et al. 1982), identification of protein domains involved in protein-protein interactions (Murakami et al. 1992) and the study of exon usage in gene expression (Tamura et al. 1991).

Embodied in the current invention are antibodies specifically recognizing a cyclin D or homologue, derivative or fragment thereof as defined supra. Preferably said cyclin D is a plant cyclin D, more specifically the barrel medic MtCycDm.

The terms “gene(s)”, “polynucleotide(s)”, “nucleic acid(s)”, “nucleic acid sequence(s)”, “nucleotide sequence(s)”, or “nucleic acid molecule(s)”, when used herein refer to nucleotides, either ribonucleotides or deoxyribonucleotides or a combination of both, in a polymeric form of any length. Said terms furthermore include double-stranded and single-stranded DNA and RNA. Said terms also include known nucleotide modifications such as methylation, cyclization and ‘caps’ and substitution of one or more of the naturally occurring nucleotides with an analog such as inosine. Modifications of nucleotides include the addition of acridine, amine, biotin, cascade blue, cholesterol, Cy3®, Cy5®, Cy5.5® Dabcyl, digoxigenin, dinitrophenyl, Edans, 6-FAM, fluorescein, 3′-glyceryl, HEX, IRD700, IRD-800, JOE, phosphate psoralen, rhodamine, ROX, thiol (SH), spacers, TAMRA, TET, AMCA-S®, SE, BODIPY®, Marina Blue®, Pacific Blue®, Oregon Green®, Rhodamine Green®, Rhodamine Red®, Rhodol Greene® and Texas Red®. Polynucleotide backbone modifications include methylphosphonate, 2′-OMe-methylphosphonate RNA, phosphorothiorate, RNA, 2′-OMeRNA. Base modifications include 2-amino-dA, 2-aminopurine, 3′-(ddA), 3′dA(cordycepin), 7-deaza-dA, 8-Br-dA, 8-oxo-dA, N⁶-Me-dA, abasic site (dSpacer), biotin dT, 2′-OMe-5Me-C, 2′-OMe-propynyl-C, 3′-(5-Me-dC), 3′-(ddC), 5-Br-dC, 5-I-dC, 5-Me-dC, 5-F-dC, carboxy-dT, convertible dA, convertible dC, convertible dG, convertible dT, convertible dU, 7-deaza-dG, 8-Br-dG, 8-oxo-dG, O⁶-Me-dG, S6-DNP-dG, 4methyl-indole, 5-nitroindole, 2′-OMe-inosine, 2′-dI, 0⁶-phenyl-dI, 4methyl-indole, 2′-deoxynebularine, 5-nitroindole, 2-aminopurine, dP(purine analogue), dK(pyrimidine analogue), 3-nitropyrrole, 2-thio-dT, 4-thio-dT, biotin-dT, carboxy-dT, O⁴-Me-dT, O⁴-triazol dT, 2′-OMe-propynyl-U, 5-Br-dU, 2′-dU, 5-F-dU, 5-I-dU, O⁴-triazol dU. Said terms also encompass peptide nucleic acids (PNAs), a DNA analogue in which the backbone is a pseudopeptide consisting of N-(2-aminoethyl)-glycine units rather than a sugar. PNAs mimic the behaviour of DNA and bind complementary nucleic acid strands. The neutral backbone of PNA results in stronger binding and greater specificity than normally achieved. In addition, the unique chemical, physical and biological properties of PNA have been exploited to produce powerful biomolecular tools, antisense and antigene agents, molecular probes and biosensors.

The present invention also advantageously provides nucleic acid sequences of at least approximately 15 contiguous nucleotides of a nucleic acid according to the invention and preferably from 15 to 50 nucleotides. These sequences may, advantageously be used as probes to specifically hybridise to sequences of the invention as defined above or primers to initiate specific amplification or replication of sequences of the invention as defined above, or the like. Such nucleic acid sequences may be produced according to techniques well known in the art, such as by recombinant or synthetic means. They may also be used in diagnostic kits or the like for detecting the presence of a nucleic acid according to the invention. These tests generally comprise contacting the probe with the sample under hybridising conditions and detecting the presence of any duplex or triplex formation between the probe and any nucleic acid in the sample.

Advantageously, the nucleic acid sequences, according to the invention may be produced using such recombinant or synthetic means, such as for example using PCR cloning mechanisms which generally involve making a pair of primers, which may be from approximately 15 to 50 nucleotides to a region of the gene which is desired to be cloned, bringing the primers into contact with mRNA, cDNA or genomic DNA from a human cell, performing a polymerase chain reaction under conditions which bring about amplification of the desired region, isolated the amplified region or fragment and recovering the amplified DNA. Generally, such techniques as defined herein are well known in the art, such as described in Sambrook et al. (Molecular Cloning: a Laboratory Manual, 1989).

Preferred lengths of nucleic acids specifically hybridising or specifically amplifying the nucleic acids of the invention are 20, 25, 30, 35, 40 or 45 nucleotides in length. A “coding sequence” or “open reading frame” or “ORF” is defined as a nucleotide sequence that can be transcribed into mRNA and/or translated into a polypeptide when placed under the control of appropriate regulatory sequences, i.e. when said coding sequence or ORF is present in an expressible format. Said coding sequence of ORF is bounded by a 5′ translation start codon and a 3′ translation stop codon. A coding sequence or ORF can include, but is not limited to RNA, mRNA, cDNA, recombinant nucleotide sequences, synthetically manufactured nucleotide sequences or genomic DNA. Said coding sequence or ORF can be interrupted by intervening nucleic acid sequences.

Genes and coding sequences essentially encoding the same protein but isolated from different sources can consist of substantially divergent nucleic acid sequences. Reciprocally, substantially divergent nucleic acid sequences can be designed to effect expression of essentially the same protein. Said nucleic acid sequences are the result of e.g. the existence of different alleles of a given gene, of the degeneracy of the genetic code or of differences in codon usage. Thus, as indicated in Table 6, amino acids such as methionine and tryptophan are encoded by a single codon whereas other amino acids such as arginine, leucine and serine can each be translated from up to six different codons. Differences in preferred codon usage are illustrated below for Agrobacterium tumefaciens (a bacterium), A. thaliana, M. sativa (two dicotyledonous plants) and Oryza sativa (a monocotyledonous plant). These examples were extracted from http://www.kazusa.or.jp/codon. For example, the codon GGC (for glycine) is the most frequently used codon in A. tumefaciens (36.2‰), is the second most frequently used codon in O. sativa but is used at much lower frequencies in A. thaliana and M. sativa (9‰ and 8.4‰, respectively). Of the four possible codons encoding glycine (see Table 6), said GGC codon is most preferably used in A. tumefaciens and O. sativa. However, in A. thaliana this is the GGA (and GGU) codon whereas in M. sativa this is the GGU (and GGA) codon.

“Hybridization” is the process wherein substantially homologous complementary nucleotide sequences anneal to each other. The hybridization process can occur entirely in solution, i.e. both complementary nucleic adds are in solution. Tools in molecular biology relying on such a process include PCR, subtractive hybridization and DNA sequence determination. The hybridization process can also occur with one of the complementary nucleic acids immobilized to a matrix such as magnetic beads, Sepharose beads or any other resin. Tools in molecular biology relying on such a process include the isolation of poly (A+)mRNA. The hybridization process can furthermore occur with one of the complementary nucleic acids immobilized to a solid support such as a nitrocellulose or nylon membrane or immobilized by e.g. photolitography to e.g. a silicious glass support (the latter known as nucleic acid arrays or microarrays or as nucleic acid chips). Tools in molecular biology relying on such a process include RNA and DNA gel blot analysis, colony hybridization, plaque hybridization and microarray hybridization. In order to allow hybridization to occur, the nucleic acid molecules are generally thermally or chemically (e.g. by NaOH) denatured to melt a double strand into two single strands and/or to remove hairpins or other secondary structures from single stranded nucleic acids. The stringency of hybridization is influenced by conditions such as temperature, salt concentration and hybridization buffer composition. High stringency conditions for hybridization include high temperature and/or low salt concentration (salts include NaCl and Na3citrate) and/or the inclusion of formamide in the hybridization buffer and/or lowering the concentration of compounds such as SDS (detergent) in the hybridization buffer and/or exclusion of compounds such as dextran sulfate or polyethylene glycol (promoting molecular crowding) from the hybridization buffer. Conventional hybridization conditions are described in e.g. Sambrook et al. (1989) but the skilled craftsman will appreciate that numerous different hybridization conditions can be designed in function of the known or the expected homology and/or length of the nucleic acid sequence. Generally, for hybridizations with DNA probes without formamide, a temperature of 68° C., and for hybridization with formamide, 50% (v/v), a temperature of 42° C. is recommended. For hybridizations with oligonucleotides, the optimal conditions (formamide concentration and/or temperature) depend on the length of the probe and must be determined individually. When using RNA probes with formamide (50% v/v) it is recommend to use a hybridization temperature of 68° C. for detection of target RNA and of 50° C. for detection of target DNA. Alternatively, a high SDS hybridization solution can be utilized (Church and Gilbert 1984).

Sufficiently low stringency hybridization conditions are particularly preferred to isolate nucleic acids heterologous to the DNA sequences of the invention defined supra. Elements contributing to said heterology include allelism, degeneration of the genetic code and differences in preferred codon usage as discussed supra.

With “specifically hybridising” is meant herein that hybridisation conditions are sufficiently stringent to obtain a high signal-to-noise ratio; optimally the noise is reduced to near zero. Specifically hybridising sequences typically have about at least 80% sequence identity, preferably 90% sequence identity, and most preferably 99 or 100% sequence identity with each other.

Percentage identity between two or more nucleic acid sequences can be calculated for instance by using the GAP program of the GCG program package (Wisconsin Package, Version 8, September 1994, Genetics Computer Group, 575 Science Drive, Madison, Wis., USA 53711). Default parameter settings can be used. GAP considers all possible alignments and gap positions between two sequences and creates a global alignment (thus not a local alignment as obtained with e.g. BLAST algorithms) that maximizes the number of matched residues and minimizes the number and size of gaps. A scoring matrix is used to assign values for symbol matches. In addition, a gap creation penalty and a gap extension penalty are required to limit the insertion of gaps into the alignment. GAP makes an alignment to find the maximum similarity between two sequences by the method of Needleman and Wunsch (1970). Clearly, the current invention embodies the use of the inventive DNA sequences encoding a cyclin D, homologue, derivative and/or immunologically fragment thereof as defined higher in any method of hybridization. The current invention furthermore also related to DNA sequences hybridizing to said inventive DNA sequences. Preferably said cyclin D is a plant cyclin D, more specifically the barrel medic MtCycDm.

DNA sequences as defined in the current invention can be interrupted by intervening sequences. With “intervening sequences” is meant any nucleic acid sequence which disrupts a coding sequence comprising said inventive DNA sequence or which disrupts the expressible format of a DNA sequence comprising said inventive DNA sequence. Removal of the intervening sequence restores said coding sequence or said expressible format. Examples of intervening sequences include introns and mobilizable DNA sequences such as transposons. With “mobilizable DNA sequence” is meant any DNA sequence that can be mobilized as the result of a recombination event.

TABLE 6
Degeneracy of the genetic code.

| | Three- | One- | | | | | | |
|---|---|---|---|---|---|---|---|---|
| | letter | letter | | | | | | |
| Amino Acid | code | code | | | | | | |
| | | | Possible codons | | | | | |
| Alanine | Ala | A | GCA | GCC | GCG | GCU | | |
| Arginine | Arg | R | AGA | AGG | CGA | CGC | CGG | CGU |
| Asparagine | Asn | N | AAC | AAU | | | | |
| Aspartic | Asp | D | GAC | GAU | | | | |
| Acid | | | | | | | | |
| Cysteine | Cys | C | UGC | UGU | | | | |
| Glutamic | Glu | E | GAA | GAG | | | | |
| Acid | | | | | | | | |
| Glutamine | Gln | Q | CAA | CAG | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Leucine | Leu | L | UUA | UUG | CUA | CUC | CUG | CUU |
| Lysine | Lys | K | AAA | AAG | | | | |
| Methionine | Met | M | AUG | | | | | |
| Phenyl- | Phe | F | UUC | UUU | | | | |
| alanine | | | | | | | | |
| Proline | Pro | P | CCA | CCC | CCG | CCU | | |
| Serine | Ser | S | AGC | AGU | UCA | UCC | UCG | UCU |
| Threonine | Thr | T | ACA | ACC | ACG | ACU | | |
| Tryptophan | Trp | W | UGG | | | | | |
| Tyrosine | Tyr | Y | UAC | UAU | | | | |
| Valine | Val | V | GUA | GUC | GUG | GUU | | |

| Possible “STOP” codons | | |
|---|---|---|
| UAA | UAG | UGA |

To effect expression of a protein in a cell, tissue or organ, preferably of plant origin, either the protein may be introduced directly to said cell, such as by microinjection or ballistic means or alternatively, an isolated nucleic acid molecule encoding said protein may be introduced into said cell, tissue or organ in an expressible format.

Preferably, the DNA sequence of the invention comprises a coding sequence or open reading frame (ORF) encoding a cyclin D protein or a homologue or derivative thereof or an immunologically active thereof as defined supra. The preferred protein of the invention comprises the amino acid sequence of said cyclin D. Preferably said cyclin D is a plant cyclin D and more specifically the barrel medic MtCycDm.

With “vector” or “vector sequence” is meant a DNA sequence which can be introduced in an organism by transformation and can be stably maintained in said organism. Vector maintenance is possible in e.g. cultures of Eschedchia coli, A. tumefaciens, Saccharomyces cerevisiae or Schizosaccharomyces pombe. Other vectors such as phagemids and cosmid vectors can be maintained and multiplied in bacteria and/or viruses. Vector sequences generally comprise a set of unique sites recognized by restriction enzymes, the multiple cloning site (MCS), wherein one or more non-vector sequence(s) can be inserted.

With “non-vector sequence” is accordingly meant a DNA sequence which is integrated in one or more of the sites of the MCS comprised within a vector.

“Expression vectors” form a subset of vectors which, by virtue of comprising the appropriate regulatory sequences enabling the creation of an expressible format for the inserted non-vector sequence(s), thus allowing expression of the protein encoded by said non-vector sequence(s). Expression vectors are known in the art enabling protein expression in organisms including bacteria (e.g. E. coli), fungi (e.g. S. cerevisiae, S. pombe, Pichia pastoris), insect cells (e.g. baculoviral expression vectors), animal cells (e.g. COS or CHO cells) and plant cells (e.g. potato virus X-based expression vectors). The current invention clearly includes any vector or expression vector comprising a non-vector DNA sequence encoding a cyclin D, homologue, derivative and/or immunologically active fragment thereof as defined supra. Preferably said cyclin D is a plant cyclin D, more specifically barrel medic MtCycDm.

As an alternative to expression vector-mediated protein production in biological systems, chemical protein synthesis can be applied. Synthetic peptides can be manufactured in solution phase or in solid phase. Solid phase peptide synthesis (Merrifield 1963) is, however, the most common way and involves the sequential addition of amino acids to create a linear peptide chain. Solid phase peptide synthesis includes cycles consisting of three steps: (i) immobilization of the carboxy-terminal amino acid of the growing peptide chain to a solid support or resin; (ii) chain assembly, a process consisting of activation, coupling and deprotection of the amino acid to be added to the growing peptide chain; and (iii) cleavage involving removal of the completed peptide chain from the resin and removal of the protecting groups from the amino acid side chains. Common approaches in solid phase peptide synthesis include Fmoc/tBu (9-fluorenylmethyloxycarbonyl/t-butyl) and Boc (t-butyloxycarbonyl) as the amino-terminal protecting groups of amino acids. Amino acid side chain protecting groups include methyl (Me), formyl (CHO), ethyl (Et), acetyl (Ac), t-butyl (t-Bu), anisyl, benzyl (Bzl), trifluroacetyl (Tfa), N-hydroxysuccinimide (ONSu, OSu), benzoyl (Bz), 4-methylbenzyl (Meb), thioanizyl, thiocresyl, benzyloxymethyl (Bom), 4-nitrophenyl (ONp), benzyloxycarbonyl (Z), 2-nitrobenzoyl (NBz), 2-nitrophenylsulphenyl (Nps), 4-toluenesulphonyl (Tosyl,Tos), pentafluorophenyl (Pfp), diphenylmethyl (Dpm), 2-chlorobenzyloxycarbonyl (Cl-Z), 2,4,5-trichlorophenyl, 2-bromobenzyloxycarbonyl (Br-Z), tripheylmethyl (Trityl, Trt), and 2,5,7,8-pentamethyl-chroman-6-sulphonyl (Pmc). During chain assembly, Fmoc or Boc are removed resulting in an activated amino-terminus of the amino acid residue bound to the growing chain. The carboxy-terminus of the incoming amino acid is activated by conversion into a highly reactive ester, e.g. by HBTU. With current technologies (e.g. PerSeptive Biosystems 9050 synthesizer, Applied Biosystems Model 431A Peptide Synthesizer), linear peptides of up to 50 residues can be manufactured. A number of guidelines is available to produce peptides that are suitable for use in biological systems including (i) limiting the use of difficult amino acids such as cys, met, trp (easily oxidized and/or degraded during peptide synthesis) or arg; (ii) minimize hydrophobic amino acids (can impair peptide solubility); and (iii) prevent an amino-terminal glutamic acid (can cyclize to pyroglutamate).

By “expressible format” is meant that the isolated nucleic acid molecule is in a form suitable for being transcribed into mRNA and/or translated to produce a protein, either constitutively or following induction by an intracellular or extracellular signal, such as an environmental stimulus or stress (mitogens, anoxia, hypoxia, temperature, salt, light, dehydration, etc) or a chemical compound such as IPTG (isopropyl-β-D-thiogalactopyranoside) or such as an antibiotic (tetracycline, ampicillin, rifampicin, kanamycin), hormone (e.g. gibberellin, auxin, cytokinin, glucocorticoid, brassinosteroid, ethylene, abscisic acid etc), hormone analogue (iodoacetic acid (IAA), 2,4D, etc), metal (zinc, copper, iron, etc), or dexamethasone, amongst others. As will be known to those skilled in the art, expression of a functional protein may also require one or more post-translational modifications, such as glycosylation, phosphorylation, dephosphorylation, or one or more protein-protein interactions, amongst others. All such processes are included within the scope of the term “expressible format”.

Preferably, expression of a protein in a specific cell, tissue, or organ, preferably of plant origin, is effected by introducing and expressing an isolated nucleic acid molecule encoding said protein, such as a cDNA molecule, genomic gene, synthetic oligonucleotide molecule, mRNA molecule or open reading frame, to said cell, tissue or organ, wherein said nucleic acid molecule is placed operably in connection with suitable regulatory sequences including a promoter, preferably a plant-expressible promoter, and a terminator sequence.

Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. The term “promoter” also includes the transcriptional regulatory sequences of a classical prokaryotic gene, in which case it may include a −35 box sequence and/or a −10 box transcriptional regulatory sequences.

The term “promoter” is also used to describe a synthetic or fusion molecule, or derivative which confers, activates or enhances expression of a nucleic acid molecule in a cell, tissue or organ.

Promoters may contain additional copies of one or more specific regulatory elements, to further enhance expression and/or to alter the spatial expression and/or temporal expression of a nucleic acid molecule to which it is operably connected. Such regulatory elements may be placed adjacent to a heterologous promoter sequence to drive expression of a nucleic acid molecule in response to e.g. copper, glucocorticoids, dexamethasone, tetracycline, gibberellin, cAMP, abscisic acid, auxin, wounding, ethylene, jasmonate or salicylic acid or to confer expression of a nucleic acid molecule to specific cells, tissues or organs such as meristems, leaves, roots, embryo, flowers, seeds or fruits.

In the context of the present invention, the promoter preferably is a plant-expressible promoter sequence. Promoters, however, that also function or solely function in non-plant cells such as bacteria, yeast cells, insect cells and animal cells are not excluded from the invention. By “plant-expressible” is meant that the promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ.

Regulatable promoters as part of a binary viral plant expression system are also known to the skilled artisan (Yadav 1999—WO9922003; Yadav 2000—WO0017365). The terms “plant-operable” and “operable in a plant” when used herein, in respect of a promoter sequence, shall be taken to be equivalent to a plant-expressible promoter sequence.

In the present context, a “regulatable promoter sequence” is a promoter that is capable of conferring expression on a structural gene in a particular cell, tissue, or organ or group of cells, tissues or organs of a plant, optionally under specific conditions, however does generally not confer expression throughout the plant under all conditions. Accordingly, a regulatable promoter sequence may be a promoter sequence that confers expression on a gene to which it is operably connected in a particular location within the plant or alternatively, throughout the plant under a specific set of conditions, such as following induction of gene expression by a chemical compound or other elicitor.

Preferably, the regulatable promoter used in the performance of the present invention confers expression in a specific location within the plant, either constitutively or following induction, however not in the whole plant under any circumstances. Included within the scope of such promoters are cell-specific promoter sequences, tissue-specific promoter sequences, organ-specific promoter sequences, cell cycle specific gene promoter sequences, inducible promoter sequences and constitutive promoter sequences that have been modified to confer expression in a particular part of the plant at any one time, such as by integration of said constitutive promoter within a transposable genetic element (Ac, Ds, Spm, En, or other transposon).

The term “cell-specific” shall be taken to indicate that expression is predominantly in a particular cell or cell-type, preferably of plant origin, albeit not necessarily exclusively in said cell or cell-type.

Similarly,. the term “tissue-specific” shall be taken to indicate that expression is predominantly in a particular tissue or tissue-type, preferably of plant origin, albeit not necessarily exclusively in said tissue or tissue-type.

Similarly, the term “organ-specific” shall be taken to indicate that expression is predominantly in a particular organ, preferably of plant origin, albeit not necessarily exclusively in said organ.

Similarly, the term “cell cycle specific” shall be taken to indicate that expression is predominantly cyclic and occurring in one or more, not necessarily consecutive phases of the cell cycle albeit not necessarily exclusively in cyclins cells, preferably of plant origin.

Those skilled in the art will be aware that an “inducible promoter” is a promoter the transcriptional activity of which is increased or induced in response to a developmental, chemical, environmental, or physical stimulus. Similarly, the skilled craftsman will understand that a “constitutive promoter” is a promoter that is transcriptionally active throughout most, but not necessarily all parts of an organism, preferably a plant, during most, but not neccessarily all phases of its growth and development.

Those skilled in the art will readily be capable of selecting appropriate promoter sequences for use in regulating appropriate expression of the cyclin protein from publicly-available or readily-available sources, without undue experimentation.

Placing a nucleic acid molecule under the regulatory control of a promoter sequence, or in operable connection with a promoter sequence, means positioning said nucleic acid molecule such that expression is controlled by the promoter sequence. A promoter is usually, but not necessarily, positioned upstream, or at the 5′-end, and within 2 kb of the start site of transcription, of the nucleic acid molecule which it regulates. In the construction of heterologous promoter/structural gene combinations it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation in this distance can also occur.

Examples of promoters suitable for use in gene constructs of the present invention include those listed in Table 7, amongst others. The promoters listed in Table 7 are provided for the purposes of exemplification only and the present invention is not to be limited by the list provided therein. Those skilled in the art will readily be in a position to provide additional promoters that are useful in performing the present invention.

In the case of constitutive promoters or promoters that induce expression throughout the entire plant, it is preferred that such sequences are modified by the addition of nucleotide sequences derived from one or more of the tissue-specific promoters listed in Table 7, or alternatively, nucleotide sequences derived from one or more of the above-mentioned tissue-specific inducible promoters, to confer tissue-specificity thereon. For example, the CaMV 35S promoter may be modified by the addition of maize Adh1 promoter sequence, to confer anaerobically-regulated root-specific expression thereon, as described previously (Ellis et al., 1987). Another example describes conferring root specific or root abundant gene expression by fusing the CaMV35S promoter to elements of the maize glycine-rich protein GRP3 gene (Feix and Wulff 2000—WO0015662). Such modifications can be achieved by routine experimentation by those skilled in the art.

The term “terminators” refers to a DNA sequence at the end of a transcriptional unit which signals termination of transcription. Terminators are 3′-non-translated DNA sequences containing a polyadenylation signal, which facilitates the addition of polyadenylate sequences to the 3′-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the gene constructs of the present invention include the Agrobacterium tumefaciens nopaline synthase (NOS) gene terminator, the Agrobacterium tumefaciens octopine synthase (OCS) gene terminator sequence, the Cauliflower mosaic virus (CaMV) 35S gene terminator sequence, the Oryza sativa ADP-glucose pyrophosphorylase terminator sequence (t3′Bt2), the Zea mays zeln gene terminator sequence, the rbcs-1A gene terminator, and the rbcs-3A gene terminator sequences, amongst others.

TABLE 7
Exemplary plant-expressible promoters for use
in the performance of the present invention
I: CELL-SPECIFIC, TISSUE-SPECIFIC, AND ORGAN-SPECIFIC PROMOTERS

| | EXPRESSION | |
|---|---|---|
| GENE SOURCE | PATTERN | REFERENCE |
| α-amylase (Amy32b) | aleurone | Lanahan, M. B., et al., Plant Cell 4: 203-211, 1992; |
| | | Skriver, K., et al. Proc. Natl. Acad. Scl. (USA) 88: |
| | | 7266-7270, 1991 |
| cathepsin β-like gene | aleurone | Cejudo, F. J., et al. Plant Molecular Biology 20: 849-856, |
| | | 1992. |
| Agrobacterium rhizogenes rolB | cambium | Nilsson et al., Physiol. Plant 100: 456-462, 1997 |
| AtPRP4 | flowers | http://salus.medium.edu/mmg/tierney/html |
| chalcone synthase (chsA) | flowers | Van der Meer, et al., Plant Mol. Biol. 15, 95-109, |
| | | 1990. |
| LAT52 | anther | Twell et al Mol. Gen Genet. 217: 240-245 (1989) |
| apetala-3 | flowers | |
| chitinase | fruit (berries, grapes, etc) | Thomas et al. CSIRO Plant Industry, Urrbrae, South |
| | | Australia, Australia; |
| | | http://winetitles.com.au/gwrdc/csh95-1.html |
| rbcs-3A | green tissue (eg leaf) | Lam, E. et al., The Plant Cell 2: 857-866, 1990.; |
| | | Tucker et al., Plant Physiol. 113: 1303-1308, 1992. |
| leaf-specific genes | leaf | Baszczynskl, et al., Nucl. Acid Res. 16: 4732, 1988. |
| AtPRP4 | leaf | http://salus.medium.edu/mmg/tiemey/html |
| chlorella virus adenine | leaf | Mitra and Higgins, 1994, Plant Molecular Biology 26: |
| methyltransferase gene | | 85-93 |
| promoter | | |
| aldP gene promoter from rice | leaf | Kagaya et al., 1995, Molecular and General |
| | | Genetics 248: 668-674 |
| rbcs promoter from rice or | leaf | Kyozuka at al., 1993, Plant Physiology 102: 991-1000 |
| tomato | | |
| Pinus cab-6 | leaf | Yamamoto et al., Plant Cell Physiol. 35: 773-778, |
| | | 1994. |
| rubisco promoter | leaf | |
| cab (chlorophyll a/b/binding | leaf | |
| protein | | |
| SAM22 | senescent leaf | Crowell, et al., Plant Mol. Biol. 18: 459-466, 1992. |
| ltp gene (lipid transfer gene) | | Fleming, et al, Plant J. 2, 855-862. |
| R. Japonicum nif gene | nodule | U.S. Pat. No. 4,803,165 |
| B. Japonicum nifH gene | nodule | U.S. Pat. No. 5,008,194 |
| GmENOD40 | nodule | Yang, et al., The Plant J. 3: 573-585. |
| PEP carboxylase (PEPC) | nodule | Pathirana, et al., Plant Mol. Biol. 20: 437-450, 1992. |
| leghaemoglobin (Lb) | nodule | Gordon, et al., J. Exp. Bol. 44: 1453-1465, 1993. |
| Tungro bacilliform virus gene | phloem | Bhattacharyya-Pakrasl, et al, The Plant J. 4: 71-79, |
| | | 1992. |
| pollen-specific genes | Pollen; microspore | Albanl, et al., Plant Mol. Biol. 15: 605, 1990: Albanl, |
| | | et al., Plant Mol. Biol. 16: 501, 1991) |
| Zm13 | pollen | Guerrero et al Mol. Gen. Genet. 224: 161-168 (1993) |
| apg gene | microspore | Twell et al Sex. Plant Reprod. 6: 217-224 (1993) |
| maize pollen-specific gene | pollen | Hamilton, et al., Plant Mol. Biol. 18: 211-218, 1992. |
| sunflower pollen-expressed | pollen | Baltz, et al., The Plant J. 2: 713-721, 1992. |
| gene | | |
| B. napus pollen-specific gene | pollen; anther; tapetum | Arnoldo, et al., J. Cell. Biochem., Abstract No. Y101, |
| | | 204, 1992. |
| root-expressible genes | roots | Tingey, et al., EMBO J. 6: 1, 1987. |
| tobacco auxin-Inducible gene | root tip | Van der Zaal, et al., Plant Mol. Biol. 16, 983, 1991. |
| β-tubulin | root | Oppenheimer, et al., Gene 63: 87, 1988. |
| tobacco root-specific genes | root | Conkling, et al., Plant Physiol. 93: 1203, 1990. |
| B. napus G1-3b gene | root | U.S. Pat. No. 5,401,836 |
| SbPRP1 | roots | Suzuki et al., Plant Mol. Biol, 21: 109-119, 1993. |
| AtPRP1; AtPRP3 | roots; root hairs | http://salus.medium.edu/mmg/tierney/html |
| RD2 gene | root cortex | http://www2.cnsu.edu/ncsu/research |
| TobRB7 gene | root vasculature | http://www2.cnsu.edu/ncsu/research |
| AtPRP4 | leaves; flowers; lateral root | http://salus.medium.edu/mmg/tierney/html |
| | primordla | |
| seed-specific genes | seed | Simon, et al., Plant Mol. Biol. 5: 191, 1985; Scofield, |
| | | et al., J. Biol. Chem. 262: 12202, 1987.; |
| | | Baszczynski, et al., Plant Mol. Biol. 14: 633, 1990. |
| Brazil Nut albumin | seed | Pearson, et al., Plant Mol. Biol. 18: 235-245, 1992. |
| legumin | seed | Ellis, et al., Plant Mol. Biol. 10: 203-214, 1988. |
| glutelin (rice) | seed | Takaiwa, et al., Mol. Gen. Genet. 208: 15-22, 1986; |
| | | Takaiwa, et al., FEBS Letts. 221: 43-47, 1987. |
| zein | seed | Matzke et al Plant Mol Biol, 14(3): 323-32 1990 |
| napA | seed | Stalberg, et al, Planta 199: 515-519, 1996. |
| wheat LMW and HMW glutenin-1 | edosperm | Mol Gen Genet 216: 81-90, 1989; NAR 17: 461-2, |
| | | 1989 |
| wheat SPA | seed | Albanl et al, Plant Cell, 9: 171-184, 1997 |
| wheat α, β, γ-glladins | edosperm | EMBO 3: 1409-15, 1984 |
| barley Itr1 promoter | endosperm | |
| barley B1, C, D, hordeln | endosperm | Theor Appl Gen 98: 1253-62, 1999; Plant J 4: 343-55, |
| | | 1993; Mol Gen Genet 250: 750-60, 1996 |
| barley DOF | endosperm | Mena et al, The Plant Joumal, 116(1): 53-62, 1998 |
| blz2 | endosperm | EP99106056.7 |
| synthetic promoter | endosperm | Vicente-Carbajosa et al., Plant J. 13: 629-640, 1998. |
| rice prolamin NRP33 | endosperm | Wu et al, Plant Cell Physiology 39(8) 885-889, 1998 |
| rice α-globulin Glb-1 | endosperm | Wu et al, Plant Cell Physiology 39(8) 885-889, 1998 |
| rice OSH1 | embryo | Sato et al, Proc. Natl. Acad, Scl. USA, 93: 8117-8122, |
| | | 1996 |
| rice α-globulin REB/OHP-1 | endosperm | Nakase et al. Plant Mol. Biol. 33: 513-522, 1997 |
| rice ADP-glucose PP | endosperm | Trans Res 6: 157-68, 1997 |
| mize ESR gene family | endosperm | Plant J 12: 235-46, 1997 |
| sorgum γ-kaflrin | endosperm | PMB 32: 1029-35, 1996 |
| KNOX | embryo | Postma-Haarsma et al, Plant Mol. Biol. 39: 257-71, |
| | | 1999 |
| rice oleosin | embryo and aleuron | Wu et al, J. Biochem., 123: 386, 1998 |
| sunflower oleosin | seed (embryo and dry seed) | Cummins, et al., Plant Mol. Biol, 19: 873-876, 1992 |
| LEAFY | shoot meristem | Weigel et al., Cell 69: 843-859, 1992. |
| Arabidopsis thallana knat1 | shoot meristem | Accession number AJ131822 |
| Malus domestica kn1 | shoot meristem | Accession number Z71981 |
| CLAVATA1 | shoot meristem | Accession number AF049870 |
| stigma-specific genes | stigma | Nasrallah, et al., Proc. Natl. Acad. Sci. USA 85: |
| | | 5551, 1988; Trick, et al., Plant mol. Biol. 15: 203. |
| | | 1990 |
| class I patatin gene | tuber | Liu et al., Plant Mol. Biol. 153: 386-395, 1991. |
| PCNA rice | meristem | Kosugi et al, Nucleic Acids Research 19: 1571-1576, |
| | | 1991; Kosugl S. and Ohashi Y, Plant Cell 9: 1607-1619, 1997 |
| Pea TubA1 tubulin | Dividing cells | Stotz and Long, Plant Mol. Biol. 41, 601-614. 1999 |
| Arabidopsis cdc2a | cycling cells | Chung and Parish, FEBS Lett, 3; 362(2): 215-9, 1995 |
| Arabidopsis Rop1A | Anthers; mature pollen + pollen | Li et al. 1998 Plant Physiol 118, 407-417. |
| | tubes | |
| Arabidopsis AtDMC1 | Melosis-associated | Kllmyuk and Jones 1997 Plant J. 11, 1-14. |
| Pea PS-IAA4/5 and PS-IAA6 | Auxin-Inducible | Wong et al. 1996 Plant J. 9, 587-599. |
| Pea famesyltransferase | Meristematic tissues; phloem near | Zhou et al. 1997 Plant J. 12, 921-930 |
| | growing tissues; light- and sugar- | |
| | repressed | |
| Tobacco (N. sylvestris) cyclin | Dividing cells/meristematic tissue | Trehin et al. 1997 Plant Mol. Biol. 35, 667-672. |
| B1;1 | | |
| Catharanthus roseus | Dividing cells/meristematic tissue | Ito et al. 1997 Plant J. 11, 983-992 |
| Mitotic cyclins CYS (A-type) and | | |
| CYM (B-type) | | |
| Arabidopsis cyc1 At (=cyc B1; 1) | Dividing cells/meristematic tissue | Shaul et al. 1996 Proc. Natl. Acad. Sci. U.S.A 93, |
| and cyc3aAt(A-type) | | 4868-4872. |
| Arabidopsis tef1 promoter box | Dividing cells/meristematic tissue | Reged et al. 1995 Mol. Gen. Genet. 248, 703-711. |
| Catharanthus roseus cyc07 | Dividing cells/meristematic tissue | Ito et al. 1994 Plant Mol. Biol. 24, 863-878. |

II: EXEMPLARY CONSTITUTIVE PROMOTERS

| | EXPRESSION | |
|---|---|---|
| GENE SOURCE | PATTERN | REFERENCE |
| Actin | constitutive | McElroy et al, Plant Cell, 2: 163-171, 1990 |
| CAMV 35S | constitutive | Odell et al, Nature, 313: 810-812, 1985 |
| CaMV 19S | constitutive | Nilsson et al., Physoiol. Plant. 100: 456-462, 1997 |
| GOS2 | constitutive | de Pater et al, Plant J. 2: 837-844, 1992 |
| ubiquitin | constitutive | Christensen et al, Plant Mol. Biol. 18: 675-689, 1992 |
| rice cyclophilin | constitutive | Buchholz et al, Plant Mol Biol. 25: 837-843, 1994 |
| maize histone H3 | constitutive | Lepetit et al, Mol. Gen. Genet. 231: 276-285, 1992 |
| alfalfa histone H3 | constitutive | Wu et al., Nucleic Acids Res. 17: 3057-3063, 1989; |
| | | Wu et al., Plant Mol. Biol. 11: 641-649, 1988 |
| actin 2 | constitutive | An et al, Plant J. 10(1); 107-121, 1996 |

III: EXEMPLARY STRESS-INDUCIBLE PROMOTERS

| NAME | STRESS | REFERENCE |
|---|---|---|
| P5CS (delta(1)-pyrroline-5- | salt, water | Zhang et al. Plant Science. 129: 81-89, 1997 |
| carboxylate syntase) | | |
| cor15a | cold | Hajela et al., Plant Physiol. 93: 1246-1252, 1990 |
| cor15b | cold | Wlihelm et al., Plant Mol Biol. 23: 1073-1077, 1993 |
| cor15a (−305 to +78 nt) | cold, drought | Baker et al., Plant Mol Biol. 24: 701-713, 1994 |
| rd29 | salt, drought, cold | Kasuga et al., Nature Biotechnology 18: 287-291, 1999 |
| heat shock proteins, Including | heat | Barros et al., Plant Mol Biol 19: 665-75, 1992. Marrs et |
| artificial promoters containing | | al., Dev Genet. 14: 27-41, 1993. Schoffl et al., Mol Gen |
| the heat shock element (HSE) | | Gent, 217: 246-53, 1989. |
| smHSP (small heat shock | heat | Waters et al, J Experimental Botany 47: 325-338, 1996 |
| proteins) | | |
| wcs120 | cold | Ouellet et al., FEBS Lett. 423: 324-328, 1998 |
| cl7 | cold | Kirch et al., Plant Mol Biol 33: 897-909, 1997 |
| Adh | cold, drought, hypoxia | Dolferus et al., Plant Physiol 105: 1075-87, 1994 |
| pwsl18 | water, salt and drought | Joshee et al., Plant Cell Physiol 39: 64-72, 1998 |
| ci21A | cold | Schneider et al., Plant Physiol 113: 335-45, 1997 |
| Trg-31 | drought | Chaudhary et al., Plant Mol Biol 30: 1247-57, 1996 |
| osmotin | osmotic | Raghothama et al., Plant Mol Biol 23: 1117-28, 1993 |
| IapA | wounding, enviromental | WO99/03977 University of California/INRA |

IV: EXEMPLARY PATHOGEN-INDUCIBLE PROMOTERS

| NAME | PATHOGEN | REFERENCE |
|---|---|---|
| RB7 | Root-knot nematodes | US5760386 - North Carolina State University; |
| | (Meloidogyne spp.) | Opperman et al (1994) Science 263: 221-23. |
| PR-1, 2, 3, 4, 5, 8, 11 | fungal, viral, bacterial | Ward et al (1991) Plant Cell 3: 1085-1094; Reiss et al |
| | | 1996; Lebel at al (1998), Plant J, 16(2): 223-33; |
| | | Melchers et al (1994), Plant J, 5(4): 469-80; Lawton et al |
| | | (1992), Plant Mol Biol, 19(5): 735-43. |
| HMG2 | nematodes | WO9503690 - Virginia Tech Intellectual Properties Inc. |
| Abl3 | Cyst nematodes (Heterodera spp.) | Unpublished |
| ARM1 | nematodes | Barthels et al., (1997) The Plant Cell 9, 2119-2134. |
| | | WO 98/31822 - Plant Genetic Systems |
| Att0728 | nematodes | Barthels et al., (1997) The Plant Cell 9, 2119-2134. |
| | | PCT/EP98/07761 |
| Att1712 | nematodes | Barthels et al., (1997) The Plant Cell 9, 2119-2134. |
| | | PCT/EP98/07761 |
| Gst1 | Different types of pathogens | Strittmatter et al (1996) Mol. Plant-Microbe Interact. 9, |
| | | 68-73. |
| LEMMI | nematodes | WO 92/21757 - Plant Genetic Systems |
| CLE | geminivirus | PCT/EP99/03445 - CINESTAV |
| PDF1.2 | Fungal including Altemaria | Manners et al (1998), Plant Mol Biol, 38(6): 1071-80. |
| | brassicicola and Botrytis cinerea | |
| Thi2.1 | Fungal - Fusarium oxysporum f | Vignutelll et al (1998) Plant J; 14(3): 285-95 |
| | sp. matthiolae | |
| DB#226 | nematodes | Bird and Wilson (1994) Mol. Plant-Microbe Interact., 7, |
| | | 419-42 |
| | | WO 95.322888 |
| DB#280 | nematodes | Bird and Wilson (1994) Mol. Plant-Microbe Interact., 7, |
| | | 419-42 |
| | | WO 95.322888 |
| Cat2 | nematodes | Niebel et al (1995) Mol Plant Microbe Interact 1995 |
| | | May-Jun; 8(3): 371-8 |
| □Tub | nematodes | Aristizabal et al (1998), 8^{th} International Congress on |
| | | Plant-Microbe Interaction, Knoxville US B-29 |
| SHSP | nematodes | Fenoll at al (1997) In: Cellular and molecular aspects |
| | | of plant-nematode Interactions. Kluwer Academic, C. Fenoll, |
| | | F. M. W. Grundler and S. A. Ohl (Eds.), |
| Tsw12 | nematodes | Fenoll et al (1997) In: Cellular and molecular aspects |
| | | of plant-nematode Interactions. Kluwer Academic, C. Fenoll, |
| | | F. M. W. Grundler and S. A. Ohl (Eds.) |
| Hs1(pro1) | nematodes | WO 98/122335 - Jung |
| NsLTP | viral, fungal, bacterial | Molina & Garc 1a-Olmedo (1993) FEBS Lett, |
| | | 316(2): 119-22 |
| RIP | viral, fungal | Tumer et al (1997) Proc Natl Acad Sci USA, |
| | | 94(8): 3866-71 |

Those skilled in the art will be aware of additional promoter sequences and terminator sequences which may be suitable for use in performing the invention. Such sequences may readily be used without any undue experimentation.

In the context of the current invention, “ectopic expression” or “ectopic overexpression”of a gene or a protein are conferring to expression patterns and/or expression levels of said gene or protein normally not occurring under natural conditions, more specifically is meant increased expression and/or increased expression levels. Ectopic expression can be achieved in a number of ways including operably linking of a coding sequence encoding said protein to an isolated homologous or heterologous promoter in order to create a chimeric gene and/or operably linking said coding sequence to its own isolated promoter (i.e. the unisolated promoter naturally driving expression of said protein) in order to create a recombinant gene duplication or gene multiplication effect. With “ectopic co-expression” is meant the ectopic expression or ectopic overexpression of two or more genes or proteins. The same or, more preferably, different promoters are used to confer ectopic expression of said genes or proteins.

Preferably, the promoter sequence used in the context of the present invention is operably linked to a coding sequence or open reading frame (ORF) encoding a cyclin D protein or a homologue, derivative and/or an immunologically active fragment thereof as defined supra.

Preferred promoter sequences of the invention include the alfalfa histone histone H3.2 constitutive promoter (Robertson et al., 1996) and the alfalfa Cdc2MsF M-phase-specific promoter as outlined in Examples 7 and 8.

“Downregulation of expressions” as used herein means lowering levels of gene expression and/or levels of active gene product and/or levels of gene product activity. Decreases in expression may be accomplished by e.g. the addition of coding sequences or parts thereof in a sense orientation (if resulting in co-suppression) or in an antisense orientation relative to a promoter sequence and furthermore by e.g. insertion mutagenesis (e.g. T-DNA insertion or transposon insertion) or by gene silencing strategies as described by e.g. Angell and Baulcombe (1998—WO9836083), Lowe et al. (1989—WO9853083), Lederer et al. (1999—WO9915682) or Wang et al. (1999—WO9953050). Genetic constructs aimed at silencing gene expression may have the nucleotide sequence of said gene (or one or more parts thereof) contained therein in a sense and/or antisense orientation relative to the promoter sequence. Another method to downregulate gene expression comprises the use of ribozymes.

Modulating, including lowering, the level of active gene products or of gene product activity can be achieved by administering or exposing cells, tissues, organs or organisms to said gene product, a homologue, derivative and/or immunologically active fragment thereof. Immunomodulation is another example of a technique capable of downregulation levels of active gene product and/or of gene product activity and comprises administration of or exposing to or expressing antibodies to said gene product to or in cells, tissues, organs or organisms wherein levels of said gene product and/or gene product activity are to be modulated. Such antibodies comprise “plantibodies”, single chain antibodies, IgG antibodies and heavy chain camel antibodies as well as fragments thereof.

Modulating, including lowering, the level of active gene products or of gene product activity can futhermore be achieved by administering or exposing cells, tissues, organs or organisms to an agonist of said gene product or the activity thereof. Such agonists include proteins (comprising e.g. kinases and proteinases) and chemical compounds identified according to the current invention as described supra.

In the context of the invention the term “agonist” refers to a substance that can be either a protagonist or an antagonist, i.e. can have either positive or negative effects, can be an enhancer or an inhibitor or a modulator as well.

In the context of the current invention is envisaged the downregulation of the expression of a cyclin D gene as defined higher. Preferably said cyclin D gene is a plant cyclin D gene, more specifically MTCYCDM. The invention further comprises downregulatlon of levels of a cyclin D protein or of a cylin D activity whereby said cyclin D protein has been defined supra. Preferably said cyclin D protein is a plant cyclin D, more specifically MtCycDm.

By “modifying cell fate and/or plant development and/or plant morphology and/or biochemistry and/or physiology” is meant that one or more developmental and/or morphological and/or biochemical and/or physiological characteristics of a plant is altered by the performance of one or more steps pertaining to the invention described herein.

“Cell fate” refers to the cell-type or cellular characteristics of a particular cell that are produced during plant development or a cellular process therefor, in particular during the cell cycle or as a consequence of a cell cycle process.

“Plant development” or the term “plant developmental characteristic” or similar term shall, when used herein, be taken to mean any cellular process of a plant that is involved in determining the developmental fate of a plant cell, in particular the specific tissue or organ type into which a progenitor cell will develop. Cellular processes relevant to plant development will be known to those skilled in the art. Such processes include, for example, morphogenesis, photomorphogenesis, shoot development, root development, vegetative development, reproductive development, stem elongation, flowering, and regulatory mechanisms involved in determining cell fate, in particular a process or regulatory process involving the cell cycle.

“Plant morphology” or the term “plant morphological characteristic” or similar term will, when used herein, be understood by those skilled in the art to refer to the external appearance of a plant, including any one or more structural features or combination of structural features thereof. Such structural features include the shape, size, number, position, colour, texture, arrangement, and patternation of any cell, tissue or organ or groups of cells, tissues or organs of a plant, including the root, stem, leaf, shoot, petiole, trichome, flower, petal, stigma, style, stamen, pollen, ovule, seed, embryo, endosperm, seed coat, aleurone, fibre, fruit, cambium, wood, heartwood, parenchyma, aerenchyma, sieve element, phloem or vascular tissue, amongst others.

“Plant biochemistry” or the term “plant biochemical characteristic” or similar term will, when used herein, be understood by those skilled in the art to refer to the metabolic and catalytic processes of a plant, including primary and secondary metabolism and the products thereof, including any small molecules, macromolecules or chemical compounds, such as but not limited to starches, sugars, proteins, peptides, enzymes, hormones, growth factors, nucleic acid molecules, celluloses, hemicelluloses, calloses, lectins, fibres, pigments such as anthocyanins, vitamins, minerals, micronutrients, or macronutrients, that are produced by plants.

“Plant physiology” or the term “plant physiological characteristic” or similar term will, when used herein, be understood to refer to the functional processes of a plant, including developmental processes such as growth, expansion and differentiation, sexual development, sexual reproduction, seed set, seed development, grain filling, asexual reproduction, cell division, dormancy, germination, light adaptation, photosynthesis, leaf expansion, fibre production, secondary growth or wood production, amongst others; responses of a plant to externally-applied factors such as metals, chemicals, hormones, growth factors, environment and environmental stress factors (eg. anoxia, hypoxia, high temperature, low temperature, dehydration, light, daylength, flooding, salt, heavy metals, amongst others), including adaptive responses of plants to said externally-applied factors.

With “adverse growth conditions” or “stress conditions” is meant, when used herein, all conditions hampering normal plant growth or plant development or decreasing plant growth rate. Said conditions occur when e.g. sufficient amounts of a nutrient are lacking or when environmental stress factors are applied.

Means for introducing recombinant DNA into plant tissue or cells include, but are not limited to, transformation using CaCl₂ and variations thereof, in particular the method described by Hanahan (1983), direct DNA uptake into protoplasts (Krens et al, 1982; Paszkowski et al, 1984), PEG-mediated uptake to protoplasts (Armstrong et al, 1990) microparticle bombardment, electroporation (Fromm et al., 1985), microinjection of DNA (Crossway et al., 1986), microparticle bombardment of tissue explants or cells (Christou et al, 1988; Klein et at, 1992), vacuum-infiltration of tissue with nucleic acid, or in the case of plants, T-DNA-mediated transfer from Agrobacterium to the plant tissue as described essentially by An et al. (1 985), Dodds et al., (1985), Herrera-Estrella et al (1983a, 1983b, 1985). Methods for transformation of monocotyledonous plants are well known in the art and include Agrobacterium-mediated transformation (Cheng et al., 1997—WO9748814; Hansen 1998—WO9854961; Hiei et al., 1994—WO9400977; Hiei et al., 1998—WO9817813; Rikilshi et al., 1999—WO9904618; Saito et al., 1995—WO9506722), microprojectile bombardment (Adams et al., 1999—U.S. Pat. No. 5,969,213; Bowen et al., 1998—U.S. Pat. No. 5,736,369; Chang et al., 1994—WO9413822; Lundquist et al., 1999—U.S. Pat. No. 5,874,265/U.S. Pat. No. 5,990,390; Vasil and Vasil, 1995—U.S. Pat. No. 5,405,765. Walker et al., 1999—U.S. Pat. No. 5,955,362), DNA uptake (Eyal et al., 1993—WO9318168), microinjection of Agrobacterium cells (von Holt, 1994—DE4309203) and sonication (Finer et al., 1997—U.S. Pat. No. 5,693,512).

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed.

Examples of microparticles suitable for use in such systems include 1 to 5 μm gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein, means a process by which shoots and roots are developed sequentially from meristematic centres.

The term “ernbryogenesis”, as used herein, means a process by which shoots and roots develop together in a concerted fashion (not sequentially), whether from somatic cells or gametes.

Preferably, the plant is produced according to the inventive method is transfected or transformed with a genetic sequence, or amenable to the introduction of a protein, by any art-recognized means, such as microprojectile bombardment, microinjection, Agrobacterium-mediated transformation (including in planta transformation), protoplast fusion, or electroporation, amongst others. Most preferably said plant is produced by Agrobacterium-mediated transformation.

Agrobactetium-mediated transformation or agrolistic transformation of plants, yeast, moulds or filamentous fungi is based on the transfer of part of the transformation vector sequences, called the T-DNA, to the nucleus and on integration of said T-DNA in the genome of said eukaryote.

With “Agrobacterium” is meant a member of the Agrobacteriaceae, more preferably Agrobactedium or Rhizobacterium and most preferably Agrobacterium tumefaciens. With “T-DNA”, or transferred DNA, is meant that part of the transformation vector flanked by T-DNA borders which is, after activation of the Agrobacterium vir genes, nicked at the T-DNA borders and is transferred as a single stranded DNA to the nucleus of an eukaryotic cell.

When used herein, with “T-DNA borders”, “T-DNA border region”, or “border region”are meant either right T-DNA border (RB) or left T-DNA border (LB). Such a border comprises a core sequence flanked by a border inner region as part of the T-DNA flanking the border and/or a border outer region as part of the vector backbone flanking the border. The core sequences comprise 22 bp in case of octopine-type vectors and 25 bp in case of nopaline-type vectors.

With “T-DNA transformation vector” or “T-DNA vector” is meant any vector encompassing a T-DNA sequence flanked by a right and left T-DNA border consisting of at least the right and left border core sequences, respectively, and used for transformation of any eukaryotic cell.

With “T-DNA vector backbone sequence” or “T-DNA vector backbone sequences” is meant all DNA of a T-DNA containing vector that lies outside of the T-DNA borders and, more specifically, outside the nicking sites of the border core imperfect repeats. The current invention includes optimized T-DNA vectors such that vector backbone integration in the genome of a eukaryotic cell is minimized or absent. With “optimized T-DNA vector” is meant a T-DNA vector designed either to decrease or abolish transfer of vector backbone sequences to the genome of a eukaryotic cell. Such T-DNA vectors are known to the one familiar with the art and include those described by Hanson et al. (1999) and by Stuiver et al. (1999—WO9901563).

The current invention clearly considers the inclusion of a DNA sequence encoding a cyclin D, homologue, derivative or immunologically active fragment thereof as defined supra, in any T-DNA vector comprising binary transformation vectors, super-binary transformation vectors, co-integrate transformation vectors, Ri-derived transformation vectors as well as in T-DNA carrying vectors used in agrolistic transformation. Preferably, said cyclin D is a plant cyclin D, more specifically the alfalfa MtCycDm. With “binary transformation vector” is meant a T-DNA transformation vector comprising:
(a) a T-DNA region comprising at least one gene of interest and/or at least one selectable marker active in the eukaryotic cell to be transformed; and
(b) a vector backbone region comprising at least origins of replication active in E. coli and Agrobacterium and markers for selection in E. coli and Agrobacterium.

The T-DNA borders of a binary transformation vector can be derived from octopine-type or nopaline-type Ti plasmids or from both. The T-DNA of a binary vector is only transferred to a eukaryotic cell in conjunction with a helper plasmid.

With “helper plasmid” is meant a plasmid that is stably maintained in Agrobacterium and is at least carrying the set of vir genes necessary for enabling transfer of the T-DNA. Said set of vir genes can be derived from either octopine-type or nopaline-type Ti plasmids or from both.

With “super-binary transformation vector” is meant a binary transformation vector additionally carrying in the vector backbone region a vir region of the Ti plasmid pTiBo542 of the super-virulent A. tumefaciens strain A281 (EP0604662, EP0687730). Super-binary transformation vectors are used in conjunction with a helper plasmid. With “co-integrate transformation vector” is meant a T-DNA vector at least comprising:
(a) a T-DNA region comprising at least one gene of interest and/or at least one selectable marker active in plants; and
(b) a vector backbone region comprising at least origins of replication active in Escherichia coli and Agrobactetium, and markers for selection in E. coli and Agrobacterium, and a set of vir genes necessary for enabling transfer of the T-DNA.

The T-DNA borders and said set of vir genes of a said T-DNA vector can be derived from either octopine-type or nopaline-type Ti plasmids or from both.

With “Ri-derived plant transformation vector” is meant a binary transformation vector in which the T-DNA borders are derived from a Ti plasmid and said binary transformation vector being used in conjunction with a ‘helper’ Ri-plasmid carrying the necessary set of vir genes.

As used herein, the term “selectable marker gene” or “selectable marker” or “marker for selection” includes any gene which confers a phenotype on a cell in which it is expressed to facilitate the identification and/or selection of cells which are transfected or transformed with a gene construct of the invention or a derivative thereof. Suitable selectable marker genes contemplated herein include the ampicillin resistance (Amp^{r}) tetracycline resistance gene (Tc^{r}), bacterial kanamycin resistance gene (Kan^{r}), phosphinothricin resistance gene, neomycin phosphotransferase gene (npflI), hygromycin resistance gene, β-glucuronidase (GUS) gene, chloramphenicol acetyitransferase (CAT) gene, green fluorescent protein (gfp) gene (Haseloff et al, 1997), and luciferase gene, amongst others.

With “agrolistics”, “agrolistic transformation” or “agrolistic transfer” is meant here a transformation method combining features of Agrobacterium-mediated transformation and of biolistic DNA delivery. As such, a T-DNA containing target plasmid is co-delivered with DNA/RNA enabling in planta production of VirD1 and VirD2 with or without VirE2 (Hansen and Chilton 1996; Hansen et al. 1997; Hansen and Chilton 1997—WO9712046).

The present invention further describes an approach to remove from transformed cells a stably integrated foreign DNA sequence by recombination involving a recombinase and recombination sites.

With “foreign DNA” is meant any DNA sequence that is introduced in the host's genome by recombinant techniques. Said foreign DNA includes e.g. a T-DNA sequence or a part thereof such as the T-DNA sequence comprising the selectable marker in an expressible format. Foreign DNA furthermore include intervening DNA sequences as defined supra.

With “recombination event” is meant either a site-specific recombination event or a recombination event effected by transposon ‘jumping’.

With “recombinase” is meant either a site-specific recombinase or a transposase. With “recombination site” is meant either site-specific recombination sites or transposon border sequences.

With “site specific recombination event” is meant an event catalyzed by a system generally consisting of three elements: a pair of DNA sequences (the site-specific recombination sequences or sites) and a specific enzyme (the site-specific recombinase). The site-specific recombinase catalyzes a recombination reaction only between two site-specific recombination sequences depending on the orientation of the site-specific recombination sequences. Sequences intervening between two site-specific recombination sites will be inverted in the presence of the site-specific recombinase when the site-specific recombination sequences are oriented in opposite directions relative to one another (i.e. inverted repeats). If the site-specific recombination sequences are oriented in the same direction relative to one another (i.e. direct repeats), then any intervening sequences will be deleted upon interaction with the site-specific recombinase. Thus, if the site-specific recombination sequences are present as direct repeats at both ends of a foreign DNA sequence integrated into a eukaryotic genome, such integration of said sequences can subsequently be reversed by interaction of the site-specific recombination sequences with the corresponding site specific recombinase.

A number of different site specific recombinase systems can be used including but not limited to the Cre/lox system of bacteriophage P1, the FLP/FRT system of yeast, the Gin recombinase of phage Mu, the Pin recombinase of E. coli, the PinB, PinD and PinF from Shigella, and the R/RS system of the pSR1 plasmid. Recombinases generally are integrases, resolvases or flippases. Also dual-specific recombinases can be used in conjunction with direct or indirect repeats of two different site-specific recombination sites corresponding to the dual-specific recombinase (WO99/25840). The two preferred site-specific recombinase systems are the bacteriophage P1 Cre/lox and the yeast insertion, gene stacking, etc. (WO99/25821) and for resolution of complex T-DNA integration patterns or for excision of a selectable marker (WO99/23202).

Although the site-specific recombination sequences must be linked to the ends of the DNA to be excised or to be inverted, the gene encoding the site specific recombinase may be located elsewhere. For example, the recombinase gene could already be present in the eukaryote's DNA or could be supplied by a later introduced DNA fragment either introduced directly into cells, through crossing or through cross-pollination. Alternatively, a substantially purified recombinase protein could be introduced directly into the eukaryotic cell, e.g. by micro-injection or particle bombardment. Typically, the site-specific recombinase coding region will be operably linked to regulatory sequences enabling expression of the site-specific recombinase in the eukaryotic cell.

With “recombination event effected by transposon jumping” or “transposase-mediated recombination” is meant a recombination event catalyzed by a system consisting of three elements: a pair of DNA sequences (the transposon border sequences) and a specific enzyme (the transposase). The transposase catalyzes a recombination reaction only between two transposon border sequences which are arranged as inverted repeats.

A number of different transposon/transposase systems can be used including but not limited to the Ds/Ac system, the Spm system and the Mu system. These systems originate from corn but it has been shown that at least the Ds/Ac and the Spm system also function in other plants (Fedoroff et al. 1993, Schlappi et al. 1993, Van Sluys et al. 1987). Preferred are the Ds- and the Spm-type transposons which are delineated by 11 bp- and 13 bp- border sequences, respectively.

Although the transposon border sequences must be linked to the ends of the DNA to be excised, the gene encoding the transposase may be located elsewhere. For example, the recombinase gene could already be present in the eukaryote's DNA or could be supplied by a later introduced DNA fragment either introduced directly into cells, through crossing or through cross-pollination. Alternatively, a substantially purified transposase protein could be introduced directly into cells, e.g. by microinjection or by particle bombardment.

As part of the current invention, transposon border sequences are included in a foreign DNA sequence such that they lie outside said DNA sequence and transform said DNA into a transposon-like entity that can move by the action of a transposase.

As transposons often reintegrate at another locus of the host's genome, segregation of the progeny of the hosts in which the transposase was allowed to act might be necessary to separate transformed hosts containing e.g. only the transposon footprint and transformed hosts still containing the foreign DNA.

In performing the present invention, the genetic element is preferably induced to mobilize, such as, for example, by the expression of a recombinase protein in the cell which contacts the integration site of the genetic element and facilitates a recombination event therein, excising the genetic element completely, or alternatively, leaving a “footprint”, generally of about 20 nucleotides in length or greater, at the original integration site. Those hosts and host parts that have been produced according to the inventive method can be identified by standard nucleic acid hybridization and/or amplification techniques to detect the presence of the mobilizable genetic element or a gene construct comprising the same. Alternatively, in the case of transformed host cells, tissues, and hosts wherein the mobilizable genetic element has bene excised, it is possible to detect a footprint in the genome of the host which has been left following the excision event, using such techniques. As used herein, the term “footprint” shall be taken to refer to any derivative of a mobilizable genetic element or gene construct comprising the same as described herein which is produced by excision, deletion or other removal of the mobilizable genetic element from the genome of a cell transformed previously with said gene construct. A footprint generally comprises at least a single copy of the recombination loci or transposon used to promote excision. However, a footprint may comprise additional sequences derived from the gene construct, for example nucleotide sequences derived from the left border sequence, right border sequence, origin of replication, recombinase-encoding or transposase-encoding sequence if used, or other vector-derived nucleotide sequences. Accordingly, a footprint is identifiable according to the nucleotide sequence of the recombination locus or transposon of the gene construct used, such as, for example, a sequence of nucleotides corresponding or complementary to a lox site or fit site.

The term “cell cycle” means the cyclic biochemical and structural events associated with growth and with division of cells, and in particular with the regulation of the replication of DNA and mitosis. Cell cycle includes phases called: G0, Gap1 (G1), DNA synthesis (S), Gap2 (G2), and mitosis (M). Normally these four phases occur sequentially, however, the cell cycle also includes modified cycles wherein one or more phases are absent resulting in modified cell cycle such as endomitosis, acytokinesis, polyploidy, polyteny, and endoreduplication.

The term “cell cycle interacting protein”, “cell cycle protein” or “cell cycle control protein”as denoted herein means a protein which exerts control on or regulates or is required for the cell cycle or part thereof of a cell, tissue, organ or whole organism and/or DNA replication. It may also be capable of binding to, regulating or being regulated by cyclin dependent kinases or their subunits. The term also includes peptides, polypeptides, fragments, variant, homologs, alleles or precursors (eg preproproteins or preproteins) thereof.

Cell cycle control proteins and their role in regulating the cell cycle of eukaryotic organisms are reviewed in detail by John (1981) and the contributing papers therein (Norbury and Nurse 1992; Nurse 1990; Ormrod and Francis 1993) and the contributing papers therein (Doemer et al. 1996; Elledge 1996; Francis and Halford 1995; Francis et al. 1998; Hirt et al. 1991; Mironov et al. 1999) which are incorporated by reference.

The term “cell cycle control genes” refers to any gene or mutant thereof which exerts control on or are required for: chromosomal DNA synthesis and for mitosis (preprophase band, nuclear envelope, spindle formation, chromosome condensation, chromosome segregation, formation of new nuclei, formation of phragmoplast, duplication of microtubule-organizing center, etc) melosis, cytokinesis, cell growth, endoreduplication, cell cycle control genes are also all genes exerting control on the above: homologues of CDKs, cyclins, E2Fs, Rb, CKI, Cks, and also any genes which interfere with the above, cyclin D, cdc25, Wee1, Nim1, MAP kinases, etc.

More specifically, cell cycle control genes are all genes involved in the control of entry and progression through S phase. They include; not exclusively, genes expressing “cell cycle control proteins” such as cyclin dependent kinases (CDK), cyclin dependent kinase inhibitors (CKI), D, E and A cyclins, E2F and DP transcription factors, pocket proteins, CDC7/DBF4 kinase, CDC6, MCM2-7, Orc proteins, cdc45, components of SCF ubiquitin ligase, PCNA, DNA-polymerase.

The term “cell cycle control protein” include cyclins A, B, C, D and E including CYCA1;1, CYCA2;1, CYCA3;1, CYCB1;1, CYCB1;2, CYC B2;2, CYCD1;1, CYCD2;1, CYCD3;1, and CYCD4;1 (Evans et al. 1983; Francis et al. 1998; Labbe et al. 1989; Murray and Kirschner 1989; Renaudin et al. 1996; Soni et al. 1995; Sorrell et al. 1999; Swenson et al. 1986) cyclin dependent kinase inhibitor (CKI) proteins such as ICK1 (Wang et al. 1997), FL39, FL66, FL67 (PCT/EP98/05895), Sic1, Far1, Rum1, p21, p27, p57, p16, p15, p18, p19 (Elledge 1996; Pines 1995), p14 and p14ARF; p13suc1 or CKS1At (De Veylder et al. 1997; Hayles and Nurse 1986) and nim-1 (Russell and Nurse 1987a; Russell and Nurse 1987b; Fantes 1989; Russell and Nurse 1986; Russell and Nurse 1987a; Russell and Nurse 1987b) homologues of Cdc2 such as Cdc2MsB (Hirt et al. 1993) CdcMs kinase (Bogre et al. 1997) cdc2 T14Y15 phosphatases such as Cdc25 protein phosphatase or p80cdc25 (Bell et al. 1993; Elledge 1996; Kumagal and Dunphy 1991; Russell and Nurse 1986) and Pyp3 (Elledge 1996) cdc2 protein kinase or p34cdc2 (Colasanti et al. 1991; Feiler and Jacobs 1990; Hirt et al. 1991; John et al. 1989; Lee and Nurse 1987; Nurse and Bissett 1981; Ormrod and Francis 1993) cdc2a protein kinase (Hemerly et al. 1993) cdc2 T14Y15 kinases such as wee1 or p107wee1 (Elledge 1996; Russell and Nurse 1986; Russell and Nurse 1987a; Russell and Nurse 1987b; Sun et al. 1999) mik1 (Lundgren et al. 1991) and myt1 (Elledge 1996); cdc2 T161 kinases such as Cak and Civ (Elledge 1996); cdc2 T161 phosphatases such as Kap1 (Elledge 1996); cdc28 protein kinase or p34cdc28 (Nasmyth 1993; Reed et al. 1985) p40MO15 (Fesquet et al. 1993; Poon et al. 1993) chk1 kinase (Zeng et al. 1998) cds1 kinase (Zeng et al. 1998) growth-associated H1 kinase (Labbe et al. 1989; Lake and Salzman 1972; Langan 1978; Zeng et al. 1998) MAP kinases described by (Binarova et al. 1998; Bögre et al. 1999; Calderini et al. 1998; Wilson et al. 1999).

Other cell cycle control proteins that are involved in cyclin D-mediated entry of cells into G1 from G0 include pRb (Xie et al. 1996; Huntley et al. 1998) E2F, RIP, MCM7 and potentially the pRb-like proteins p107 and p130.

Other cell cycle control proteins that are involved in the formation of a pre-replicative complex at one or more origins of replication, such as, but not limited to, ORC; CDC6, CDC14, RPA and MCM proteins or in the regulation of formation of this pre-replicative complex, such as, but not limited to, the CDC7, DBF4 and MBF proteins.

For the present purpose, the term “cell cycle control protein” shall further be taken to include any one or more of those proteins that are involved in the turnover of any other cell cycle control protein, or in regulating the half-life of said other cell cycle control protein. The term “protein turnover” is to include all biochemical modifications of a protein leading to the physical or functional removal of said protein. Although not limited to these, examples of such modifications are phosphorylation, ubiquitination and proteolysis. Particularly preferred proteins which are involved in the proteolysis of one or more of any other of the above-mentioned cell cycle control proteins include the yeast-derived and animal-derived proteins, Skp1, Skp2, Rub1, Cdc20, cullins, CDC23, CDC27, CDC16, and plant-derived homologues thereof (Cohen-Fix and Koshland 1997; Hochstrasser 1998; Krek 1998; Lisztwan et al. 1998) and Plesse et al in (Francis et al. 1998)).

For the present purpose, the term “cell cycle control genes” shall further be taken to include any one or more of those gene that are involved in the transcriptional regulation of cell cycle control gene expression such as transcription factors and upstream signal proteins. Additional cell cycle control genes are not excluded.

For the present purpose, the term “cell cycle control genes” shall further be taken to include any cell cycle control gene or mutant thereof, which is affected by environmental signals such as for instance stress, nutrients, pathogens, or by intrinsic signals such as the animal mitogens or the plant hormones (auxins, cytokinins, ethylene, gibberellic acid, abscisic acid and brassinosteroids).

The term “cell cycle progression” refers to the process of passing through the different cell cycle phases. The term “cell cycle progression rate” accordingly refers to the speed at which said cell cycle phases are run through or the time spans required to complete said cell cycle phases.

With “two-hybrid assay” is meant an assay that is based on the observation that many eukaryotic transcription factors comprise two domains, a DNA-binding domain (DB) and an activation domain (AD) which, when physically separated (i.e. disruption of the covalent linkage) do not effectuate target gene expression. Two proteins able to interact physically with one of said proteins fused to DB and the other of said proteins fused to AD will re-unite the DB and AD domains of the transcription factor resulting in target gene expression. The target gene in the yeast two-hybrid assay is usually a reporter gene such as the β-galactosidase gene. Interaction between protein partners in the yeast two-hybrid assay can thus be quantified by measuring the activity of the reporter gene product (Bartel and Fields 1997). Alternitavely, a mammalian two-hybrid system can be used which includes e.g. a chimeric green fluorescent protein encoding reporter gene (Shioda et al., 2000).

The term “fragment of a sequence” or “part of a sequence” means a truncated sequence of the original sequence referred to. The truncated sequence (nucleic acid or protein sequence) can vary widely in length; the minimum size being a sequence of sufficient size to provide a sequence with at least a comparable function and/or activity or the original sequence referred to, while the maximum size is not critical. In some applications, the maximum size usually is not substantially greater than that required to provide the desired activity and/or function(s) of the original sequence. Typically, the truncated amino acid or nucleotide sequence will range from about 5 to about 60 amino acids in length. More typically, however, the sequence will be a maximum of about 50 amino acids in lenght, preferably a maximum of about 60 amino acids. It is usually desirable to select sequences of at least about 10, 12 or 15 amino acids or nucleotides, up to a maximum of about 20 or 25 amino acids or nucleotides.

Furthermore, folding simulations and computer redesign of structural motifs of the protein of the invention can be performed using appropriate computer programs (Olszewski et al. 1996, Hoffman et al. 1995). Computer modeling of protein folding can be used for the conformational and energetic analysis of detailed peptide and protein models (Monge et al. 1995, Renouf and Hounsell, 1995). In particular, the appropriate programs can be used for the identification of interactive sites of the ICK and cyclin-dependent kinases, its ligand or other interacting proteins by computer assisted searches for complementary peptide sequences (Fassina and Melli 1994). Further appropriate computer systems for the design of protein and peptides are described in the prior art, for example in Berry and Brenner (1994), Wodak (1987), Pabo and Suchanek (1986). The results obtained form the above-described computer analysis can be used for, e.g. the preparation of peptidomimetics of the protein of the invention or fragments thereof. Such pseudopeptide analogues of the natural amino acid sequence of the protein may very efficiently mimic the parent protein (Benkirane et al. 1996). For example, incorporation of easily available achiral e-amino acid residues into a protein of the invention or a fragment thereof results in the substitution of amino bonds by polymethylene units of an aliphatic chain, thereby providing a convenient strategy for constructing a peptidomimetic (Baneriee et al. 1996). Superactive peptidomimetic analogues of small peptide hormones in other systems are described in the prior art (Zhang et al. 1996). Appropriate peptidomimetics of the protein of the present invention can also be identified by the synthesis of peptidomimetic combinatorial libraries through successive amine alkylation and testing the resulting compounds, e.g., for their binding, kinase inhibitory and/or immunlogical properties. Methods for the generation and use of peptidomimetic combinatioral libraries are described in the prior art, for example in Ostresh et al. (1996) and Domer et al. (1996). Furthermore, a three-dimensional and/or crystallographic structure of the protein of the invention can be used for the design of peptidomimetic inhibitors of the biological activity of the protein of the invention (Rose et al. 1996, Rutenber et al. 1996).

The compounds to be obtained or identified in the methods of the invention can be compounds that are able to bind to any of the nucleic acids, peptides or proteins of the invention. Other interesting compounds to be identified are compounds that modulate the expression of the genes or the proteins of the invention in such a way that either the expression of said gene or protein is enhanced or decreased by the action of said compound. Alternatively the compound can exert his action by directly or indirectly enhancing or decreasing the activity of any of the proteins of the invention.

Said compound or plurality of compounds may be comprised in, for example, samples, e.g., cell extracts from, e.g., plants, animals or microorganisms. Furthermore, said compound(s) may be known in the art but hitherto not known to be capable of suppressing or activating cell cycle interacting proteins. The reaction mixture may be a cell free extract of may comprise a cell or tissue culture. Suitable set ups for the method of the invention are known to the person skilled in the art and are, for example, generally described in Alberts et al., Molecular Biology of the Cell, third edition (1994), in particular Chapter 17. The plurality of compounds may be, e.g., added to the reaction mixture, culture medium or injected into the cell.

If a sample containing a compound or a plurality of compounds is identified in the method of the invention, then it is either possible to isolate the compound from the original sample identified as containing the compound capable of acting as an agonist, or one can further subdivide the original sample, for example, if it consists of a plurality of different compounds, so as to reduce the number of different substances per sample and repeat the method with the subdivisions of the original sample. Depending on the complexity of the samples, the steps described above can be performed several times, preferably until the sample identified according to the method of the invention only comprises a limited number of or only one substance(s). Preferably said sample comprises substances or similar chemical and/or physical properties, and most preferably said substances are identical. Preferably, the compound identified according to the above described method or its derivative is further formulated in a form suitable for the application in plant breeding or plant cell and tissue culture.

Methods for extraction and/or production of pure silica or SiO₂ from rice seed peels or husks are known in the art (e.g. Gorthy and Pudukottah 1999) and units for production of SiO₂ from rice seed peels are being set up (visit e.g. http://bisnis.doc.gov/bisnis/leads/990604sp.htm). SiO₂ has many applications including electronics, perfume industry and pharmacology.

The present invention is further described by reference to the following non-limiting figures and examples.

### FIGURE LEGENDS

FIG. 1. Amino acid sequence alignment of different plant cyclins D including the plant cyclin of the invention, MtCycDm. Identical amino acids are boxed. Gaps are introduced to allow maximum sequence alignment. The cyclin box region is indicated with a box at the level of the amino acid numbering ruler. Am: Anthirhinum majus; At: Arabidopsis thaliana; Cr: Chenopodium rubrum; Ht: Helianthus tuberosus; Mt: Medicago sativa; Nt: Nicotlana tabacum; Zm: Zea mays. The amino acid sequence corresponding to the Medicago sativa cyclin box is represented in SEQ ID NO 28.

FIG. 2. Phylogenetic tree of plant D-type cyclins including the plant cyclin of the invention, MtCycDm. The tree was drawn on the basis of the complete amino acid sequences of the indicated cyclins and using the PROTDIST software (Felsenstein 1993; (http://evolution.genetics.washinqton.edu/phylip. Html)). Am: Anthirrhinum malus; At: Arabidopsis thaliana; Cr: Chenopodium rubrum; Ht: Helianthus tuberosus; Le: Lycopersicon esculentum; Ms: Medicago sativa; Mt: Medicago truncatula; Nt: Nicotiana tabacum; Zm: Zea mays.

FIG. 3. Phylogenetic tree of plant D-type cyclins including the plant cyclin of the invention, MtCycDm. The tree was drawn on the basis of the cyclin box region of the indicated cyclins and using the PROTDIST software (Felsenstein, 1993 (http://evolution.genetics.washington.edu/phylip.html)). Am: Anthirrhinum majus, At: Arabidopsis thaliana; Cr: Chenopodium rubrum; Ht: Helianthus tuberosus; Le: Lycopersicon esculentum; Ms: Medicago sativa; Mt: Medicago truncatula; Nt: Nicotiana tabacum; Zm: Zea mays.

FIG. 4. Amino acid sequence alignment of different plant D-type cyclins as in FIG. 1. PEST-regions are, however, boxed instead of identical amino acids. The amino acid sequence corresponding to the Medicago saliva PEST-region is represented in SEQ ID NO 30.

FIG. 5. Interaction in a yeast two-hybrid assay of alfalfa Cdc2-type or Cdc2-related kinases and different D-type cyclins and myosin. The different Cdc2Ms kinase cDNAs are cloned in the pGBT9 bait vector and the cDNAs of the interacting partners in the pGAD424 activation domain vector. (A) Growth of PJ69-4a yeast cells transformed with expression vectors comprising the indicated protein coding sequences on the agar surface of a selective medium lacking tryptophan, leucine, adenine and histidine. The relative growth values were also determined in liquid cultures and are numerically indicated. (B) Quantification of β-galactosidase enzyme acitivity of yeast strains described in (A).

FIG. 6. Expression of MtCycDm and Cdc2MsF genes during the cell cycle in hydroxyurea-synchronized alfalfa A2 cell suspension. RNA gel blot analysis was performed with gene-specific probes hybridized to total RNA isolated from cell samples collected at different stages of the progressing cell cycle. The relative density of the hybridization signals were plotted against said cell cycle stages.

FIG. 7. Expression of MtCycDm and Cdc2MsF during the cell cycle in hydroxyurea-synchronized alfalfa A2 cell suspensions as determined by RT-PCR on the same RNA samples as described in FIG. 6.

FIG. 8. Distribution of the activities of the different indicated Cdc2-type or Cdc2-related alfalfa kinases during the 8, G2 and M cell cycle phases of synchronized alfalfa cell cultures.

FIG. 9. Immunolocalization of the alfalfa mitosis-specific Cdc2MsF. DAPI staining of nuclear material is represented as insets in each immunolabelling image. The white colour represents maximum signal intensity. The bar located in metaphase inset represents 25 μM. Cdc2MsF is located on the preprophase band (A), prophase spindle (B), metaphase spindle (C) and phragmoplast (D).

FIG. 10. Plasmid map of pCAMBIA-type binary plant transformation vector pC3300MtcycDm containing the MtCycDm open reading frame operably linked to the constitutive alfalfa histone H3.2 gene promoter.

FIG. 11. Plasmid map of pCAMBIA-type binary plant transformation vector pC3300promF-MtcycDm containing the MtCycDm open reading operably linked to the M-phase specific alfalfa Cdc2MsF gene promoter.

FIG. 12. Interactions between Medicago different Cdc2s and Medicago cyclin-dependent kinase inhibitor (CKI Mt). Protein-protein interactions are tested in yeast strains containing the CKI Mt open reading frame fused to the GAL4 DNA binding domain and either one of the indicated Medicago Cdc2 open reading frames fused to the GAL4 activation domain (all furtheron in this caption referred to as Medicago fusion protein). “Self-activation” is a control wherein the GAL4 activation domain is not fused to a Medicago Cdc2 open reading frame. Panel A shows the growth of said yeast strains on selective medium lacking Trp, Leu, His and Ade. Only when two Medicago fusion proteins interact will a yeast strain grow. Panel B is a graphical representation of β-galactosidase activity as a result of LacZ gene expression activated by interaction of two Medicago fusion proteins. Medsa: Medicago sativa; Medtr: Medicago truncatula.

FIG. 13. Interactions between Medicago different cyclins and Medicago cyclin-dependent kinase inhibitor (CKI Mt). Protein-protein interactions are tested in yeast strains containing the CKI Mt open reading frame fused to the GAL4 DNA binding domain and either one of the indicated Medicago cyclin open reading frames fused to the GAL4 activation domain (all furtheron in this caption referred to as Medicago fusion protein). “Self-activation” is a control wherein the GAL4 activation domain is not fused to a Medicago cyclin open reading frame. Panel A shows the growth of said yeast strains on selective medium lacking Trp, Leu, His and Ade. Only when two Medicago fusion proteins interact will a yeast strain grow. Panel B is a graphical representation of β-galactosidase activity as a result of LacZ gene expression activated by interaction of two Medicago fuslon proteins.

FIG. 14. Effect of recombinant (His)₆-CKI Mt on histone H1 kinase activity of different Medicago protein complexes. Protein complexes containing the indicated Medicago proteins were obtained by immunoprecipitation. Histone H1 kinase activity of the isolated complexes was determined in the absence or presence of different amounts of recombinant (His)₆-CKI Mt. Relative histone H1 kinase activities are depicted whereby a 100% activity is the histone H1 kinase activity of each separate isolated protein complex in the absence of recombinant (His)₆-CKI Mt.

FIG. 15. Effect of recombinant (His)₆-CKI Mt on histone H1 and Medicago retinoblastoma protein (MsRb) kinase activity of isolated Medicago Cdc2MsA/B protein complexes. The protein complex containing the Medicago Cdc2MsA/B kinases was obtained by immunoprecipitation from cells in either S-phase or G2/M phase. Protein kinase activity of the isolated complexes was determined using histone H1 and MsRb as substrates both in the absence or presence of recombinant (His)₆-CKI Mt. Protein kinase activity is given in counts per minute (CPM).

FIG. 16. Alignment of MtCycDm cDNA sequence, with nucleotide sequences of Medicago truncatula ESTs with GenBank accession numbers AW981189 and AW689816. Further schematically indicated are the positions of SEQ ID NOs 16 to 21, all relative to the MtCycDm cDNA sequence.

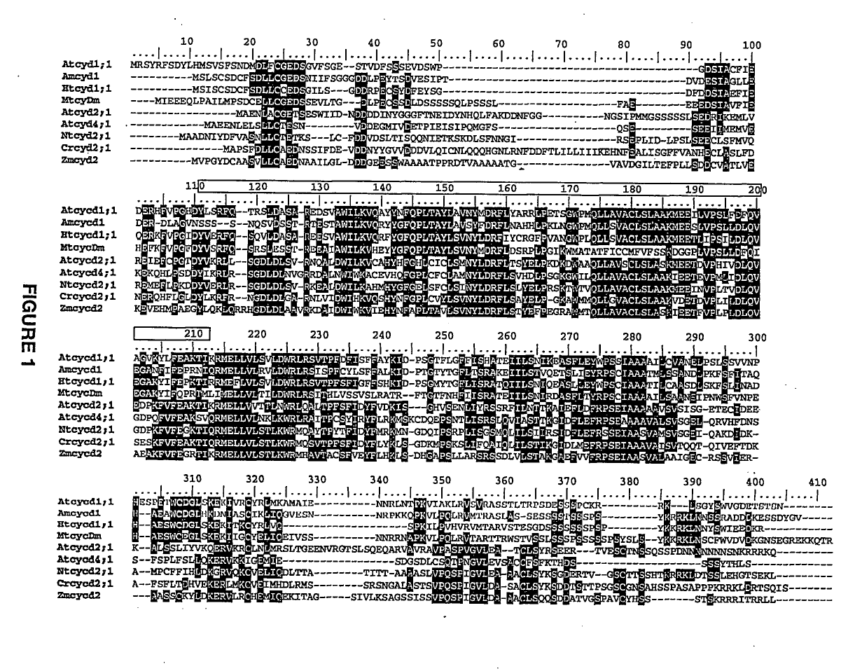

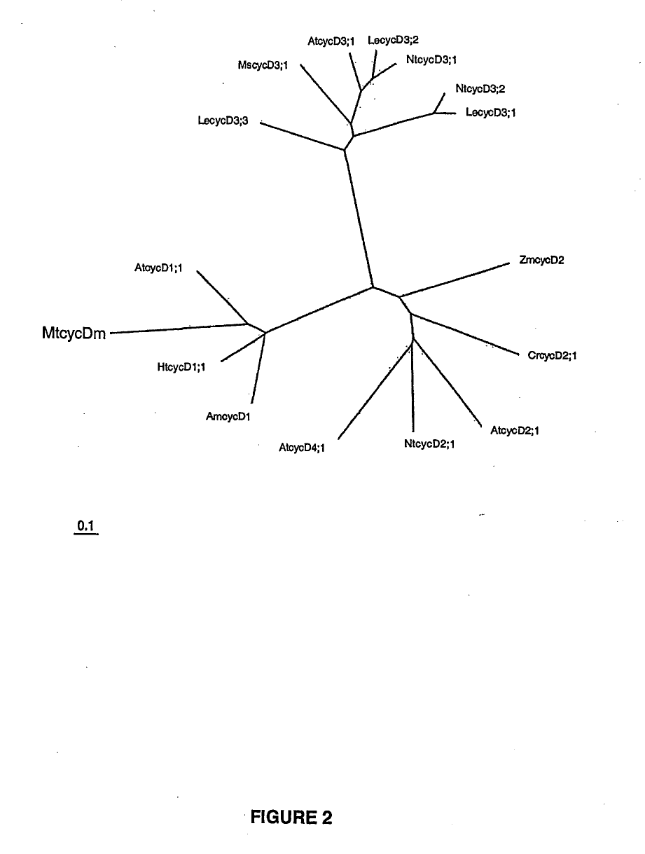

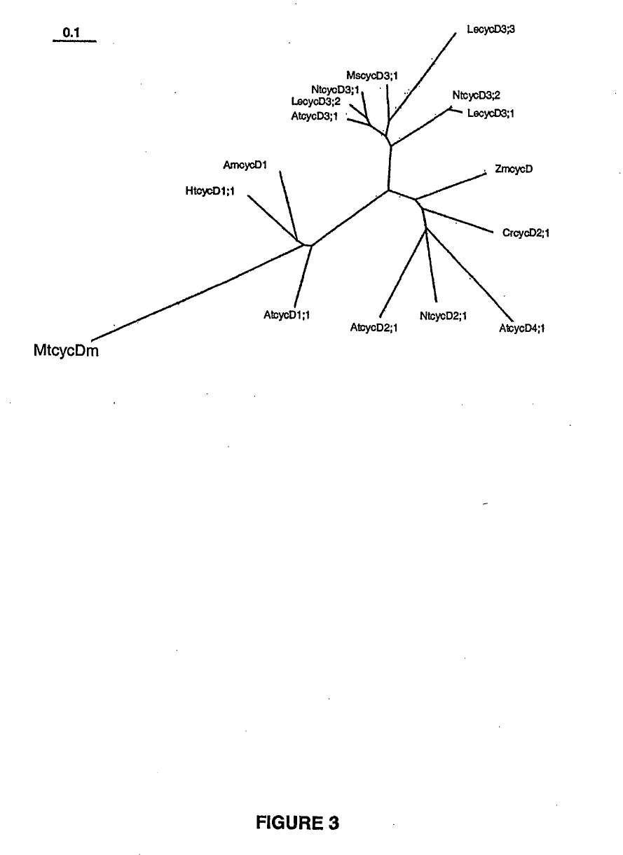

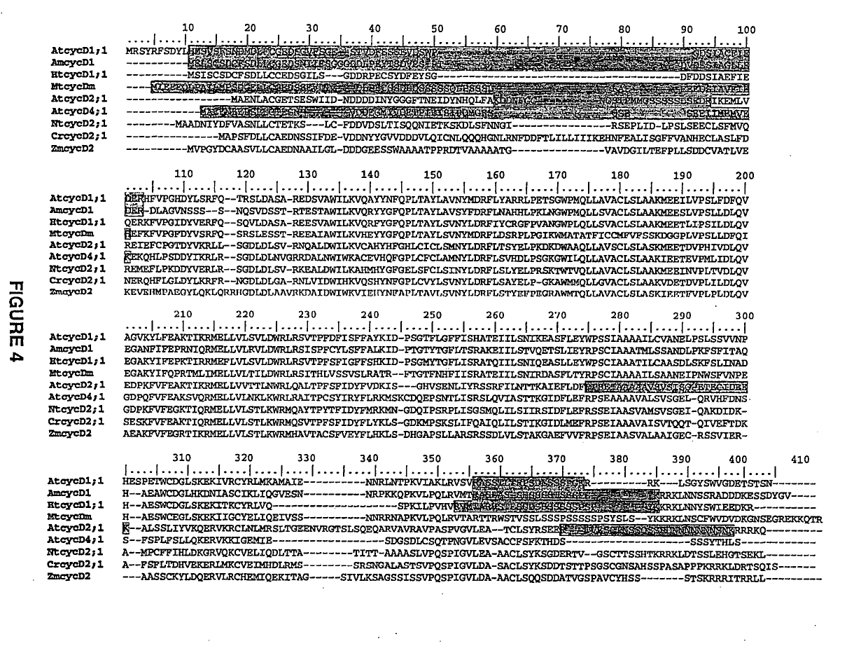

### EXAMPLES

### Example 1

### Identification of a Novel D-Type Cyclin cDNA from M. truncatula

The cDNA library was constructed in the phage λHybriZAP with the synthesis and cloning kit of Stratagene (La Jolla, Calif.). Seedlings of M. truncatula line R108 were grown in aeroponic tanks and inoculated with the wild-type Sinorhizobium meliloti strain Rm41 as described by Hoffmann et al. (1997). Poly-A⁺ mRNA was purified from young root nodules 4 to 8 days after Sinorhizobium infection. The cDNA synthesis of 1.6 μg of poly-A⁺ mRNA was primed by oligo-(dT)-Xhol adapter primer with MMLV-reverse transcriptase while the second strand was synthesized via polymerase I-ribonuclease H coincubation. EcoRI adapter was added to the blunted, double-stranded cDNA, followed by Xhol digestion. CDNAs longer than 400 bp were directionally cloned in EcoRI-Xhol digested λHybriZAP phage vector. The library was excised in vivo according to the manufacturer's instructions and pADGal4-2.1 phagemids carrying individual bacterial clones were obtained.

The M. truncatula cDNA library obtained as described supra was used in a yeast two-hybrid screening for proteins interacting with M. sativa Cdc2MsA, its cDNA cloned between the EcoRI and SalI restriction sites of pBD-Gal4 (Stratagene, La Jolla, Calif.). The yeast two-hybrid screening was performed according to the manufacturer's instructions and yielded 16 positive clones of which three of them were identified as the cyclin D of the invention termed MtCycDm. The transformation of yeast cells was carried out by the PEG/Li acetate method as described by Gietz and Schlesti (1995). The transformation efficiency was calculated to be 8.7×10⁵. The cDNA inserts of positive clones were sequenced by the dideoxy chain termination method (Sanger et al. 1977) using an automated DNA sequencer (Applied Biosystems 373, Applied Blosystems, Foster City, Calif.).

### Example 2

### Specificity of the Interaction of Medicago D-Type Cyclins and Cdc2-Type or Cdc2-Related Kinases.

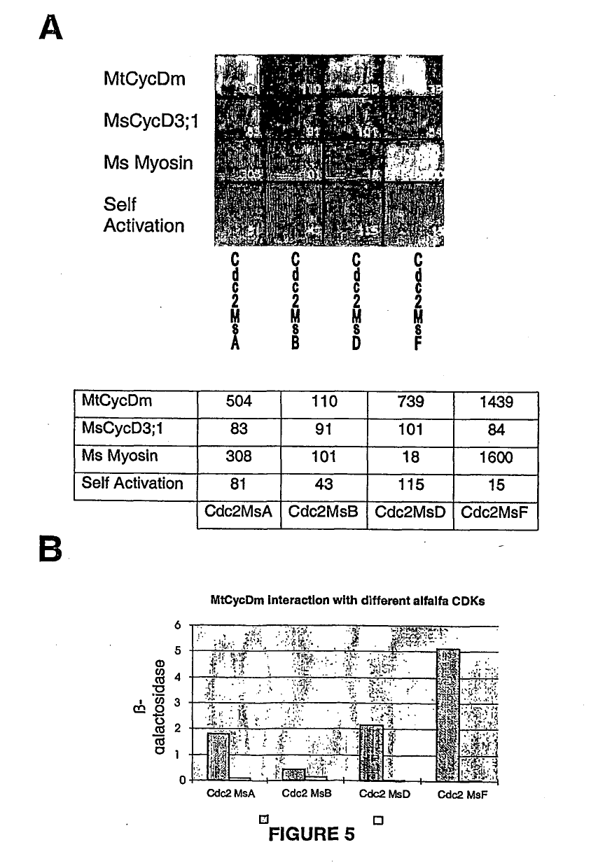

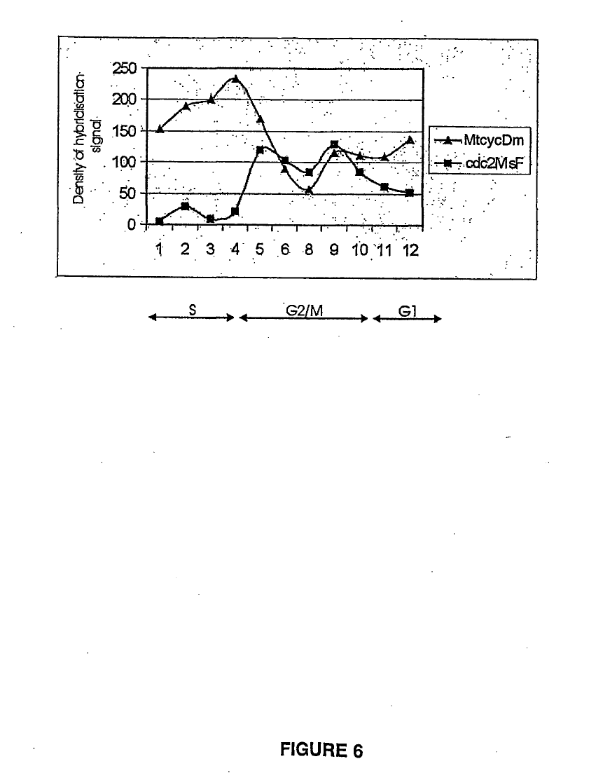

To determine interaction specificity of protein partners potentially interacting with different alfalfa Cdc2-type or Cdc2-related kinases, cDNAs of alfalfa kinases Cdc2MsA, Cdc2MsB, Cdc2MsD and Cdc2MsF were cloned as baits into the pGBT9 vector (Clontech, Palo Alto, Calif.). The cDNAs of the interacting partners MsCycD3;1, MtCycDm and MsMyosin were cloned as preys into the pGAD424 vector (Clontech, Palo Alto, Calif.). The relative growth of PJ69-4A yeast cells on the surface of selective agar medium lacking tryptophan, leucine, adenine and hisitidine and transformed with the different bait-prey combinations is depicted in FIG. 5A. In the same figure the relative growth values as determined in liquid cultures are also numerically indicated. The interaction between MtCycDm and said different alfalfa Cdc2-type or Cdc2-related kinases was furthermore quantified by means of the relative β-galactosidase enzyme activities as shown in FIG. 5B. The data obtained from these experiments indicate a preferential interaction between MtCycDm and Cdc2MsF followed by a less pronounced association of MtCycDm with Cdc2MsD and Cdc2MsA. The binding of MtCycDm with Cdc2MsB is neglible. No significant interaction between MsCycD3;1 and any of the alfalfa Cdc2-type or Cdc2-related kinases was observed. A strong interactions was apparent between MsMyosin and Cdc2MsF, but not with any of the other Cdc2Ms kinases.

### Example 3

### Tissue Culture Systems, Cell Synchronization and Flow Cytometric Analysis

A fast growin cell suspension culture was established from primary callus tissues from in vitro grown plants of alfalfa, M. sativa var. varia cv. Rambler (A2). Cultures containing single cells and small multicellular colonies were maintained in MS medium supplemented with 1 mg/L 2,4-dichlorophenoxyacetic acid (2,4-D) and 0.2 mg/L kinetin (KIN). The cell suspensions were subcultured twice a week.

Cells from regularly subcultured M. varia A2 suspension cultures were treated with 5 mM hydroxyurea (HU) or with 20 μg/mL aphidicolin for 36 h after the last subculture. To release them from the HU or aphidicolin block, cells were pelleted, washed three times, subsequently resuspended in a medium containing 25% (w/w) conditioned MS medium harvested from alfalfa A2 cell suspensions at logarithmic growth phase and further cultured for synchronous growth.

The isolation of nuclei and flow cytometric analysis were performed according to Savouré et al. (1995) using a FACSₛₜₐᵣ^{plus} instrument equipped with a 5W argon ion laser adjusted to an emission at 488 nm. The EtBr fluorescence emission was collected in a second photomultiplier at wavelengths above 580 nm.

### Example 4

### Analysis of MtCycDm and Cdc2MsF Expression by RNA Gel Blot Analysis and by RT-PCR.

Synchronized alfalfa A2 cell suspension cultures were obtained as described in Example 3. The total RNA was extracted according to the freezing phenol method (Maes and Messens 1992) with a slight modification to scale the method to the volume of Eppendorf tubes. Frozen alfalfa suspension cells were homogenized under liquid nitrogen in a small mortar with 0.5 mL of phenol (equilibrated to pH 4.9 with 3M K-acetate). After diluting with 0.5 mL of 1% SDS, the samples were incubated for 15 min at 65° C. and then centrifuged for 10 min in an Eppendorf centrifuge at 4° C. The supernatant was extracted with phenol-chloroform twice and precipitated with one-quarter volume of 8M LiCl for overnight incubation at 4° C. The precipitates were dissolved in 100 μL of diethyl pyrocarbonate-treated water, and a second LiCl precipitation was applied to remove residual DNA contamination. Total RNA was quantified by optical density measurement at 260 nm (Sambrook et al. 1989). Twenty micrograms of total RNA was loaded on 1% formaldehyde gel containing 0.01% ethidium bromide. Transferring RNAs to Hybond D filters (Amersham) was performed with the capillary action technique (Sambrook et al. 1989) and the filters were examined under UV light to verify the efficiency of transfer and to test the quality and quantity of loaded RNA samples. Hybridization of the filters was performed in Rapid-hyb buffer (Amersham) at 65° C. Radiolabeled probes were generated by PCR. The following synthetic oligonucleotides were used for amplifying the nucleotides 206-1005 of the Cdc2MsF cDNA and the nucleotides 198-630 of the MiCycDm cDNA:
Cdc2MsF: Fpri2 seq, 5′-ACCTGCAGCTAAAGATGGGTCTTGTC-3′ (SEQ ID NO 8) and Fpri3 seq, 5′-ATAGAATTCACTTCATGAAGATGATGAA-3′ (SEQ ID NO 9); and
MtCycDm: primer A, 5′-TTGCTGTTTTCATCGAGCAC-3′ (SEQ ID NO 10) and primer B, 5′-GTTGCACGCAAAGAAACTGA-3′ (SEQ ID NO 11).

The amounts of loaded RNA were standardized by hybridization with a probe derived from alfalfa actin cDNA. Hybridization signals were first observed on Kodak X-ray films and then quantified on a STORM phosphoimager (Molecular Dynamics, Sunnyvale, Calif.).

Expression of the MtCycDm and Cdc2MsF genes was also visualized by RT-PCR analysis using the same total RNA samples and synthetic oligonucleotides as described supra. The following conditions were applied for RT-PCR amplification of:
Cdc2MsF:
   an initial denaturation step of 5 min at 95° C.; and
   five initial amplification rounds: 20 sec at 94° C. (denaturation), 30 sec at 50° C. (primer annealing) and 80 sec at 72° C. (elongation); and
   twenty-five additional amplification rounds: 20 sec at 94° C. (denaturation), 30 sec at 56° C. (primer annealing) and 80 sec at 72° C. (elongation).
MtCycDm:
   an initial denaturation step of 4 min at 95° C.; and
   five initial amplification rounds: 20 sec at 94° C. (denaturation), 40 sec at 50° C. (primer annealing) and 50 sec at 72° C. (elongation); and
   twenty-five additional amplification rounds: 20 sec at 94° C. (denaturation), 40 sec at 56° C. (primer annealing) and 50 sec at 72° C. (elongation).

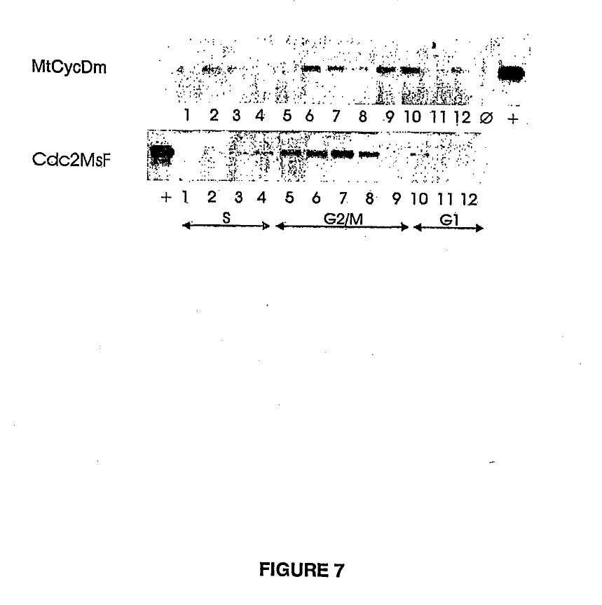

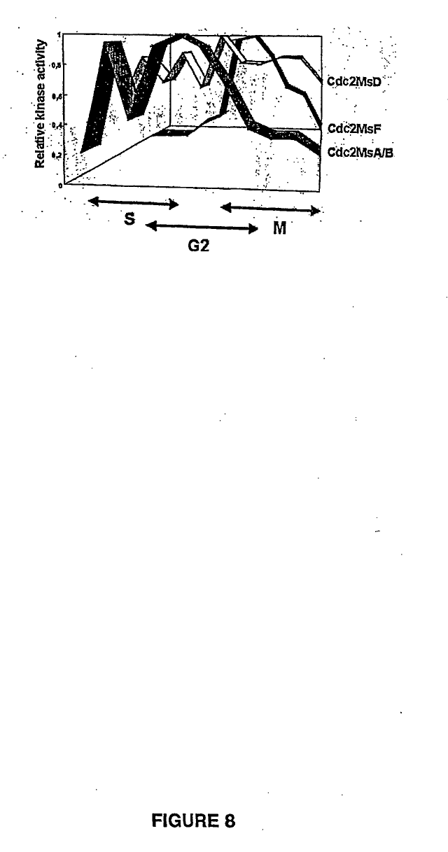

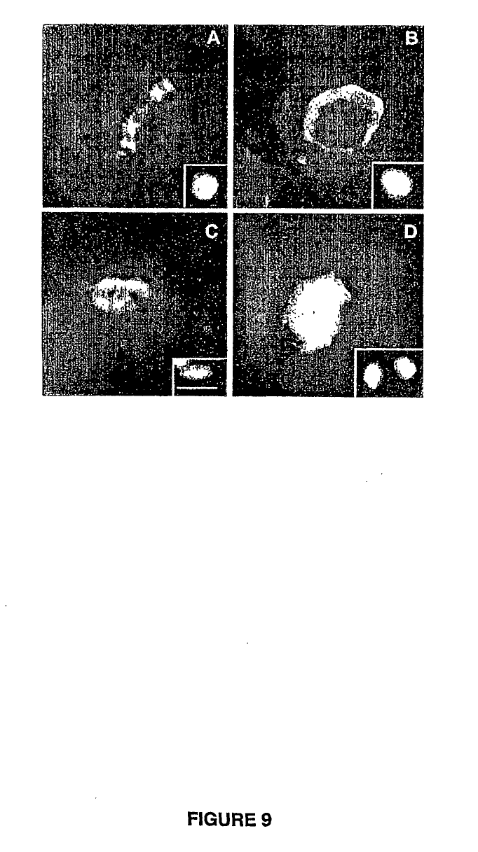

The experimental results (see FIG. 7 and FIG. 8) reveal that both MtCycDm and Cdc2MsF genes are co-expressed during the G2-M phases of the cell cycle.

### Example 5

### Activities of Cdc2-Type or Cdc2-Related Alfalfa Kinases in Synchronized Cell Cultures

Polyclonal antibodies raised against the carboxy-terminal EYFKDIKFVP peptide of alfalfa Cdc2MsA/B (common sequence motif, Hirt et al. 1991, 1993), the carboxy-terminal PYFDSLDKSQY peptide of alfalfa Cdc2MsD or the carboxy-terminal DDLDKTHL peptide of alfalfa Cdc2MsF were raised in rabbits by using synthetic peptides coupled to Keyhole limpet hemocyanin through an additional cysteine residue at their amino-termini.

Crude IgG fractions were separated from whole sera by ammonium sulphate fractionation. The IgG fractions were further purified on peptide-coupled affinity columns as described by Harlow and Lane (1988).

Synchronized alfalfa cells (see Example 3) were homogenized with quartz sand in extraction buffer (25 mM Tris-HCl, 15 mM EGTA, 15 mM p-nitrophenylphosphate, 1 mM dithiothreitol, 60 mM β-glycerophosphate, 0.1% Nonidet P-40, 0.1 mM Na-vanadate, 0.5 mM NaF, 10 μg/mL aprotinin, 10 μg/mL antipain, 10 μg/mL leupeptin, 5 μg/mL pepstatin, 5 μg/mL chymostatin, 1 mM phenylmethylsulfonlylfluoride, pH 7.5) and centrifuged at 25000 g. The protein content was determined by using the Bio-Rad Protein Assay kit (Bradford et al. 1976).

For immunoprecipitation, equal amounts of protein samples in extraction buffer were precleared with 60 μL of 25% (v/v) proteinA-Sepharose beads (Pharmacia) for 1 hr at 4° C. on a rotary shaker. After a short centrifugation, the supernatants were transferred to new Eppendorf tubes containing 2 to 4 μg of anti-carboxy-terminal alfalfa antibodies (anti-Cdc2MsA/B, anti-Cdc2MsF or anti-Cdc2MsD) and incubated at 4° C. for 1 hr. In the following step, 50 μL of 50% (v/v) suspension of proteinA-Sepharose was added, and the tubes were gently shaken for 1 hr at 4° C. Thereafter, the beads were washed three times with RIPA buffer containing 20 mM Tris-HCl, 5 mM EDTA, 2 mM EGTA, 100 mM NaCl, 2 mM NaF, 0.2% Nonidet P-40, 300 μM phenylmethylsulfonyifluoride, 10 μg/mL aprotinin, 10 μg/mL leupeptin, pH 7.4.

Histone H1 kinase reaction mixtures consisted of 50 mM Tris-HCl, 15 mM MgCl2, 5 mM EGTA, 1 mM DTT, 1 mg/mL histone H1 (IIIS, Sigma, St Louis, Mo.) and 10 μCu of [γ-³²P]ATP in 30 μL of total volume. Kinase reactions were started by the addition of 30 μL reaction mixture to the washed proteinA-Sepharose beads (complexed with Cdc2MsA/B or Cdc2MsF) and stopped after 15 min at 28° C. by the addition of 7.5 μL 5× SDS sample buffer. The samples were analyzed by 10% SDS-PAGE (Laemmli 1970) and radioactivity was quantified using a STORM phospoimager (Molecular Dynamics, Sunnyvale, Calif.).

The CTD domain of Arabidopsis thaliana topolsomerase II served as substrate for the Cdc2MsD kinase.

The results of the described experiments are compiled in FIG. 8 clarifying the consecutive action of Cdc2MsA/B, Cdc2MsD and Cdc2MsF during the G2-M phases.

### Example 6

### Immunolocalization of Cdc2MsF

Dense drops of cells were fixed for 1 h at 23° C. in MTSB (5OmM PIPES pH 6.9, 5 mM MgSO₄, 5 mM EGTA), washed with the same buffer and digested for 20 min with 2ml of enzyme mixture (1% w/v cellulase Onozuka R-10, 1% v/v pectinase) in MTSB. Following washing with MTSB, cells were attached to coated slides and were extracted for 20 min with 0.5% Triton-X-100. Rabbit anti-cdc2MsF antibody (see Example 5) was applied (1/200 dilution) for 15 hrs at 4° C. Anti-rabbit FITC conjugated secondary antibody (Sigma, Mo., USA) was used for 1 h at 23° C. with 1/200 dilution after washing the primary antibody. Before the last wash of the secondary antibody, nuclei were stained with 100 ng μl⁻¹ DAPI (4′,6-diamidino-2phenylindole HCl) and cells were mounted with Citifluor (Ted Pella Inc. CA, USA).

### Example 7

### Vector Construction for Overexpression of MtcycDm cDNA Under the Control of a Constitutive Alfalfa Histone H3 Promoter

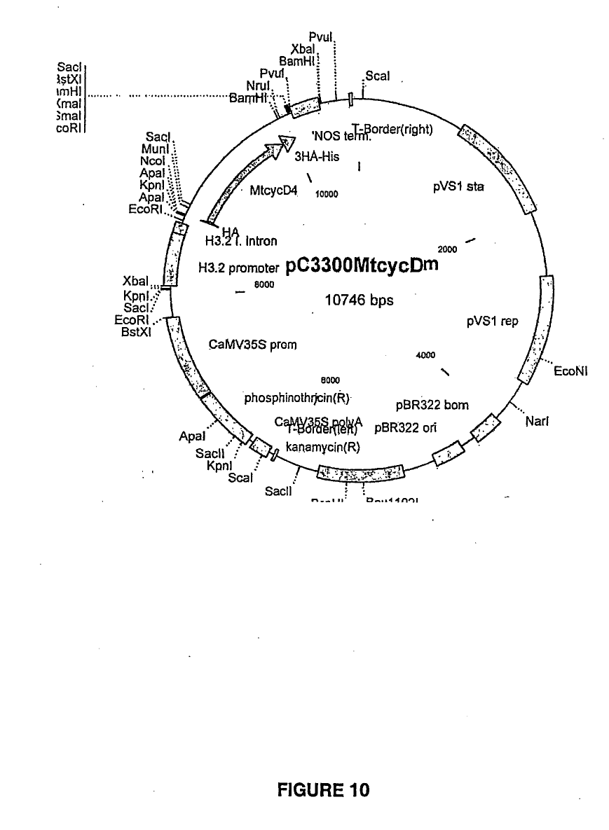

The coding region of MtcycDm was created by polymerase chain reaction using the pAD Gal4.1 MtcycDm as template. Primers were designed to incorporate additional enzyme restriction sites. The upper primer was 5′-GCGCCCATATGAGATCTGCCATGATTGAAGAAGAGC-3′ (SEQ ID NO 12) with Ndel (single underlined) and Bg/II (double underlined) restriction sites, the lower primer 5′-CCGAAGCTTCATATGCCGCGTTTGTTTCTTTTCTCTTC CCTC-3′ (SEQ ID NO 13) with HindIII (single underlined) and Ndel (double underlined) restriction sites. The PCR product was digested with Ndel and ligated into in-frame a pBluescript Sk construct that contain four HA and a polyHis epitope peptides. This cassette was cloned into the Kpnl and SacII restriction sites in the MCS of pH3, with the 5′ terminus of the MtcycDm sequence at the Kpnl site. The resulting plasmid was cut with Spel and SacII restriction enzymes fill-in and ligated to eliminate the unwanted Notl site. This construct was digested with Notl, blunted and ligated into the Smal-digested pCAMBIA3300 vector containing the alfalfa histone H3.2 promoter (Robertson et al. 1996). The resuting vector is called pC3300cycDm (FIG. 10) and is used for plant transformation. Plant species that are transformed are tobacco (N. tabacum; according to Fisher and Guiltinan 1995), alfalfa (M. sativa; according to Trinh et al. 1998) and rice (O. sativa; according to Toki 1997).

### Example 8

### Vector Construction for Overexpression of MtcycDm cDNA Under the Control of a G2/M-Specific Alfalfa cdc2MsF Promoter

The promoter of the cdc2MsF kinase was cloned by PCR from an adaptor-ligated pool of Medicago sativa genomic DNA using adaptor specific (5′-TAT GGA ATT CGC GGC CGC G-3′; SEQ ID NO 14) and cdkF specific (5′-GCG ATT GTT TCA CCA GGT TTC TCC-3′; SEQ ID NO 15) primers. PCR product was end-polished for blunt subcloning. The construct pC3300MtcycDm (see Example 7and FIG. 10) was partially digested with Xbal restriction enzyme, cohesive ends of the linearized plasmid were blunted by Klenow polyrnerase end-filling reaction and self-ligated. Following transformation into E. coli, pC3300MtcycDmX was selected which has a unique Xbal site at position 8077 but lacks the not desired Xbal site at position 10389 of the parental construct.

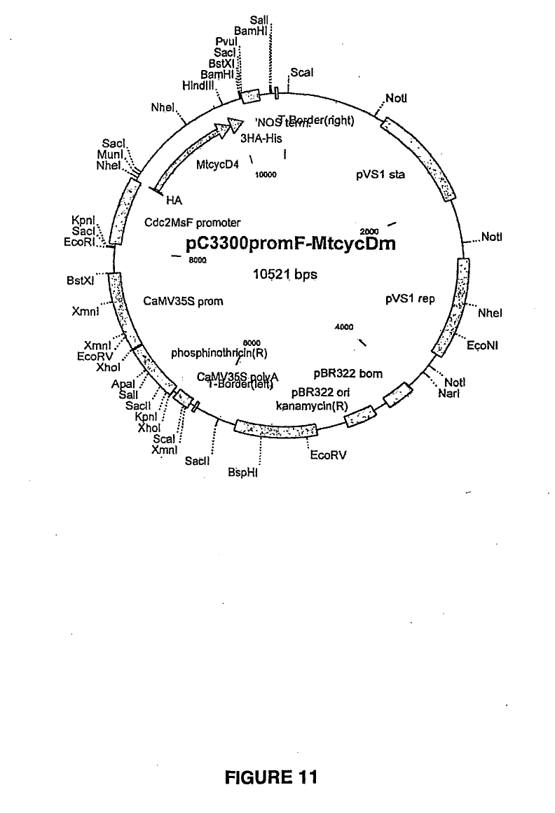

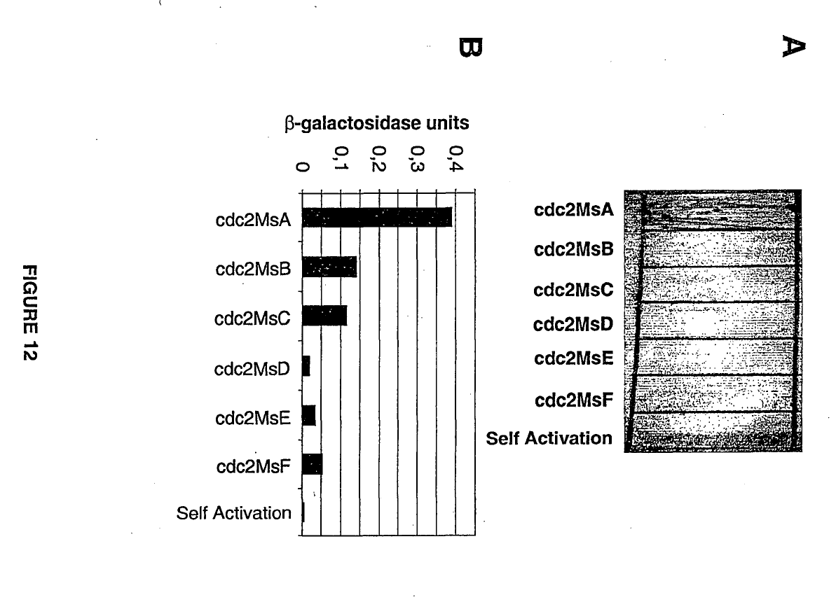

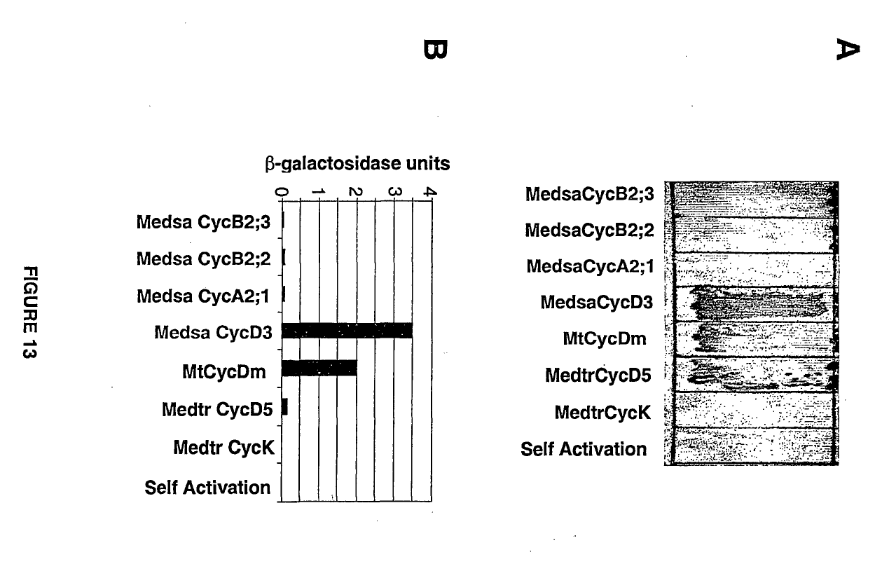

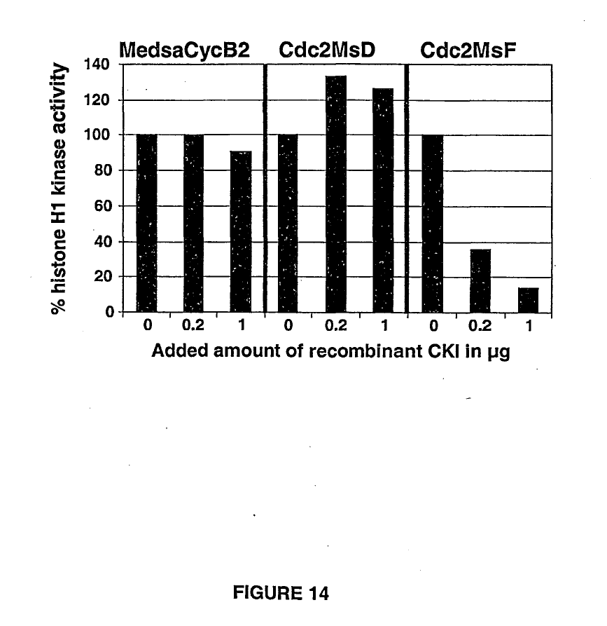

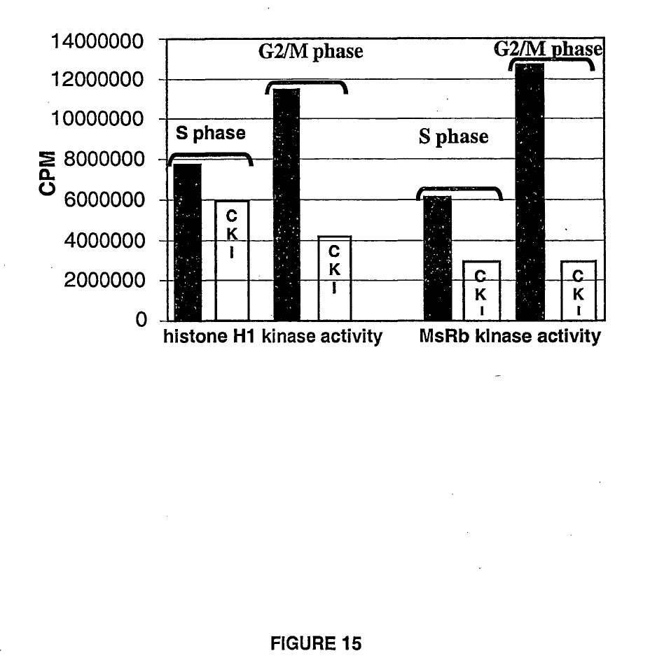

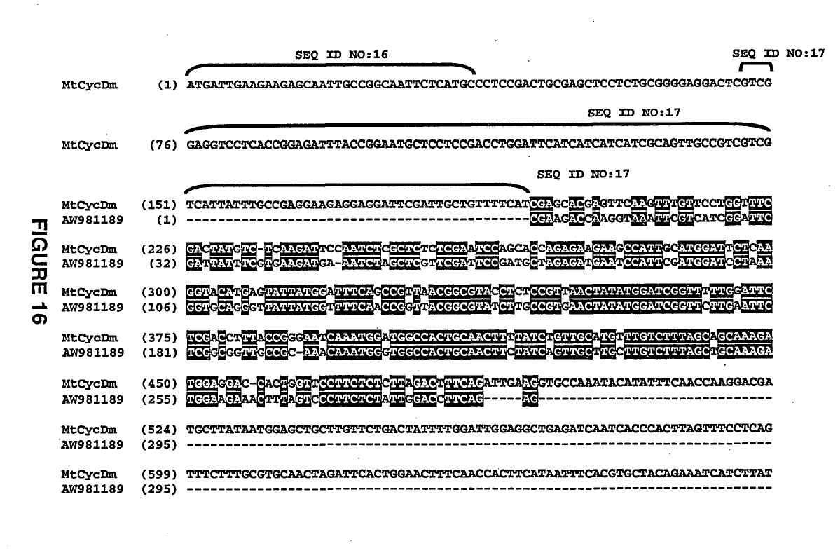

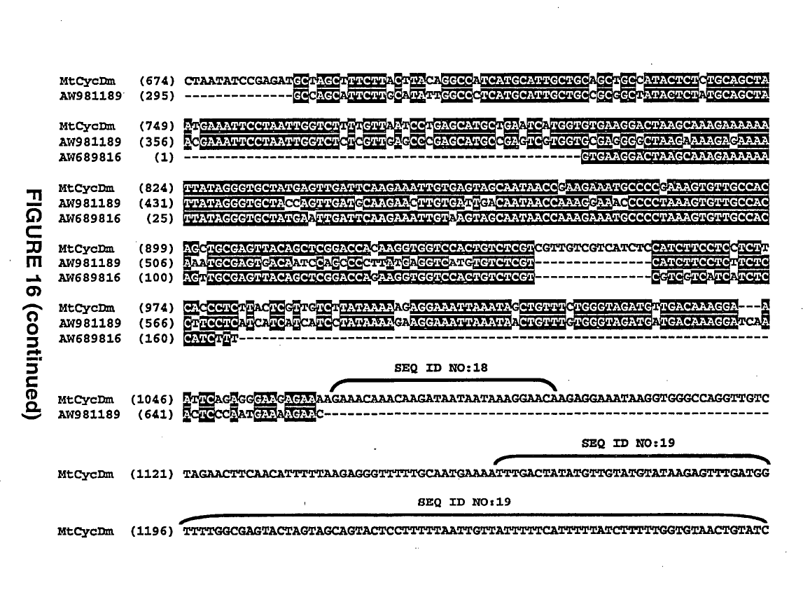

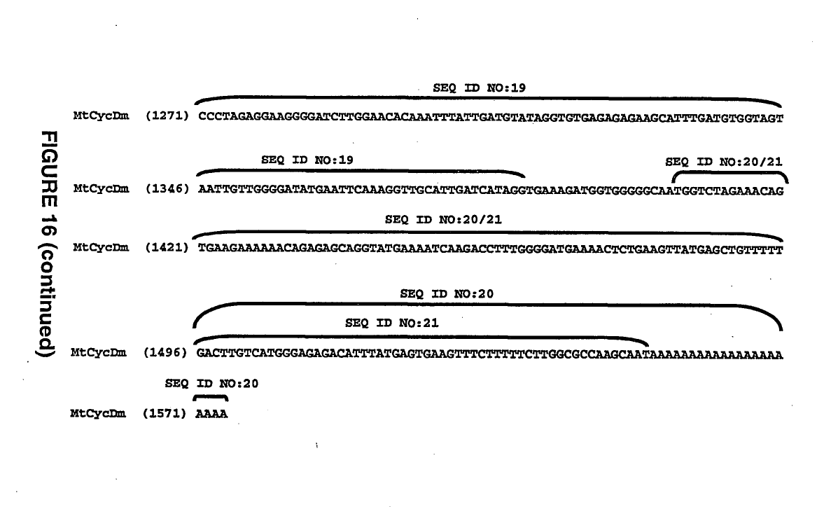

pC3300MtcycDmX was digested with Ncol and Xbal, blunted by end-filling and the vector fragment was purified to eliminate the histone H3 promoter. The cdc2MsF promoter fragment was blunt ligated into this vector and transformed into E.coli. The recombinant product carrying the cdc2MsF promoter in the proper orientation was selected and designated pC3300promF-MtcycDm (FIG. 11). The pC3300promF-MtcycDm vector is used for plant transformation. Plant species that are transformed are tobacco (N. tabacum; according to Fisher and Guiltinan 1995), alfalfa (M. sativa; according to Trinh et al. 1998) and rice (O. sativa; according to Toki 1997).

### Example 9

### Interaction Between Medicago cdc2's, Medicago Cyclins and Medicago Cyclin-Dependent Kinase Inhibitor (CKI) and Kinase Activities of Different Medicago cdc2-Containing Complexes

DNA Manipulations

Restriction endonucleases and DNA modifying enzymes were purchased from Stratagene, Promega and New England Biolabs. Chemicals for yeast culture media and assays were from Difco and Sigma. Standard DNA manipulation techniques were applied as described in Sambrook et al. (1989). DNA sequencing was carried out using the Applied Blosystems 373 DNA Sequencer.

Plasmid Constructions

The cDNAs of Medicago cdc2 homologues and cyclins (Magyar et al. 1997) were subcloned from pBluscript II SK plasmids into yeast two-hybrid vectors in the proper frame. The protein encoding region of the cDNAs of Cdc2MsA, Cdc2MsE, MedsaCycB1, MedsaCycA2.2 were cloned into the EcoRI-SaA sites of pGAD424 (Clontech). The protein encoding region the of cDNAs of Cdc2MsB, Cdc2MsD, Cdc2MsF MedsaCycD3 were cloned into the BamHI-SalI sites of pGAD424 (Clontech). Cdc2MsC was cloned into Crf9I-BamHI sites of pGAD424. The Medicago truncatula cyclins were in vector λHybriZAP, a cDNA library was constructed from emerging nodules of Medicago truncatula R108 induced by Sinorhizobium meliloti (Gyorgyey et al. 2000). The protein encoding region the of cDNA CKIMt (WO99/14331) was cloned into the BamHi-SalI sites of pBD-GAL4 (Stratagene)

Yeast Two-Hybrid Analysis

The yeast strain PJ69-4A [MATa trp1-901, leu2-3,112 ura3-52, his3-200 gal4delta, gal80delta GAL2-ADE2, LYS2:: GAL1-HIS3, met2::GAL7-lacZ] (James et al. 1996), which contains three reporter genes (HIS3, ADE2 and lacZ), was used in library screening, growth tests and β-galactosidase assays. Transformation of yeast with pGBT9, pGAD424, pBD-GAL4 and pAD-GAL4 constructs was performed as described by Schiesti and Gietz (1989). The transformation mixture was plated on yeast dropout selection media either lacking leucine and tryptophan (SD-Trp-Leu) for testing the transformation efficiency or on media lacking leucine, tryptophan and histidine, for testing protein-protein interactions. Positive colonies from selective plates were recovered after 4-6 days and their growth tested on selective plates lacking leucine, tryptophan, histidine and adenine. The presence of the plasmids in the transformed yeast cells was confirmed by transforming E.coli with DNA extracted from yeast cells.

To corroborate the interaction between the two fusion proteins in situ, the X-gal (5-bromo4chloro-3-indolyl-β-D-galactopyranoside)-based β-galactosidase assay was used (Bartel et al. 1993). In order to measure the β-galactosidase activity in solution, ONPG (o-nitrophenyl-β-D-galactopyranoside) substrate was used. For liquid phase β-galactosidase measurements yeast cells were treated according to Schneider et al. (1996) with the following modification: yeast cultures were grown in SD-Trp-Leu-His medium to approximately 1 OD600 unit/ml optical density, then two OD600 units of yeast cells were centrifuged (30 sec, 12000 rpm) in 2 ml Eppendorf tubes and lysed.

The lysates were divided into two aliquots, β-galactosidase activity of the first aliquot was measured using ONPG substrate, the second aliquot, that served as blank control in the OD measurement at 420 nm, was treated in the same way, except that the substrate buffer (0.1 M potassium phosphate buffer, pH: 7.0) lacked ONPG. The β-galactosidase activities from three independent measurements were calculated from the OD values as described by Horvath et al. (1998). The standard deviation of the mean values was approximately 15%.

Recombinant CKI Mt

CKI was cloned into BamHI/SalI sites of pET-28a(+) (Novagene) in order to have it in the proper frame. The recombinant plasmid was transformed into E. coli strain BL21 (DE3). The protein expression in the bacteria was induced with 0.5 mM IPTG for 4 hours. Soluble fraction of bacterial rextract was obtained by sonication and affinity purified on Ni-Sepharose. Ni-bound protein was eluted with 300 mM imidazole and dialized overnight at 4° C. against 1×PBS.

Protein Extraction, p13^{suc1}-Sepharose Affinity Binding and Histone H1 Kinase Assays

Alfalfa A2 suspension cells were harvested at indicated time points and either used immediately, or frozen in liquid nitrogen and stored at −80° C. Proteins were extracted by grinding cells with quartz sand in homogenization buffer. Equal amounts of total protein were incubated with 100 μl 25% (v/v) p13^{suc1}-Sepharose beads overnight at 4° C. on a rotary shaker. After incubation, the p13^{suc1}-Sepharose beads were washed and the kinase reaction was initiated by the addition of 30 μl reaction mixture containing a 1 mg/ml histone H1 and 2.5 μCi of [γ-³²P]-ATP. Detailed descriptions of these methods are given in previous publication (Magyar et al. 1993).

Preparation of antibodies, immunoprecipitations and histone H1 and MsRb kinase assays

Polyclonal antibodies against the C-terminal EYFKDIKFVP peptide of alfalfa cdc2Ms A/B (common sequence motif, Hirt et al. 1991, 1993), the C-terminal CFLLENKNQP peptide of B-type alfalfa cyclin cycMs2 (Hirt et al. 1992) as well as against either the C-terminal PYFDSLDKSQY peptide of cdc2MsD or the C-terminal DDLDKTHL peptide of cdc2MsF were raised in rabbits using synthetic peptides coupled to KLH (Keyhole limpet hemocyanin) through an additional Cys residues at the N-terminus. Crude IgG fractions were separated from whole sera by ammonium sulphate fractionation. The IgG fractions were further purified on peptide-coupled affinity columns as described (Harlow and Lane 1988). CycA2.2 policlonal antibody was provided by Dr E. Kondorosi (Roudier et al. 2000)

For immunoprecipitation, equal amounts of protein samples were precleared with 100 μl 25% (v/v) Protein A-Sepharose beads (Pharmacia) for 1 hour at 4° C. on a rotary shaker. After a short centrifugation the supernatants were transfered to new Eppendorf tubes containing 2-4 μg anti-C-terminal alfalfa antibodies (anti-CT cdc2MsA/B or anti-CT cycMs2) and incubated at 4° C. for 1 hr. In the following step, 50 μl 50% (v/v) suspension of Protein A-Sepharose was added and the tubes were gently shaken for 1 hr at 4° C. Thereafter the beads were washed three times and the histone H1 or 22 kDa C-terminal part of MsRb kinase reaction was carried out as described by Magyar et al. (1993).

### Example 10

### Delineation of Immunologically Active Fragments of the MtCycDm Protein

Immunologically active fragments of the MtCycDmn protein of the invention comprise fragments as defined by SEQ ID Nos 31 to 110. Said fragments are linear epitopes consisting of at least seven continuous, contiguous amino acids. Said fragments were defined based on the antigenicity profile calculated using the algorithm described by Parker et al. (1986). Said algorithm is part of the ANTHEPROT software which is made available by e.g. the Pôle Bioinformatique Lyonnais (http://npsa-pbil.ibcp.fr/). Furthermore, the linear epitopes as defined in SEQ ID Nos 31-98 are based on regions of the MtcycDm protein comprising a peak of calculated antigenicity of more than or equal to 50% with the window size parameter set to 7, 15 and 21, respectively. Smaller partially overlapping or adjacent MtCycDm antigenic fragments were furthermore fused to obtain a longer MtCycDm fragment thus comprising at least two of said smaller MtCycDm antigenic fragments.

For single predicted MtCycDm antigenic fragments with a size of larger than 7 contiguous amino acids and smaller than 20 contiguous amino acids, said antigenic fragments were further split up in a set of overlapping core epitopes consisting of 7 contiguous amino acid residues which are possibly extended at the N- and/or C-terminal end with a stretch of 1 or more amino acids outside of the core epitope but part of said antigenic fragment. The example below will clarify this. SEQ ID No 39 for instance is defined as XCELLCGEX (see Table 8 and sequence listing). The ‘X’ residue at position 1 is further defined as either no amino acid or being a stretch of 1 to 2 amino acids, said stretch being (Asp) or (Ser Asp). The ‘X’ residue at position 9 is further defined as either no amino acid or being a stretch of 1 to 2 amino acids, said stretch being (Asp) or (Asp Ser).

Table 8. Overview of predicted MtCycDm antigenic fragments. Given are the amino acid sequences of the antigenic fragments as defined by the corresponding SEQ ID Nos. For amino acid sequences as in SEQ ID Nos 37-110 the amino acids or stretches of amino acids between brackets correspond to the longest extensions (‘X’ at N- and/or C-terminal side) of the given core MtCycDm antigenic fragment (see also text for more detailed explanation). For most MtCycDm antigenic fragments as in SEQ ID Nos 37-110 the extension ‘X’ can also be no amino acid. For a limited set of MtCycDm antigenic fragments as in SEQ ID Nos 37-110, however, the extension ‘X’ should be at least 1 amino acid. If the latter is the case, this is defined in the column ‘Specifics’.

| SEQ ID | | |
|---|---|---|
| NO: | Amino Acid Sequence | Specifics |
| 31 | PAILMPSDCELLCGEDSSEVLTGDLPECSSDLDSS | |
| | SSSQ | |
| 32 | AVFIEHEFKFVPGFDYVSRFQSRSLESSTREEA | |
| 33 | EHEFKFVPGFDYVSRFQ | |
| 34 | LPQLRVTARTTRWSTVSSLSSSPSSSSSPSYSLS- | |
| | YKKRKLNSCFWVDVDKGNSEGREKKQTR | |
| 35 | TARTTRWSTVSSLSSSPSSSSSPSYSLSY | |
| 36 | YSLSYKKRKLNSCFWVDVDKGNSEGREKKQTR | |
| 37 | SDCELLCX (GEDS) | |
| 38 | (S) XDCELLCGX (EDS) | |
| 39 | (SD) XCELLCGEX (DS) | |
| 40 | (SDC) XELLCGEDX (S) | |
| 41 | (SDCE) XLLCGEDS | X at least 1 AA |
| 42/43 | LTGDLPEX (CSSDLDSSSSSQ) | |
| 44/45 | (L) XTGDLPECX (SSDLDSSSSSQ) | |
| 46/47 | (LT) XGDLPECSX (SDLDSSSSSQ) | |
| 48 | (LTG) XDLPECSSX (DLDSSSSSQ) | |
| 49 | (LTGD) XLPECSSDX (LDSSSSSQ) | |
| 50 | (LTGDL) XPECSSDLX (DSSSSSQ) | |
| 51 | (LTGDLP) XECSSDLDX (SSSSSQ) | |
| 52 | (LTGDLPE) XCSSDLDSX (SSSSQ) | |
| 53 | (LTGDLPEC) XSSDLDSSX (SSSQ) | |
| 54/55 | (LTGDLPECS) XSDLDSSSX (SSQ) | |
| 56/57 | (LTGDLPECSS) XDLDSSSSX (SQ) | |
| 58/59 | (LTGDLPECSSD) XLDSSSSSX (Q | |
| 60/61 | (LTGDLPECSSDL) XDSSSSSQ | |
| 62 | LFAEEEEX (DS) | |
| 63 | (L) XFAEEEEDX (S) | |
| 64 | (LF) XAEEEEDS | |
| 65 | RFQSRSLX (ESSTREEA) | X at least 1 AA |
| 66 | (R) XFQSRSLEX (SSTREEA) | |
| 67 | (RF) XQSRSLESX (STREEA) | |
| 68 | (RFQ) XSRSLESSX (TREEA) | |
| 69 | (RFQS) XRSLESSTX (REEA) | |
| 70 | (RFQSR) XSLESSTRX (EEA) | |
| 71 | (RFQSRS) XLESSTREX (EA) | |
| 72 | (RFQSRSL) XESSTREEX (A) | |
| 73 | (RFQSRSLE) XSSTREEA | |
| 74 | VSSNNRRX (NA) | |
| 75 | (V) XSSNNRRNX (A) | |
| 76 | (VS) XSNNRRNA | |
| 77 | RVTARTTX (RW) | |
| 78 | (R) XVTARTTRX (W) | |
| 79 | (RV) XTARTTRW | |
| 80 | SSLSSSPX (SSSSSPS) | |
| 81 | (S) XSLSSSPSX (SSSSPS) | |
| 82 | (SS) XLSSSPSSX (SSSPS) | |
| 83 | (SSL) XSSSPSSSX (SSPS) | X at position |
| | | 1 at least 1 |
| | | AA |
| 84 | (SSLS) XSSPSSSSX (SPS) | X at position |
| | | 9 at least 1 |
| | | AA |
| 85 | (SSLSS) XSPSSSSSX (PS) | |
| 86 | (SSLSSS) XPSSSSSPX (S) | |
| 87 | (SSLSSSP) XSSSSSPS | |
| 88 | YSLSYKKX (RKLN) | |
| 89 | (Y) XSLSYKKRX (KLN) | |
| 90 | (YS) XLSYKKRKX (LN) | |
| 91 | (YSL) XSYKKRKLX (N) | |
| 92 | (YSLS) XYKKRKLN | |
| 93/94 | FWVDVDKX (GNSEGREKKQTR) | |
| 95/96 | (F) XWVDVDKGX (NSEGREKKQTR) | |
| 97 | (FW) XVDVDKGNX (SEGREKKQTR) | |
| 98 | (FWV) XDVDKGNSX (EGREKKQTR) | |
| 99 | (FWVD) XVDKGNSEX (GREKKQTR) | |
| 100 | (FWVDV) XDKGNSEGX (REKKQTR) | |
| 101 | (FWVDVD) XKGNSEGRX (EKKQTR) | |
| 102 | (FWVDVDK) XGNSEGREX (KKQTR) | |
| 103 | (FWVDVDKG) XNSEGREKX (KQTR) | |
| 104 | (FWVDVDKGN) XSEGREKKX (QTR) | |
| 105/106 | (FWVDVDKGNS) XEGREKKQX (TR) | |
| 107/108 | (FWVDVDKGNSE) XGREKKQTX (R) | |
| 109/110 | (FWVDVDKGNSEG) XREKKQTR | |

References

Ach, R. A., Durfee, T., Miller, A. B., Taranto, P., Hanley-Bowdoin, L., Zambryski, P. C., and Gruissem, W. (1997). RRB1 and RRB2 encode maize retinoblastoma-related proteins that interact with a plant D-type cyclin and geminivirus replication protein. Mol. Cell Biol 17, 5077-5086.

Albani, D., Mariconti, L., Ricagno, S., Pitto, L., Moroni, C., and Helin, K. C. R. (2000). DcE2F, a functional plant P9F-like transcriptional activator from Daucus carota. J. Biol Chem. In press.

Alberts, B., Bray, D., Lewis, J., Raff, M., Roberts, K, and Watson, J. D. (1994). “Molecular Biology of the Cell.” Garland Publishing Inc.

Altschul, S. F., Madden, T. L, Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.

An, G., Watson, B. D., Stachel, S., Gordon, M. P., and Nester, E. W. (1985). New cloning vehicles for transformation of higher plants. EMBO J. 4, 277-284.

Armstrong, C. L., Petersen, W. P., Buchholz, W. G., Bowen, B. A., and Sulc, S. L. (1990). Factors affecting PEG-mediated stable transformation of maize protoplasts. Plant Cell Reports 9, 335-339.

Banerjee, A., Pramanik A., Bhattachardya, S., and Balaram, P. (1996). Omega amino acids in peptide design: incorporation into helices. Biopolymers 39, 769-777.

Baron, M. H. and Baltimore, D. (1982). Antibodies against the chemically synthesized genome-linked protein of poliovirus react with native virus-specific proteins. Cell 28, 395-404.

Bartel, P. L., Chien, C.-T., Stemglanz, R., and Fields, S. (1 993). Using the two-hybrid system to detect proteln-protein interactions. In “Cellular interactions in Development: A Practical Approach” (D. A. Hartley, Ed.), pp. 153-179. Oxford University Press, Oxford.

Bartel, P. L. and Fields, S. (1997). “The Yeast Two-Hybrid System.” Oxford University Press.

Bechtold, N. and Pelletier, G. (1998). in planta Agrobacterium-mediated transformation of adult Arabidopsis thaliana plants by vacuum infiltration. Methods Mol. Biol 82, 259-266.

Bell, M. H., Halford, N. G., Ormrod, J. C., and Francis, D. (1993). Tobacco plants transformed with cdc25, a mitotic inducer gene from fission yeast. Plant Mol. Biol 23, 445-451.

Benkirane, N., Guichard, G., Briand, J. P., and Muller, S. (1996). Exploration of requirements for peptidomimetic immune recognition. Antigenic and immunogenic properties of reduced peptide bond pseudopeptide analogues of a histone hexapeptde. J. Biol Chem. 271, 3321 833224.

Berry, A. and Brenner, S. E. (1994). A prototype computer system for de novo protein design. Biochem. Soc. Trans. 22, 10331036.

Binarova, P., Dolezel, J., Draber, P., Heberle-Bors, E., Strnad, M., and Bogre, L. (1998). Treatment of Vicia faba root tip cells with specific inhibitors to cyclin-dependent kinases leads to abnormal spindle formation. Plant J. 16, 697-707.

Bögre, L., Calderini, O., Binarova, P., Mattauch, M., Till, S., Kiegerl, S., Jonak, C., Pollaschek, C., Barker, P., Huskisson, N. S., Hirt, H., and Heberle-Bors, E. (1999). A MAP kinase is activated late in plant mitosis and becomes localized to the plane of cell division. Plant Cell 11, 101-113.

Bögre, L., Zwerger, K., Meskiene, I., Binarova, P., Csizmadla, V., Planck, C., Wagner, E., Hirt, H., and Heberle-Bors, E. (1997). The cdc2Ms kinase is differentially regulated in the cytoplasm and in the nucleus. Plant Physiol. 113, 841-852.

Bradford, M. M. (1976). A rapid and sensitive method for the quantitation of microgram quantities of protein utilizing the principle of protein-dye binding. Anal. Biochem. 72, 248-254.

Calderini, O., Bogre, L., Vicente, O., Binarova, P., Heberle-Bors, E., and Wilson, C. (1998). A cell cycle regulated MAP kinase with a possible role in cytokinesis in tobacco cells. J. Cell Sci. 111,3091-3100.

Christou, P., McCabe, D. E., and Swain, W. F. (1988). Stable transformation of soybean callus by DNA-coated gold particles. Plant Physiol. 87, 671-674.

Church, G. M. and Gilbert, W. (1984). Genomic sequencing. Proc. Natl. Acad. Sci. U.S.A 81,1991-1995.

Cockcroft, C. E., den Boer, B. G. W., Healy, J. M. S., and Murray, J. A. H. (2000). Cyclin D control of growth rate in plants. Nature 405, 575-579.

Cohen-Fix, O. and Koshland, D. (1997). The metaphase-to-anaphase transition: avoiding a mid-life crisis. Curr. Opin. Cell Biol 9, 800-806.

Colasanti, J., Tyers, M., and Sundaresan, V. (1991). Isolation and characterization of cDNA clones encoding a functional p34cdc2 homologue from Zea mays. Proc. Natl. Acad. Sci. U.S.A 88, 3377-3381.

Crossway, A., Oakes, J. V., Irvine, J. M., Ward, B., Knauf, V. C., and Shewmaker, C. K. (1986). integration of foreign DNA following microinjection of tobacco mesophyll protoplasts. Mol. Gen. Genet. 202, 179-185.

Dahl, M., Meskiene, I., Bogre, L., Ha, D. T., Swoboda, I., Hubmann, R., Hirt, H., and Heberle-Bors, E. (1995). The D-type alfalfa cyclin gene cycMs4 complements G1 cyclin-deficient yeast and is induced in the G1 phase of the cell cycle. Plant Cell 7, 1847-1857.

Dale, E. C. and Ow, D. W. (1990). Intra- and intermolecular site-specific recombination in plant cells mediated by bacteriophage P1 recombinase. Gene 91, 79-85.

De Veylder, L., de Almeida, E. J., Burssens, S., Manevstd, A., Lescure, B., Van Montagu, M., Engler, G., and inze, D. (1999). A new D-type cyclin of Arabidopsis thaliana expressed during lateral root primordia formation. Planta 208, 453-462.

Dodds, J. H. (1985). “Plant genetic engineering.” Cambridge University Press.

Doerner, P., Jorgensen, J. E., You, R., Steppuhn, J., and Lamb, C. (1996). Control of root growth and development by cyclin expression. Nature 380, 520-523.

Domer, B., Husar, G. M., Ostresh, J. M., and Houghten, R. A. (1996). The synthesis of peptidomimetic combinatorial libraries through successive amide alkylations. Bioorg. Med. Chem. 4, 709-715.

Elledge, S. J. (1996). Cell cycle checkpoints: preventing an identity crisis. Science 274, 1664-1672.

Ellis, J. G., Llewellyn, D. J., Dennis, E. S., and Peacock, W. J. (1987). Maize Adh-1 promoter sequences control anaerobic regulation: addition of upstream promoter elements from constitutive genes is necessary for expression in tobacco. EMBO J. 6, 11-16.

Evans, T., Rosenthal, E. T., Youngblom, J., Distel, D., and Hunt, T. (1983). Cyclin: a protein specified by maternal mRNA in sea urchin eggs that is destroyed at each cleavage division. Cell 33, 389-396.

Fassina, G. and Melli, M. (1994). Identification of interactive sites of proteins and protein receptors by computer-assisted searches for complementary peptide sequences. Immunomethods. 5,114-120.

Fedoroff, N. V. and Smith, D. L. (1993). A versatile system for detecting transposition in Arabidopsis. Plant J. 3, 273-289.

Feiler, H. S. and Jacobs, T. W. (1990). Cell division in higher plants: a cdc2 gene, its 34-kDa product, and histone H1 kinase activity in pea. Proc. Natl. Acad. Sci. U.S.A 87, 5397-5401.

Ferreira, P. C., Hemerly, A. S., Engler, J. D., Van Montagu, M., Engler, G., and Inze, D. (1994). Developmental expression of the arabidopsis cyclin gene cyc1At. Plant Cell 6, 1763-1774.

Ferreira, P. C., Hemerly, A. S., Villarroel, R., Van Montagu, M., and Inze, D. (1991). The Arabidopsis functional homolog of the p34cdc2 protein kinase. Plant Cell 3, 531-540.

Fesquet, D., Labbe, J. C., Derancourt, J., Capony, J. P., Galas, S., Girard, F., Lorca, T., Shuttleworth, J., Doree, M., and Cavadore, J. C. (1 993). The MO15 gene encodes the catalytic subunit of a protein kinase that activates cdc2 and other cyclin-dependent kinases (CDKs) through phosphorylation of Thr161 and its homologues. EMBO J. 12, 3111-3121.

Fisher, D. K., and Guiltinan M. J. (1995). Rapid, efficient production of homozygous transgenic tobacco plants with Agrobacterium tumefaciens: A seed-to-seed protocol. Plant Mol. Biol. Reporter 13, 278-289.

Fobert, P. R. Gaudin, V., Lunness, P., Coen, E. S., and Doonan, J. H. (1 996). Distinct classes of Cdc2-related genes are differentially expressed during the cell division cycle in plants. Plant Cell 8, 1465-1476.

Francis, D., Dudits, D., and Inzé, D. (1998). “Plant Cell Division.” Portland Press London.

Fromm, M., Taylor, L. P., and Walbot, V. (1985). Expression of genes transferred into monocot and dicot plant cells by electroporation. Proc. Natl. Acad. Sci. U.S.A 82, 5824-5828.

Fuerst, R. A., Soni, R., Murray, J. A., and Lindsey, K. (1996). Modulation of cyclin transcript levels in cultured cells of Arabidopsis thaliana. Plant Physiol 112, 1023-1033.

Gaudin, V., Lunness, P. A., Fobert, P. R., Towers, M., Riou-Khamlichi, C., Murray, J. A., Coen, E., and Doonan, J. H. (2000). The Expression of D-Cyclin Genes Defines Distinct Developmental Zones in Snapdragon Apical Meristems and is Locally Regulated by the Cycloidea Gene. Plant Physiol 122, 1137-1148.

Gietz, R. D. and Schiestl, R. H. (1995). Transforming yeast with DNA methods. Mol. Cell Biol. 5, 255-269.

Gorthy, P. and Pudukottah, M. G. (1999). Production of silicon carbide from rice husks. J. Am. Ceramic Soc. 82.

Grafi, G., Burnett, R. J., Helentjaris, T., Larkins, B. A., DeCaprio, J. A., Sellers, W. R., and Kaelin, W. G., Jr. (1996). A maize cDNA encoding a member of the retinoblastoma protein family: involvement in endoreduplication. Proc. Natl. Acad. Sci. U.S.A 93, 8962-8967.

Gyorgyey, J., Vaubert, D., Jimenez-Zurdo, J. I., Charon, C., Troussard, L, Kondorosi, A., and Kondorosi, E. (2000). Analysis of Medicago truncatula nodule expressed sequence tags. Mol. Plant Microbe Interact. 13, 62-71.

Hanahan, D. (1983). Studies on transformation of Escherichia coli with plasmids. J. Mol. Biol 166, 557-580.

Hansen, G. and Chilton, M. D. (1996). “Agrolistic” transformation of plant cells: integration of T-strands generated in planta. Proc. Natl. Acad. Sci. U.S.A 93, 14978-14983.

Hansen, G., Shillito, R. D., and Chilton, M. D. (1997). T-strand integration in maize protoplasts after codelivery of a T-DNA substrate and virulence genes. Proc. Natl. Acad. Sci. U.S.A 94, 11726-11730.

Hanson, B., Engler, D., Moy, Y., Newman, B., Ralston, E., and Gutterson, N. (1999). A simple method to enrich an Agrobacterium-transformed population for plants containing only T-DNA sequences. Plant J. 19, 727-734.

Harlow, E. and Lane, D. (1988). “Antibodies: A Laboratory Manual.” Cold Spring Harbor Laboratory Press.

Hayles, J. and Nurse, P. (1986). Cell cycle regulation in yeast. J. Cell Sci. Suppl4, 155-170.

Hemerly, A. S., Ferreira, P., de Almelda, E. J., Van Montagu, M., Engler, G., and Inze, D. (1 993). Cdc2a expression in Arabidopsis is linked with competence for cell division. Plant Cell 5, 1711-1723.

Herrera-Estrella, L., De Block, M., Messens, E. H. J. P., Van Montagu, M., and Schell, J. (1983). Chimeric genes as dominant selectable markers in plant cells. EMBO J. 2, 987-995.

Herrera-Estrella, L., Depicker, A., Van Montagu, M., and Schell, J. (1983). Expression of chimaeric genes transferred into plant cells using a Ti-plasmid-derived vector. Nature 303, 209-213.

Hirayama, T., Imajuku, Y., Anal, T., Matsui, M., and Oka, A. (1991). Identification of two cell-cycle-controlling cdc2 gene homologs in Arabidopsis thaliana. Gene 105, 159-165.

Hirt, H., Pay, A., Bogre, L., Meskiene, I., and Heberle-Bors, E. (1993). Cdc2MsB, a cognate cdc2 gene from alfalfa, complements the G1/S but not the G2/M transition of budding yeast cdc28 mutants. Plant J. 4, 61-69.

Hirt, H., Pay, A., Gyorgyey, J., Bako, L., Nemeth, K., Bogre, L., Schweyen, R. J., Heberle-Bors, E., and Dudits, D. (1991). Complementation of a yeast cell cyde mutant by an alfalfa cDNA encoding a protein kinase homologous to p34cdc2. Proc. Natl. Acad. Sci. U.S.A 88, 1636-1640.

Hirt, H., Mink, M., Pfosser, M., Bogre, L, Gyorgyey, J., Jonak, C., Gartner, A., Dudits, D., and Heberle-Bors, E (1992). Alfalfa cyclins: differential expression during the cell cycle and in plant organs. Plant Cell 4, 1531-1538.

Hochstrasser, M. (1998). There's the rub: a novel ubiqutin-like modification linked to cell cycle regulation. Genes Dev. 12, 901-907.

Hoffmann, B., Trinh, T. H., Leung, J., Kondorosi, A., and Kondorosi, E. (1997). A new Medicago truncatula line with superior in vitro regeneration, transformation, and symbiotic properties isolated through cell culture selection. Mol. Plant-Microbe Interact. 10, 307-315.

Hoffman, D. L., Laiter, S., Singh, R. K., Vaisman, I. I., and Tropsha, A. (1995). Rapid protein structure classification using one-dimensional structure profiles on the bioSCAN parallel computer. Comput. Appl. Biosci. 11, 675679.

Hofmann, K., Bucher, P., Falquet, L., and Bairoch, A. (1 999). The PROSITE database, its status in 1999. Nucleic Acids Res. 27, 215-219.

Horvath, G. V., Pettko-Szandtner, A., Nikovics, K., Bilgin, M., Boulton, M., Davies, J. W., Gutierrez, C., and Dudits, D. (1998). Prediction of functional regions of the maize streak virus replication—associated proteins by protein-protein interaction analysis. Plant Mol. Biol. 38, 699-712.

Huntley, R., Healy, S., Freeman, D., Lavender, P., de Jager, S., Greenwood, J., Makker, J., Walker, E., Jackman, M., Xie, Q., Bannister, A. J., Kouzarldes, T., Gutierrez, C., Doonan, J. H., and Murray, J. A. (1998). The maize retinoblastoma protein homologue ZmRb-1 is regulated during leaf development and displays conserved interactions with G1/S regulators and plant cyclin D (CycD) proteins. Plant Mol. Biol. 37, 155-169.

James, P., Halladay, J., and Craig, E. A. (1996). Genomic libraries and a host strain designed for highly efficient two-hybrid selection in yeast. Genetics 144, 1425-1436.

John, P. C., Sek, F. J., and Lee, M. G. (1989). A homolog of the cell cycle control protein p34cdc2 participates in the division cycle of Chlamydomonas, and a similar protein is detectable in higher plants and remote taxa. Plant Cell 1, 1185-1193.

John, P. C. L. (1981). “The cell cycle.” Cambridge University Press.

Krek, W. (1998). Proteolysis and the G1-S transition: the SCF connection. Curr. Opin. Genet. Dev. 8, 36-42.

Krens, F. A., Molendijk, L., Wullems, G. J., and Schilperoort, R. A. (1982). In vitro transformation of plant protoplasts with Ti-plasmid DNA. Nature 296, 72-74.

Kumagai, A. and Dunphy, W. G. (1991). The cdc25 protein controls tyrosine dephosphorylation of the cdc2 protein in a cell4ree system. Cell 64, 903-914.

Labbe, J. C., Capony, J. P., Caput, D., Cavadore, J. C., Derancourt, J., Kaghad, M., Lelias, J. M., Picard, A., and Doree, M. (1989). MPF from starfish oocytes at first meiotic metaphase is a heterodimer containing one molecule of cdc2 and one molecule of cyclin B. EMBO J. 8, 3053-3058.

Laemmll, U. K. (1970). Cleavage of structural proteins during the assembly of the head of bacteriophage T4. Nature 227, 680-685.

Lake, R. S. and Salzman, N. P. (1972). Occurrence and properties of a chromatin-associated F1-histone phosphokinase in mitotic Chinese hamster cells. Biochemistry 11, 4817-4826.

Langan, T. A. (1978). Methods for the assessment of site-specific histone phosphorylation. Methods Cell Biol 19, 127-142.

Lee, M. G. and Nurse, P. (1987). Complementation used to clone a human homologue of the fission yeast cell cycle control gene cdc2. Nature 327, 31-35.

Lerner, R. A. (1982). Tapping the immunological repertoire to produce antibodies of predetermined specificity. Nature 299, 593-596.

Lerner, R. A., Green, N., Alexander, H., Liu, F. T., Sutcliffe, J. G., and Shinnick, T. M. (1981). Chemically synthesized peptides predicted from the nucleotide sequence of the hepatitis B virus genome elicit antibodies reactive with the native envelope protein of Dane particles. Proc. Natl. Acad. Sci. U.S.A 78, 3403-3407.

Liddle, J. E. and Cryer, A. (1991). “A Practical Guide to Monoclonal Antibodies.” Wiley New York.

Lisztwan, J., Marti, A., Sutterduty, H., Gstaiger, M., Wirbelauer, C., and Krek, W. (1998). Association of human CUL-1 and ubiquitin-conjugating enzyme CDC34 with the F-box protein p45(SKP2): evidence for evolutionary conservation in the subunit composition of the CDC34-SCF pathway. EMBO J. 17, 368-383.

Loffler, J., Langui, D., Probst, A., and Huber, G. (1994). Accumulation of a 50 kDa N-terminal fragment of beta-APP695 in Alzheimer's disease hippocampus and neocortex. Neurochem. Int. 24, 281-288.

Lundgren, K., Walworth, N., Booher, R., Dembski, M., Kirschner, M., and Beach, D. (1991). Mik1 and wee1 cooperate in the inhibitory tyrosine phosphorylatlon of cdc2, Cell 64, 1111-1122.

Maes, M. and Messens, E. (1992). Phenol as grinding material in RNA preparations. Nucleic Acids Res. 20, 4374.

Magyar, Z., Bako, L, Bögre, L., Dedeoglu, D., Kapros, T., and Dudits, D. (1993). Active cdc2 genes and cell cycle phase-speciflc cdc2-related kinase complexes in hormone stimulated alfalfa cells. Plant J 4, 151-161.

Magyar, Z., Meszaros, T., Miskolczi, P., Deak, M., Feher, A., Brown, S., Kondorosi, E., Athanasladis, A., Pongor, S., Bilgin, M., Bako, L., Koncz, C., and Dudits, D. (1997). Cell cycle phase specificity of putative cyclin-dependent kinase variants in synchronized alfalfa cells. Plant Cell 9, 223-235.

Merrifield, R. B. (1963). Solid phase peptide synthesis. I. The synthesis of a tetrapeptide. J. Amer. Chem. Soc. 85, 2149-2154.

Mironov, V., De Veylder, L, Van Montagu, M., and Inze, D. (1999). Cyclin-dependent kinases and cell division in plants—the nexus. Plant Cell 11, 509-522.

Monge, A., Lathrop, E. J., Gunn, J. R., Shenkin, P. S., and Friesner, R. A. (1995). Computer modeling of protein folding: conformational and energetic analysis of reduced and detailed protein models. J. Mol. Biol 247, 995-1012.

Muller, H. and Helin, K. (2000). The E2F transcription factors: key regulators of cell proliferation. Biochim. Biophys. Acta 1470, M1-M12.

Murakami, T., Simonds, W. F., and Spiegel, A. M. (1992). Site-specific antibodies directed against G protein beta and gamma subunits: effects on alpha and beta gamma subunit interaction. Biochemistry 31, 2905-2911.

Murray, A. W. and Kirschner, M. W. (1989). Dominoes and clocks: the union of two views of the cell cycle. Science 246, 614-621.

Nakagami, H., Sekine, M., Murakami, H., and Shinmyo, A. (1999). Tobacco refinoblastoma-related protein phosphorylated by a distinct cyclin-dependent kinase complex with Cdc2/cyclin D in vitro. Plant J. 18, 243-252.

Nasmyth, K. (1993). Control of the yeast cell cycle by the Cdc28 protein kinase. Curr. Opin. Cell Biol 5, 166-179.

Needleman, S. B. and Wunsch, C. D. (1970). A general method applicable to the search for similarities in the amino acid sequence of two proteins. J Mol. Biol. 48, 443-453.

Norbury, C. and Nurse, P. (1992). Animal cell cycles and their control. Annu. Rev. Biochem. 61, 441-470.

Nurse, P. (1990). Universal control mechanism regulating onset of M-phase. Nature 344, 503-508.

Nurse, P. and Blsseft, Y. (1981). Gene required in G1 for commitment to cell cycle and in G2 for control of mitosis in fission yeast. Nature 292, 558-560.

Olsen, O. A., Linnestad, C., and Nichols, S. E. (1999). Developmental biology of the cereal endosperm. Trends Plant Sci. 4, 253-257.

Olszewski, K. A., Kolinski, A., and Skolnick, J. (1996). Folding simulations and computer redesign of protein A three-helix bundle motifs. Proteins 25, 286-299.

Ormrod, J. C. and Francis, D. (1993). “Molecular and Cell Biology of the Plant Cell Cycle.” Kluwer Academic Publishers.

Osborne, B. I., Wirtz, U., and Baker, B. (1995). A system for insertional mutagenesis and chromosomal rearrangement using the Ds transposon and Cre-lox. Plant J. 7, 687-701.

Ostresh, J. M., Blondelle, S. E., Dorner, B., and Houghten, R. A. (1996). Generation and use of nonsupport-bound peptide and peptidomimetic combinatorial libraries. Methods Enzymol. 267, 220-234.

Pabo, C. O. and Suchanek, E. G. (1986). Computer-aided model-building strategies for protein design. Biochemistry 25, 5987-5991.

Parker, J. M., Guo, D., and Hodges, R. S. (1986). New hydrophilicity scale derived from high-performance liquid chromatography peptide retention data: correlaton of predicted surface residues with antigenloity and X-ray-derived accessible sites. Biochemistry 25, 5425-5432.

Paszkowski, J., Shillito, R. D., Saul, M., Mandak, V., and Hohn, T. H. B. P. I. (1984). Direct gene transfer to plants. EMBO J. 3, 2717-2722.

Pines, J. (1995). Cyclins and cyclin-dependent kinases: a biochemical view. Biochem. J. 308, 697-711.

Poon, R. Y., Yamashita, K., Adamczewski, J. P., Hunt, T., and Shuttleworth, J. (1993). The cdc2-related protein p40MO15 is the catalytic subunit of a protein kinase that can activate p33cdk2 and p34cdc2. EMBO J. 12, 3123-3132.

Reed, S. I. (1996). G1/S regulatory mechanisms from yeast to man. Prog. Cell Cycle Res. 2, 15-27.

Reed, S. I., Hadwiger, J. A., and Lorincz, A. T. (1985). Protein kinase activity associated with the product of the yeast cell division cycle gene CDC28. Proc. Natl. Acad. Sci. U.S.A 82,4055-4059.

Renaudin, J. P., Doonan, J. H., Freeman, D., Hashimoto, J., Hirt, H., Inze, D., Jacobs, T., Kouchi, H., Rouze, P., Sauter, M., Savoure, A., Sorrell, D. A., Sundaresan, V., and Murray, J. A. (1996). Plant cyclins: a unified nomenclature for plant A-, B- and D-type cyclins based on sequence organization. Plant Mol. Biol. 32, 1003-1018.

Renouf, D. V. and Hounsell, E. F. (1995). Molecular modelling of glycoproteins by homology with non-glycosylated protein domains, computer simulated glycosylation and molecular dynamics. Adv. Exp. Med. Biol 376, 37-45.

Riou-Khamlichi, C., Huntley, R., Jacqmard, A., and Murray, J. A. (1999). Cytokinin activation of Arabidopsis cell division through a D-type cyclin. Science 283, 1541-1544.

Robertson, A. J., Kapros, T., Dudits, D., and Waterborg, J. H. (1996). Identification of three highly expressed replacement histone H3 genes of alfalfa. DNA Seq. 6,137-146.

Rogers, S., Wells, R., and Rechsteiner, M. (1986). Amino acid sequences common to rapidly degraded proteins: the PEST hypothesis. Science 234, 364-368.

Rose, R. B., Craik, C. S., Douglas, N. L., and Stroud, R. M. (1996). Three-dimensional structures of HIV-1 and SIV protease product complexes. Biochemistry 35, 12933-12944.

Russell, P. and Nurse, P. (1986). Cdc25+ functions as an inducer in the mitotic control of fission yeast. Cell 45, 145-153.

Roudler, F., Fedorova, E., Gyorgyey, J., Feher, A., Brown, S., Kondorosi, A., and Kondorosi, E. (2000). Cell cycle function of a Medicago sativa A2-type cyclin interacting with a PSTAIRE-type cyclin-dependent kinase and a retinoblastoma protein. Plant J 23, 73-83.

Russell, P. and Nurse, P. (1987a). Negative regulation of mitosis by wee1+, a gene encoding a protein kinase homolog. Cell 49, 559-567.

Russell, P. and Nurse, P. (1987b). The mitotic inducer nim1+ functions in a regulatory network of protein kinase homologs controlling the initiation of mitosis. Cell 49, 569-576.

Rutenber, E. E., McPhee, F., Kaplan, A. P., Gallion, S. L., Hogan, J. C., Jr., Craik, C. S., and Stroud, R. M. (1996). A new class of HIV-1 protease inhibitor the crystallographic structure, inhibition and chemical synthesis of an aminimide peptide isostere. Bioorg. Med. Chem. 4, 1545-1558.

Sambrook, J., Fritsch, E. F., and Maniatis, T. (1989). “Molecular Cloning: A Laboratory Manual.” Cold Spring Harbor Laboratory Press.

Sanger, F., Nicklen, S., and Coulson, A. R. (1977). DNA sequencing with chain-terminating inhibitors. Proc. Nat. Acad. Sci. U.S.A 74, 5463-5467.

Savouré, A., Kalo, P., Fehér, A., Petrovics, G., Csanadi, G., Szécsi, J., Kiss, G., Brown, S., Kondorosi, A., and Kondorosi, E. (1995). Isolation of a full length mitotic cyclin cDNA clone Cyc III Ms from Medicago sativa: Chromosomal mapping and expression. Plant Mol. Biol. 2, 1059-1070.

Schiestl, R. H. and Gietz, R. D. (1989). High efficiency transformation of intact yeast cells using single stranded nucleic acids as a carrier. Curr. Genet. 16, 339-346.

Schlappi, M., Smith, D., and Fedoroff, N. (1993). TnpA trans-activates methylated maize Suppressor-mutator transposable elements in transgenic tobacco. Genetics 133, 1009-1021.

Schneider, S., Buchert, M., and Hovens, C. M. (1996). An in vitro assay of beta-galactosidase from yeast. Biotechniques 20, 960-962.

Van Montagu, M., and Inze, D. (1996). The Arabidopsis cyclin-dependent kinase gene cdc2bAt is preferentially expressed during S and G2 phases of the cell cycle. Plant J. 10, 601-612.

Sekine, M., Ito, M., Uemukal, K., Maeda, Y., Nakagami, H., and Shinmyo, A. (1999). Isolation and characterization of the E2F-like gene in plants. FEBS Lett. 460, 117-122.

Semler, B. L., Anderson, C. W., Hanecak, R., Dorner, L. F., and Wimmer, E. (1982). A membrane-associated precursor to poliovirus VPg identified by immunoprecipitation with antibodies directed against a synthetic heptapeptide. Cell 28, 405-412.

Shaul, O., Mironov, V., Burssens, S., Van Montagu, M., and Inze, D. (1996). Two Arabidopsis cyclin promoters mediate distinctive transcriptional oscillation in synchronized tobacco BY-2 cells. Proc. Natl. Acad. Sci. U.S.A 93,4868-4872.

Shioda, T., Andriole, S., Yahata, T., and Isselbacher, K. J. (2000). A green fluorescent protein-reporter mammalian two-hybrid system with extrachromosomal maintenance of a prey expression plasmid: Application to interaction screening. Proc. Natl. Acad. Sci. U.S.A 97, 5220-5224.

Soni, R., Carmichael, J. P., Shah, Z. H., and Murray, J. A. (1995). A family of cyclin D homologs from plants differentially controlled by growth regulators and containing the conserved retinoblastoma protein interaction motif. Plant Cell 7, 85-103.

Sorrell, D. A., Combettes, B., Chaubet-Gigot, N., Gigot, C., and Murray, J. A. (1999). Distinct cyclin D genes show mitotic accumulation or constant levels of transcripts in tobacco bright yellow-2 cells. Plant Physiol 119, 343-352.

Sun, Y., Dilkes, B. P., Zhang, C., Dante, R. A., Cameiro, N. P., Lowe, K S., Jung, R., Gordon-Kamm, W. J., and Larkins, B. A. (1999). Characterization of maize (Zea mays L.) Wee1 and its activity in developing endosperm. Proc. Natl. Acad. Sci. U.S.A 96, 4180-4185.

Swenson, K. I., Farrell, K. M., and Ruderman, J. V. (1986). The clam embryo protein cyclin A induces entry into M phase and the resumption of meiosis in Xenopus oocytes. Cell 47, 861-870.

Tamrakar, S., Rubin, E., and Ludlow, J. W. (2000). Role of pRB dephosphorylation in cell cycle regulation. Frontiers Biosci. 5, d121-d137.

Tamura, R. N., Cooper, H. M., Collo, G., and Quaranta, V. (1991). Cell type-specific integrin variants with alternative alpha chain cytoplasmic domains. Proc. Natl. Acad. Sci. U.S.A 88,10183-10187.

Traas, J., Hulskamp, M., Gendreau, E., and Hofte, H. (1998). Endoreduplication and development: rule without dividing? Curr. Opin. Plant Biol 1, 498-503.

Trinh, T. H., Ratet, P., Kondorosi, E., Durand, P., Kamate, P., Bauer, P. and Kondorosi, A. (1998). Rapid and efficient transformation of diploid Medicago truncatula and Medicago sativa ssp. falcata lines improved in somatic embryogenesis. Plant Cell Reports 17,345-355.

Toki, S. (1997). Rapid and efficient Agrobacterium-mediated transformation in rice. Plant Mol. Biol. Reporter 15, 16-21.

Umeda, M., Iwamoto, N., Umeda-Hara, C., Yamaguchi, M., Hashimoto, J., and Uchimiya, H. (1999). Molecular characterization of mitotic cyclins in rice plants. Mol. Gen. Genet. 262, 230-238.

Van Sluys, M. A., Tempe, J., and Fedoroff, N. (1987). Studies on the introduction and mobility of the maize Activator element in Arabidopsis thaliana and Daucus carota. EMBO J. 6, 3881-3889.

Wang, H., Fowke, L C., and Crosby, W. L. (1997). A plant cyclin-dependent kinase inhibitor gene. Nature 386,451-452.

Wilson, A., Pfosser, M., Jonak, C., Hirt, H., Hebere-Bors, E., and Vicente, O. (1999). Evidence for the activation of a MAP kinase upon phosphate-induced cell cycle re-entry in tobacco cells. Physiol. Plant. 102, 532-538.

Wodak, S. J. (1987). Computer-aided design in protein engineering. Ann. N.Y. Acad. Sci. 501, 1-13.

Woulfe, J., Lafortune, L, de Nadai, F., Kitabgi, P., and Beaudet, A. (1994). Post-translational processing of the neurotensin/neuromedin N precursor in the central nervous system of the rat-II. Immunohistochemical localization of maturation products. Neuroscience 60, 167-181.

Xie, Q., Sanz-Burgos, A. P., Hannon, G. J., and Gutierrez, C. (1996). Plant cells contain a novel member of the retinoblastoma family of growth regulatory proteins. EMBO J. 15, 4900-4908.

Xie, Q., Suarez-Lopez, P., and Gutierrez, C. (1995). Identification and analysis of a retinoblastoma binding motif in the replication protein of a plant DNA virus: requirement for efficient viral DNA replication. EMBO J. 14, 4073-4082.

Zeng, Y., Forbes, K. C., Wu, Z, Moreno, S., Piwnica-Worms, H., and Enoch, T. (1998). Replication checkpoint requires phosphorylation of the phosphatase Cdc25 by Cds1 or Chk1. Nature 395, 507-510.

Zhang, Y. L., Dawe, A. L., Jiang, Y., Becker, J. M., and Naider, F. (1996). A superactive peptidomimetic analog of a farnesylated dodecapeptide yeast pheromone. Biochem. Biophys. Res. Commun. 224, 327-331.

1. An isolated nucleic acid encoding a novel plant type D-cyclin or encoding an immunologically active or a functional fragment of such a protein selected from the group consisting of:
(a) nucleic acids comprising at least 41 contiguous nucleotides of the DNA sequence as given in SEQ ID NO 1, or the complement thereof,
(b) nucleic acids comprising the RNA sequences corresponding to at least part of SEQ ID NO 1, or the complement thereof,
(c) nucleic acids specifically hybridising with the nucleotide sequence as defined in any one of SEQ ID NOs 1, or 16 to 21,
(d) nucleic acids encoding a protein with an amino acid sequence which is at least 55% identical to the amino acid sequence as given in SEQ ID NO 2,
(e) nucleic acids encoding a protein comprising the amino acid sequence as given in any of SEQ ID NOs 2 to 6 or 28 to 36
(f) nucleic acids which are degenerated as a result of the genetic code to a nucleic acid encoding a protein as given in SEQ ID NO 2 or as defined in (a) to (e),
(g) nucleic acids which are diverging due to the differences in codon usage between the organisms to a nucleic acid encoding a protein as given in SEQ ID NO 2 or to a nucleic acid as defined in (a) to (e),
(h) nucleic acids which are diverging due to the differences between alleles encoding a protein as given in SEQ ID NO 2 or as defined in (a) to (e),
(i) nucleic acids encoding a fragment of at least 15 amino acids, of a protein as given in any of SEQ ID NOs 2, or 28 to 36,
(j) nucleic acids specifically hybridizing to a nucleic acid encoding a peptide as given in any of SEQ ID NOs 3 to 6 or 28 to 36, and
(k) nucleic acids sequences encoding a protein as defined in SEQ ID NO 2 or as defined in any one of (a) to (j) interrupted by intervening DNA sequences.

2. An isolated nucleic acid according to claim 1 which is DNA, cDNA, genomic DNA or synthetic DNA, or RNA wherein T is replaced by U.

3. A nucleic acid molecule of at least 15 nucleotides in length hybridizing specifically with a nucleic acid of claim 1 or 2.

4. A nucleic acid molecule of at least 15 nucleotides in length specifically amplifying a nucleic acid of claim 1 or 2.

5. A vector comprising a nucleic acid sequence according to claim 1 or 2.

6. A vector according to claim 5 which is an expression vector wherein the nucleic acid sequence is operably linked to one or more control sequences allowing the expression in prokaryotic and/or eukaryotic host cells.

7. A host cell containing a nucleic acid molecule according to claim 1 or 2 or a vector according to claim 5 or 6.

8. The host cell of claim 7, wherein the host cell is a bacterial, insect, fungal, plant or animal cell.

9. An isolated polypeptide encodable by a nucleic acid of claim 1 or 2, or a homologue or a derivative thereof, or an immunologically active or a functional fragment thereof.

10. The polypeptide of claim 9 which has an amino acid sequence as given in SEQ ID No 2, or a homologue or a derivative thereof, or an immunologically active or a functional fragment thereof.

11. The polypeptide of claim 9 which has the ability to form a complex with a cyclin dependent kinase or a cyclin dependent kinase inhibitor.

12. The polypeptide according to claim 11 wherein said cyclin dependent kinase is chosen from the group of plant B-type PPTTLRE and PPTALRE Cdc2 kinases and wherein the cyclin dependent kinase inhibitor is the barrel medic cyclin dependent kinase inhibitor or a functional homologue thereof.

13. A method for producing a polypeptide according to any of claims 9 to 12 comprising culturing a host cell of claim 7 or 8 under conditions allowing the expression of the polypeptide and recovering the produced polypeptide from the culture.

14. An antibody specifically recognizing a plant type D-cyclin of any of claims 9 to 12 or a specific epitope thereof.

15. A method for the production of transgenic plants, plant cells or plant tissues comprising the introduction of a nucleic acid molecule according to any of claims 1 to 2 in an expressible format or a vector according to claim 5 or 6 in said plant, plant cell or plant tissue.

16. A method for effecting the expression of a plant type D cyclin comprising the introduction of a polypeptide of any of claims 9 to 12 directly into a cell, a tissue or an organ of said plant.

17. A method for effecting the expression of a plant type D-cyclin comprising the introduction of a nucleic acid molecule of claim 1 or 2 operably linked to one or more control sequences or a vector according to claim 5 or 6 stably into the genome of a plant cell.

18. The method of claim 17 further comprising regenerating a plant from said plant cell.

19. A transgenic plant cell comprising a nucleic acid sequence of claim 1 or 2 which is operably linked to regulatory elements allowing transcription and/or expression of said nucleic acid in plant cells or obtainable by a method of claim 15 or 18.

20. The transgenic plant cell of claim 19 wherein said nucleic acid of claim 1 or 2 is stably integrated into the genome of said plant cell.

21. A transgenic plant or plant tissue comprising plant cells of claim 19 or 20.

22. A harvestable part of a plant of claim 21.

23. The harvestable part of claim 22 which is selected from the group consisting of seeds, leaves, fruits, stem cultures, rhizomes and bulbs.

24. The progeny derived from any of the plants or plant parts of any of claims 21 to 23.

25. A method for modifying cell fate comprising the modification of expression in particular cells, tissues or organs of a plant, of a nucleic acid sequence of claim 1 or 2.

26. A method for modifying plant development comprising the modification of expression in particular cells, tissues or organs of a plant, of a nucleic acid sequence of claim 1 or 2.

27. A method for modifying plant morphology comprising the modification of expression in particular cells, tissues or organs of a plant, of a nucleic acid sequence of claim 1 or 2.

28. A method for modifying plant biochemistry comprising the modification of expression in particular cells, tissues or organs of a plant, of a nucleic acid sequence of claim 1 or 2.

29. A method for modifying plant physiology comprising the modification of expression in particular cells, tissues or organs of a plant, of a nucleic acid sequence of claim 1 or 2.

30. A method for modifying the cell cycle progression rate comprising the ectopic expression of a plant type D-cyclin of any of claim 9 to 12.

31. A method for obtaining enhanced growth, and/or increased yield and/or delayed senescence of a plant cell, tissue and/or organ and/or increased frequence of formation of lateral organs in a plant, comprising the ectopic expression of a plant type D-cyclin of any of claims 9 to 12.

32. A method for promoting and extending cell division activity in cells in adverse growth conditions and/or in stress, comprising the ectopic expression of a plant type D-cyclin of any of claims 9 to 12.

33. A method for modification of the cell cycle progression rate comprising enhancing the cell cycle phase-specific expression of a plant type D-cyclin of any of claims 9 to 12.

34. A method of any of claims 25 to 33 further comprising the co-expression of a target protein of said D-cyclin.

35. The method of claim 34 wherein said target protein is a Cdc2-type or Cdc2-related kinase chosen from the group consisting of B-type PPTTLRE Cdc2, B-type PPTALRE Cdc2 and A-type PSTAIRE Cdc2.

36. The method of claim 34 or 35 wherein the expression of said target proteins is enhanced during the phases of natural expression of said target protein, possibly by gene duplication as a result of the introduction of a recombinant gene copy of said target protein.

37. The method of any of claims 25 to 33 further comprising the downregulation of the expression of a target protein of said D-cyclin.

38. The method of claim 37 wherein said target protein is the barrel medic cyclin dependent kinase inhibitor or a functional homologue thereof.

39. A method for modification of the cell cycle progression rate comprising downregulation of the expression of a plant type D-cyclin of any of claims 9 to 12.

40. The method of claim 39 wherein said downregulation involves the downregulation of the cell cycle specific expression of said D-cyclin.

41. The method of claim 39 or 40 wherein the expression of said D-cyclin is downregulated during the M-phase of the cell cycle.

42. A method for creating dwarfism comprising the downregulation of the expression of a plant D-cyclin of any of claims 9 to 12.

43. A method for creating inhibition of side shoot formation in plant, plant tissues or organs comprising the downregulation of the expression of a plant D-cyclin of any of claims 9 to 12.

44. A method for conferring enhanced resistance to pathogens causing neoplastic growth comprising the downregulation of the expression of a plant D-cyclin of any of claims 9 to 12.

45. A method for increasing seed yield and/or seed size comprising the downregulation of the expression of a plant D-cyclin of any of claims 9 to 12.

46. A method for enhancing endoreduplication comprising the downregulation of the expression of a plant D-cyclin of any of claims 9 to 12.

47. A method of any of claims 39 to 46 further comprising the ectopic expression of a target protein of said D-cyclin.

48. The method of claim 47 wherein said target is the barrel medic cyclin dependent kinase inhibitor or a functional homologue thereof.

49. A method for identifying and obtaining proteins interacting with plant type D-cyclin comprising a two-hybrid screening assay wherein a polypeptide of any of claims 9 to 12 as a bait and a cDNA library as prey are used.

50. A method for modulating the interaction between and/or for modulating the activity of complexes comprising type D-cyclin and Cdc2-type or Cdc2-related kinases and/or other D-cyclin-interacting protein partners obtainable by a method according to claim 49 comprising the use of a polypeptide of any of claims 9 to 12.

51. The method of claim 50 wherein said other D-cyclin-interacting protein partner is the barrel medic cyclin dependent kinase inhibitor or a functional homologue thereof.

52. A method for modulating the interaction between and/or the activity of complexes comprising type D-cyclin and Cdc2-type or Cdc2-related kinases comprising the use of the barrel medic cyclin dependent kinase inhibitor or a functional homologue thereof.

53. A method for identifying and obtaining compounds interacting with plant type D-cyclin comprising the steps of:
a) providing a yeast two-hybrid system wherein a polypeptide of any of claims 9 to 12 and a Cdc2-type or Cdc2-related kinase or D-cyclin-interacting protein partners obtainable by a method according to claim 49 are expressed,
b) interacting said compound with the complex formed by the expressed polypeptides as defined in a).

54. The method of claim 53 wherein said D-cyclin-interacting protein partner is the barrel medic cyclin dependent kinase inhibitor or a functional homologue thereof.

55. A method for identifying compounds or mixtures of compounds which specifically bind to a polypeptide of any of claims 9 to 12, comprising:
a) combining a polypeptide of any of claims 9 to 12 with said compound or mixtures of compounds under conditions suitable to allow complex formation, and,
b) detecting complex formation, wherein the presence of a complex identifies a molecule which specifically binds said polypeptide.

56. Use of a molecule identified by means of a method of any of claims 53 to 55 as a plant growth regulator or herbicide.

57. A method for production of a plant growth regulator or herbicide composition comprising the steps of the method of any of claims 49 or 53 to 55 and formulating the compounds obtained from said steps in a suitable form for the application in agriculture or plant cell or tissue culture.

58. Use of a nucleic acid molecule of any of claims 1 to 3, the vector of claim 5 or 6, a polypeptide of claim 9 to 12 or an antibody of claim 14 for modifying cell fate.

59. Use of a nucleic acid molecule of any of claims 1 to 3, the recombinant vector of claim 5 or 6, a polypeptide of any of claims 9 to 12 or an antibody of claim 14 for modifying plant development.

60. Use of a nucleic acid molecule of any of claims 1 to 3, the recombinant vector of claim 5 or 6, a polypeptide of claim 9 to 12 or an antibody of claim 14 for modifying plant morphology.

61. Use of a nucleic acid molecule of any of claims 1 to 3, the recombinant vector of claim 5 or 6, a polypeptide of claim 9 to 12 or an antibody of claim 14 for modifying plant biochemistry.

62. Use of a nucleic acid molecule of any of claims 1 to 3, the recombinant vector of claim 5 or 6, a polypeptide of claim 9 to 12 or an antibody of claim 14 for modifying plant physiology.

63. Diagnostic composition comprising at least a nucleic acid molecule of any of claims 1 to 4, the vector of claim 5 or 6, a polypeptide of claim 9 to 12 or an antibody of claim 14.
